(19) United States
(12) Patent Application Publication
(10) Pub. No.: US 2024/0182449 A1
(43) Pub. Date: Jun. 6, 2024

(54) CRYSTALLINE FORMS OF AN MK2 INHIBITOR

(71) Applicant: XinThera, Inc., Foster City, CA (US)

(72) Inventors: Jing LIU, San Diego, CA (US); Jayachandra P. REDDY, West Palm Beach, FL (US); Daniel John GANLEY, Syracuse, NY (US); Robert L. HOFFMAN, San Diego, CA (US)

(21) Appl. No.: 18/481,162

(22) Filed: Oct. 4, 2023

Related U.S. Application Data
(60) Provisional application No. 63/413,422, filed on Oct. 5, 2022.

(51) Int. Cl. C07D 401/14

(52) U.S. Cl. CPC C07D 401/14; C07B 2200/13

(57) ABSTRACT

Described herein are crystalline forms of (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1), or a pharmaceutically acceptable salt or solvate thereof.

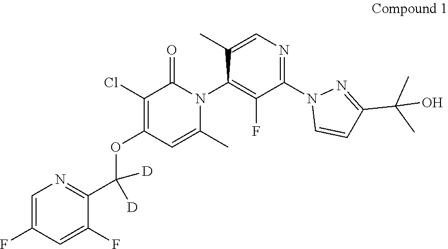

### CROSS-REFERENCE TO RELATED APPLICATIONS

This application claims the benefit under 35 U.S.C. § 119(e) to U.S. Provisional Application No. 63/413,422, filed Oct. 5, 2022, which is hereby incorporated by reference in its entirety.

### BACKGROUND

Mitogen-activated protein kinases (MAPK) are a conserved family of enzymes that relay and propagate external stimuli, using phosphorylation cascades to generate a coordinated cellular response to the environment. The MAPK are proline-directed serine/threonine-specific protein kinases that regulate cellular activities, such as gene expression, mitosis, differentiation, and cell survival/apoptosis. To date, four distinct classes of mammalian MAPK have been identified: the extracellular signaling kinases (ERK1 and 2), the c-jun N-terminal kinase-1 (JNK1-3), the p38 MAPK (p38α, β, γ, and δ), and ERK5. The MAPK are activated by the dual phosphorylation of Thr and Tyr residues within a TXY activation motif by coordinated dual-specificity MAPKK, where X is Glu, Pro, and Gly in ERK, JNK, and p38 MAPK, respectively. MAPK are 60-70% identical to each other yet differ in their activation loop sequences and sizes. The activation loop is adjacent to the enzyme-active site, and its phosphorylation allows the enzyme to reposition active-site residues into the optimal orientation for substrate binding and catalysis. Downstream substrates of MAPK include mitogen-activated protein-kinase-activated protein (MAPKAP) kinases and transcription factors, the phosphorylation of which, either directly or indirectly, regulates gene expression at several points, including transcription, nuclear export, and mRNA stability and translation. The cellular consequences of MAPK activation include inflammation, apoptosis, differentiation, and proliferation.

Distinct genes encode four p38 MAPK in humans: p38α, β, γ, and δ. Significant amino acid sequence homology is observed among the 4 isoforms, with 60-75 overall sequence identity and>90% identity within the kinase domains. Tissue-selective expression is observed, with p38γ found predominantly in skeletal muscle, p38δ in the testes, pancreas, and small intestine. In contrast, p38a and β are more ubiquitously expressed.

p38 MAPK is the major isoform involved in the immune and inflammatory response. As such its function is critical for the production and activity of multiple proinflammatory cytokines, including TNFa, IL-1, IL-6, and IL-8, in cells such as macrophages, monocytes, synovial cells, and endothelial cells. p38 MAPK is also responsible for the induction of key inflammatory enzymes such as COX2 and iNOS, the major sources of eicosanoids and nitric oxide at sites of inflammation, respectively. Additionally, the p38 MAPK pathway regulates the expression of matrix metalloproteinases (MMP), including MMP2, MMP9, and MMP13.

The use of selective and potent inhibitors has facilitated the discovery of several families of p38 MAPK substrates, including transcription factors, MAPKAP kinases, and other enzymes. p38 MAPK can directly phosphorylate several transcription factors, such as myocyte-specific enhancer binding factor 2C (MEF2C), CHOP, peroxisome proliferator-activated receptor (PPAR) a, PPAR γ co-activator 1 and p53. These transcription factors are involved in cellular functions such as apoptosis, gluconeogenesis, and synthesis of enzymes involved in fatty acid oxidation. p38 MAPK is also involved in the direct or indirect phosphorylation of enzyme substrates, such as cytosolic phospholipase A2, and the Cdc25 phosphatases, which are involved in the activation of cyclin-dependent protein kinase activity and cell-cycle regulation. Therefore in addition to its role in the inflammatory response, p38 MAPK has other functions associated with normal and abnormal cell growth and survival as well as cellular function and homeostasis. The MAPKAP kinases (MK2, MK-3, and PRAK) are selectively phosphorylated by p38 MAPK, while the phosphorylation of MSK1/2, MNK1/2, and RSKb is catalyzed by both p38 MAPK and ERK.

MK-2, MK-3, and PRAK, once phosphorylated and activated by p38 MAPK, share similar substrate specificities. All of these kinases can phosphorylate the small heat-shock protein Hsp27. Studies have shown that the PRAK- and MK3-deficient mice do not display any resistance to endotoxic shock or a decrease in lipopolysaccharide-(LPS)-induced cytokine production. In contrast, MK-2-deficient mice show a resistance to endotoxic shock and an impaired inflammatory response, as well as a significantly decreased production of cytokines such as TNFa, IFNy and IL-6. Thus, the p38/MK2 axis is important for mediating pro-inflammatory responses.

The p38:MK2 complex is very stable with a Kd of 6 nM. The binding affinity of p38 for MK2 is driven by the C-terminal domain of MK2 containing several positively charged amino acid residues. Crystallographic studies of the p38:MK2 complex demonstrated that the C- terminal region of MK2 wraps around p38a and binds to the negatively charged ED binding site. The tight binding of p38 to MK2 may give rise to conformational changes providing additional binding pockets for inhibitors that would specifically be dependent upon the p38:MK2 interaction. Taken together, these two studies suggests that selective p38/MK2 axis blockade is achievable with small molecule inhibitors. In comparison to traditional p38 MAPK inhibitors these p38/MK2 inhibitors should retain or enhance potency and exhibit improved safety features in animal models of disease or in human clinical settings.

The p38/MK2 role in the regulation of inflammatory cytokines (TNFa, IL-Iβ, IL-6) and enzymes responsible for inflammation (COX-2, iNOS, and MMPs) makes it an attractive drug target. Several classical p38 MAPK inhibitors have progressed to testing in clinical trials. Some of these candidates have failed, for safety or other reasons, but several have reported clinical data in diseases such as rheumatoid arthritis, pain, Crohn's disease, acute coronary syndrome, multiple myeloma, and chronic obstructive pulmonary disease. In addition to these diseases several IL-Iβ mediated diseases could be impacted by a p38 inhibitor based upon the key role for the p38 MAPK pathway in the biosynthesis and activity of this cytokine. These diseases include the family of cryopyrin associated periodic disorders (CAPS), chronic gout, diabetes, Still's disease, and Familial Mediterranean Fever among others.

Accordingly, there is a need for small molecule inhibitors of MK2 which are useful in treating diseases and conditions associated with the activity of MK2.

### SUMMARY

Disclosed herein is a crystalline form of (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1):

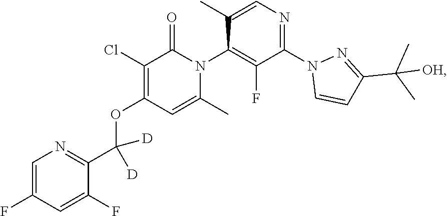

or a pharmaceutically acceptable salt or solvate thereof.

Disclosed herein is a crystalline form of freebase (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1):

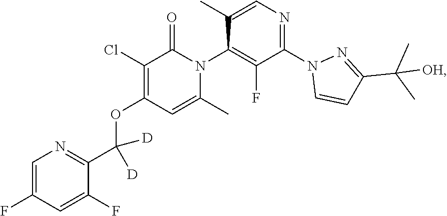

or a pharmaceutically acceptable solvate thereof.

Disclosed herein is a crystalline form of freebase (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1):

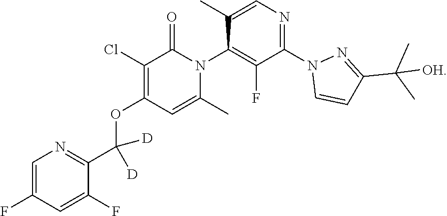

In some embodiments of a crystalline form, the crystalline form is selected from the group consisting of Form I of compound 1, Form II of compound 1, Form III of compound 1, Form IV of compound 1, Form VI of compound 1, Form VII of compound 1, Form VIII of compound 1, Form IX of compound 1, and Form X of compound 1, or any combinations thereof.

In some embodiments of a crystalline form, the crystalline form is selected from the group consisting of freebase Form I of compound 1, freebase Form II of compound 1, freebase Form III of compound 1, freebase Form I of compound 1, freebase Form VI of compound 1, freebase Form VII of compound 1, freebase Form VIII of compound 1, freebase Form IX of compound 1, and freebase Form X of compound 1, or any combinations thereof.

In some embodiments of a crystalline form, the crystalline form is selected from the group consisting of Form I of compound 1, Form IV of compound 1, Form VIII of compound 1, Form IX of compound 1, and Form X of compound I, or any combinations thereof.

In some embodiments of a crystalline form, the crystalline form is selected from the group consisting of freebase Form I of compound 1, freebase Form IV of compound 1, freebase Form VIII of compound 1, freebase Form IX of compound 1, and freebase Form X of compound 1, or any combinations thereof.

In some embodiments of a crystalline form, the crystalline compound 1 is Form X characterized as having at least one of the following properties:
(a) an X-ray powder diffraction (XRPD) pattern substantially the same as shown in FIG. 6;
(b) an X-ray powder diffraction (XRPD) pattern with characteristic peaks at 9.17±0.2° 2θ, 16.29±0.2° 2θ, 21.67±0.2° 2θ, and 23.72±0.2° 2θ.
(c) a DSC thermogram with an endotherm having an onset temperature at about 157° C. and a peak temperature at about 158° C.; or
(d) combinations thereof.

In some embodiments of a crystalline form, the crystalline form has an X-ray powder diffraction (XRPD) pattern substantially the same as shown in FIG. 6.

In some embodiments of a crystalline form, the crystalline form has an X-ray powder diffraction (XRPD) pattern with characteristic peaks at 9.17±0.2° 2θ, 16.29±0.2° 2θ, 21.67±0.2° 2θ, and 23.72±0.2° 2θ.

In some embodiments of a crystalline form, the X-ray powder diffraction pattern further comprises peaks at 13.44±0.2° 2θ, 14.95±0.2° 2θ, and 25.72±0.2° 2θ.

In some embodiments of a crystalline form, the crystalline form has an X-ray powder diffraction (XRPD) pattern with characteristic peaks at 9.17±0.2° 2θ, 13.44±0.2° 2θ, 14.95±0.2° 2θ, 16.29±0.2° 2θ, 21.67±0.2° 2θ, 23.72±0.2° 2θ, and 25.72±0.2° 2θ.

In some embodiments of a crystalline form, the X-ray powder diffraction pattern further comprises peaks at 7.46±0.2° 2θ, 18.14±0.2° 2θ, 20.95±0.2° 2θ, 22.53±0.2° 2θ, and 24.80±0.2° 2θ.

In some embodiments of a crystalline form, the crystalline form has an X-ray powder diffraction (XRPD) pattern with characteristic peaks at 7.46±0.2° 2θ, 9.17±0.2° 2θ, 13.44±0.2° 2θ, 14.95±0.2° 2θ, 16.29±0.2° 2θ, 18.14±0.2° 2θ, 20.95±0.2° 2θ, 21.67±0.2° 2θ, 22.53±0.2° 2θ, 23.72±0.2° 2θ, 24.80±0.2° 2, and 25.72±0.2° 2θ.

In some embodiments of a crystalline form, the X-ray powder diffraction pattern further comprises at least one peak selected from 14.44±0.2° 2θ, 17.15±0.2° 2θ, and 18.74±0.2° 2θ.

In some embodiments of a crystalline form, the crystalline form has a DSC thermogram with an endotherm having an onset temperature at about 157° C. and a peak temperature at about 158° C.

In some embodiments of a crystalline form, the crystalline form is anhydrous.

In some embodiments of a crystalline form, the crystalline form is thermodynamically stable.

In some embodiments of a crystalline form, the crystalline form is non-hygroscopic.

In some embodiments of a crystalline form, the crystalline form is physically and chemically stable.

In some embodiments of a crystalline form, the crystalline compound 1 is Form I characterized as having at least one of the following properties:
(a) an X-ray powder diffraction (XRPD) pattern substantially the same as shown in FIG. 2;
(b) an X-ray powder diffraction (XRPD) pattern with characteristic peaks at 12.02±0.2° 2θ, 13.13±0.2° 2θ, 16.56±0.2° 2θ, 19.20±0.2° 2θ, 26.00±0.2° 2θ, and 28.00±0.2° 2θ;
(c) a DSC thermogram with an endotherm having an onset temperature at about 27° C. and a peak temperature at about 62° C.;
(d) a DSC thermogram with an endotherm having an onset temperature at about 98° C. and a peak temperature at about 104° C.;
(e) a thermogravimetric analysis (TGA) thermogram comprising a loss in mass of about 2.6% over a temperature range of about 25° C. to about 90° C.; or
(f) combinations thereof.

In some embodiments of a crystalline form, crystalline compound 1, Form I has an X-ray powder diffraction (XRPD) pattern substantially the same as shown in FIG. 2.

In some embodiments of a crystalline form, crystalline compound 1, Form I has an X-ray powder diffraction (XRPD) pattern with characteristic peaks at 12.02±0.2° 2θ, 13.13±0.2° 2θ, 15.37±0.2° 2θ; 16.56±0.2° 2θ, 19.20±0.2° 2θ, 26.00±0.2° 2θ, and 28.00±0.2° 2θ.

In some embodiments of a crystalline form, the X-ray powder diffraction pattern further comprises at least one peak selected from 11.30±0.2° 2θ, 13.89±0.2° 2θ, 20.45±0.2° 2θ, and 26.39±0.2° 2θ.

In some embodiments of a crystalline form, crystalline compound 1, Form I has an X-ray powder diffraction (XRPD) pattern with characteristic peaks at 11.30±0.2° 2θ, 12.02±0.2° 2θ, 13.13±0.2° 2θ, 13.89±0.2° 2θ, 15.37±0.2° 2θ; 16.56±0.2° 2θ, 19.20±0.2° 2θ, 20.45±0.2° 2θ, 26.00±0.2° 2θ, 26.39±0.2° 2θ, and 28.00±0.2° 2θ.

In some embodiments of a crystalline form, the X-ray powder diffraction pattern further comprises at least one peak selected from 9.21±0.2° 2θ, 9.57±0.2° 2θ, and 21.81±0.2° 2θ.

In some embodiments of a crystalline form, crystalline compound 1, Form I has a DSC thermogram with an endotherm having an onset temperature at about 27° C. and a peak temperature at about 62° C.

In some embodiments of a crystalline form, crystalline compound 1, Form I has a DSC thermogram with an endotherm having an onset temperature at about 98° C. and a peak temperature at about 104° C.

In some embodiments of a crystalline form, crystalline compound 1, Form I has a DSC thermogram with an endotherm having an onset temperature at about 27° C. and a peak temperature at about 62° C. and an onset temperature at about 98° C. and a peak temperature at about 104° C.

The crystalline form of any one of claim 1-7 or 21-29, wherein crystalline compound 1, Form I has a thermogravimetric analysis (TGA) thermogram comprising a loss in mass of about 2.6% over a temperature range of about 25° C. to about 90° C.

In some embodiments of a crystalline forma crystalline compound 1, Form I is a hydrate.

In some embodiments of a crystalline form, the crystalline compound 1 is Form IV characterized as having at least one of the following properties:
(a) an X-ray powder diffraction (XRPD) pattern substantially the same as shown in FIG. 3;
(b) an X-ray powder diffraction (XRPD) pattern with characteristic peaks at 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.
(c) a DSC thermogram with an endotherm having an onset temperature at about 36° C. and a peak temperature at about 52° C.;
(d) a DSC thermogram with an endotherm having an onset temperature at about 103.5° C. and a peak temperature at about 109° C.;
(e) a thermogravimetric analysis (TGA) thermogram comprising a loss in mass of about 1.5% over a temperature range of about 33° C. to about 100° C.; or
(f) combinations thereof.

In some embodiments of a crystalline forma crystalline compound 1, Form IV has an X-ray powder diffraction (XRPD) pattern substantially the same as shown in FIG. 3.

In some embodiments of a crystalline forma crystalline compound 1, Form IV has an X-ray powder diffraction (XRPD) pattern with characteristic peaks at 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.

In some embodiments of a crystalline form, the X-ray powder diffraction pattern further comprises at least one peak selected from 15.35±0.2° 2θ, 19.52±0.2° 2θ, 19.78±0.2° 2θ, 23.59±0.2° 2θ, 23.86±0.2° 2θ, and 27.88±0.2° 2θ.

In some embodiments of a crystalline form, crystalline compound 1, Form IV has an X-ray powder diffraction (XRPD) pattern with characteristic peaks at 11.27±0.2° 2θ, 12.24±0.2° 2θ, 14.12±0.2° 2θ, 15.35±0.2° 2θ, 19.03±0.2° 2θ, 19.52±0.2° 2θ, 19.78±0.2° 2θ, 20.09±0.2° 2θ, 20.77±0.2° 2θ, 21.33±0.2° 2θ, 23.59±0.2° 2θ, 23.86±0.2° 2θ, 26.37±0.2° 2θ, and 27.88±0.2° 2θ.

In some embodiments of a crystalline form, the X-ray powder diffraction pattern further comprises at least one peak selected from 15.51±0.2° 2θ, 15.79±0.2° 2θ, and 27.32±0.2° 2θ.

In some embodiments of a crystalline form, crystalline compound 1, Form IV has a DSC thermogram with an endotherm having an onset temperature at about 36° C. and a peak temperature at about 52° C.

In some embodiments of a crystalline form, crystalline compound 1, Form IV has a DSC thermogram with an endotherm having an onset temperature at about 103.5° C. and a peak temperature at about 109° C.

In some embodiments of a crystalline form, crystalline compound 1, Form IV has a DSC thermogram with an endotherm having an onset temperature at about 36° C. and a peak temperature at about 52° C. and an onset temperature at about 103.5° C. and a peak temperature at about 109° C.

In some embodiments of a crystalline form, crystalline compound 1, Form IV has a thermogravimetric analysis (TGA) thermogram comprising a loss in mass of about 1.5% over a temperature range of about 33° C. to about 100° C.

In some embodiments of a crystalline form, crystalline compound 1, Form IV is a hydrate.

In some embodiments of a crystalline form, the crystalline compound 1 is Form IV characterized as having at least one of the following properties:
(a) an X-ray powder diffraction (XRPD) pattern substantially the same as shown in FIG. 4;
(b) an X-ray powder diffraction (XRPD) pattern with characteristic peaks at 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.
(c) a DSC thermogram with an endotherm having an onset temperature at about 48° C. and a peak temperature at about 49° C.;
(d) a DSC thermogram with an endotherm having an onset temperature at about 154° C. and a peak temperature at about 155° C.;
(e) a thermogravimetric analysis (TGA) thermogram comprising a loss in mass of about 0.23% over a temperature range of about 30° C. to about 50° C.; or
(f) combinations thereof.

In some embodiments of a crystalline form, crystalline compound 1, Form VIII has an X-ray powder diffraction (XRPD) pattern substantially the same as shown in FIG. 4.

In some embodiments of a crystalline form, compound 1, Form VIII has an X-ray powder diffraction (XRPD) pattern with characteristic peaks at 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.

In some embodiments of a crystalline form, the X-ray powder diffraction pattern further comprises at least one peak selected from 10.43±0.2° 2θ, 12.94±0.2° 2θ, 17.99±0.2° 2θ, 19.57±0.2° 2θ, 21.80±0.2° 2θ, and 27.16±0.2° 2θ.

In some embodiments of a crystalline form, crystalline compound 1, Form VIII has an X-ray powder diffraction (XRPD) pattern with characteristic peaks at 9.23±0.2° 2θ, 10.43±0.2° 2θ, 12.29±0.2° 2θ, 12.94±0.2° 2θ, 13.18±0.2° 2θ, 14.83±0.2° 2θ, 16.01±0.2° 2θ, 16.76±0.2° 2θ, 17.99±0.2° 2θ, 18.48±0.2° 2θ, 19.57±0.2° 2θ, 21.80±0.2° 2θ, 26.84±0.2° 2θ, and 27.16±0.2° 2θ.

In some embodiments of a crystalline form, the X-ray powder diffraction pattern further comprises at least one peak selected from 13.74±0.2° 2θ, 19.14±0.2° 2θ, and 19.96±0.2° 2θ.

In some embodiments of a crystalline form, crystalline compound 1, Form VIII has a DSC thermogram with an endotherm having an onset temperature at about 48° C. and a peak temperature at about 49° C.

In some embodiments of a crystalline form, crystalline compound 1, Form VIII has a DSC thermogram with an endotherm having an onset temperature at about 154° C. and a peak temperature at about 155° C.

In some embodiments of a crystalline form, crystalline compound 1, Form VIII has a DSC thermogram with an endotherm having an onset temperature at about 48° C. and a peak temperature at about 49° C. and an onset temperature at about 154° C. and a peak temperature at about 155° C.

In some embodiments of a crystalline form, crystalline compound 1, Form VIII has a thermogravimetric analysis (TGA) thermogram comprising a loss in mass of about 0.23% over a temperature range of about 30° C. to about 50° C.

In some embodiments of a crystalline form, crystalline compound 1, Form VIII is anhydrous.

In some embodiments of a crystalline form, the crystalline compound 1 is Form IX characterized as having at least one of the following properties:
(a) an X-ray powder diffraction (XRPD) pattern substantially the same as shown in FIG. 5;
(b) an X-ray powder diffraction (XRPD) pattern with characteristic peaks at 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.
(c) a DSC thermogram with an endotherm having an onset temperature at about 29° C. and a peak temperature at about 55.5° C.;
(d) a DSC thermogram with an endotherm having an onset temperature at about 113.5° C. and a peak temperature at about 118° C.;
(e) a thermogravimetric analysis (TGA) thermogram comprising a loss in mass of about 1.39% over a temperature range of about 27° C. to about 80° C.; or
(f) combinations thereof.

In some embodiments of a crystalline form, crystalline compound 1, Form IX has an X-ray powder diffraction (XRPD) pattern substantially the same as shown in FIG. 5.

In some embodiments of a crystalline form, crystalline compound 1, Form IX has an X-ray powder diffraction (XRPD) pattern with characteristic peaks at 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.

In some embodiments of a crystalline form, the X-ray powder diffraction pattern further comprises at least one peak selected from 17.65±0.2° 2θ, 19.19±0.2° 2θ, 20.14±0.2° 2θ, 23.54±0.2° 2θ, 26.65±0.2° 2θ, and 30.1±0.2° 2θ.

In some embodiments of a crystalline form, crystalline compound 1, Form IX has an X-ray powder diffraction (XRPD) pattern with characteristic peaks at 7.59±0.2° 2θ, 13.18±0.2° 2θ, 13.95±0.2° 2θ, 15.48±0.2° 2θ, 17.65±0.2° 2θ, 18.78±0.2° 2θ, 19.19±0.2° 2θ, 20.14±0.2° 2θ, 20.87±0.2° 2θ, 21.62±0.2° 2θ, 23.37±0.2° 2θ, 23.54±0.2° 2θ, 26.65±0.2° 2θ, 28.07±0.2° 2θ, and 30.1±0.2° 2θ.

In some embodiments of a crystalline form, the X-ray powder diffraction pattern further comprises at least one peak selected from 11.09±0.2° 2θ, 27.49±0.2° 2θ, and 30.99±0.2° 2θ.

In some embodiments of a crystalline form, crystalline compound 1, Form IX has a DSC thermogram with an endotherm having an onset temperature at about 29° C. and a peak temperature at about 55.5° C.

In some embodiments of a crystalline form, crystalline compound 1, Form IX has a DSC thermogram with an endotherm having an onset temperature at about 113.5° C. and a peak temperature at about 118° C.

In some embodiments of a crystalline form, crystalline compound 1, Form IX has a DSC thermogram with an endotherm having an onset temperature at about 29° C. and a peak temperature at about 55.5° C. and an onset temperature at about 113.5° C. and a peak temperature at about 118° C.

In some embodiments of a crystalline form, crystalline compound 1, Form IX has a thermogravimetric analysis (TGA) thermogram comprising a loss in mass of about 1.39% over a temperature range of about 27° C. to about 80° C.

In some embodiments of a crystalline form, crystalline compound 1, Form IX is a hydrate.

Also disclosed herein is a pharmaceutical composition comprising a therapeutically effective amount of a crystalline form disclosed herein, and a pharmaceutically acceptable excipient.

Also disclosed herein is a method for treating a condition comprising administering to a subject in need thereof a therapeutically effective amount of a crystalline form disclosed herein, wherein the condition is selected from the group consisting of an autoimmune disorder, a chronic inflammatory disorder, an acute inflammatory disorder, an auto-inflammatory disorder, a fibrotic disorder, a metabolic disorder, a neoplastic disorder, and a cardiovascular or a cerebrovascular disorder.

Also disclosed herein is a method of treating a p38 MAP kinase-mediated disease in a subject in need thereof comprising administering to the subject a therapeutically effective amount of a crystalline form disclosed herein.

Also disclosed herein is method of treating a MK2-mediated disease in a subject in need thereof comprising administering to the subject a therapeutically effective amount of a crystalline form disclosed herein.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1. shows the compound 1 single crystal structure.

FIG. 2 shows the X-ray powder diffraction (XRPD) pattern for crystalline compound 1, Form I.

FIG. 3 shows the X-ray powder diffraction (XRPD) pattern for crystalline compound 1, Form IV.

FIG. 4 shows the X-ray powder diffraction (XRPD) pattern for crystalline compound 1, Form VIII.

FIG. 5 shows the X-ray powder diffraction (XRPD) pattern for crystalline compound 1, Form IX.

FIG. 6 shows the X-ray powder diffraction (XRPD) pattern for crystalline compound 1, Form X.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

As used herein, a “disease or disorder associated with MK2” or, alternatively, “an MK2-mediated disease or disorder” means any disease or other deleterious condition in which MK2, or a mutant thereof, is known or suspected to play a role.

As used herein, a “disease or disorder associated with p38 MAP kinase” or, alternatively, “an p38 MAP kinase-mediated diseaseor disorder” means any disease or other deleterious condition in which p38 MAP kinase, or a mutant thereof, is known or suspected to play a role.

The term “substantially the same as” as used herein, refers to a powder X-ray diffraction pattern, DSC thermogram, or TGA pattern that is identical or non-identical to those depicted herein, but that falls within the limits of experimental error, when considered by one of ordinary skill in the art.

The term “substantially similar to” as used herein, refers to a powder X-ray diffraction pattern, DSC thermogram, or TGA pattern that is non-identical to those depicted herein, and shares a majority of major peaks, which fall within the limits of experimental error, when considered by one of ordinary skill in the art.

### Compound 1

Disclosed herein is (M)-3-chloro-4-((3,5-difluoropyridin-2-ylimethoxy-d2)-3′-fluoro-2′-(3-(2-hydroxypropan-2-yl)-1H-pyrazol-1-yl)-5′,6-dimethyl-2H-[1,4′-bipyridin]-2-one (compound 1), or a pharmaceutically acceptable salt of solvate thereof. Compound 1 refers to the compound with the following formula:

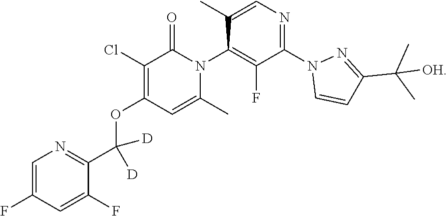

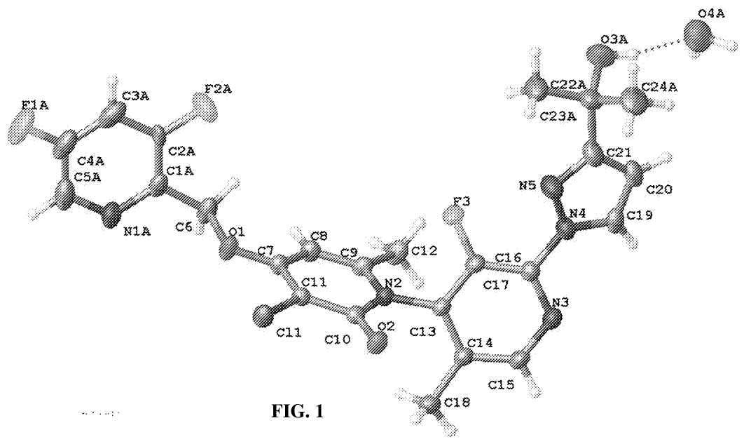

Disclosed herein is (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1), or a pharmaceutically acceptable solvate thereof. Disclosed herein is (M)-3-chloro-4-((3,5-difluoropyridin-2-yl)methoxy-d2)-3′-fluoro-2′-(3-(2-hydroxypropan-2-yl)-1H-pyrazol-1-yl)-5′,6- dimethyl-2H-[1,4′-bipyridin]-2-one (compound 1). The absolute stereochemistry of compound 1 was assigned based on a single crystal structure determination study (see example 1 and FIG. 1).

The following scheme illustrates “atropisomerism” with reference to compound 1. The term “atropisomerism” refers to a type of isomerism resulting from hindered rotation around a single bond due to steric strain of the substituents. This phenomenon creates stereoisomers which display axial chirality.

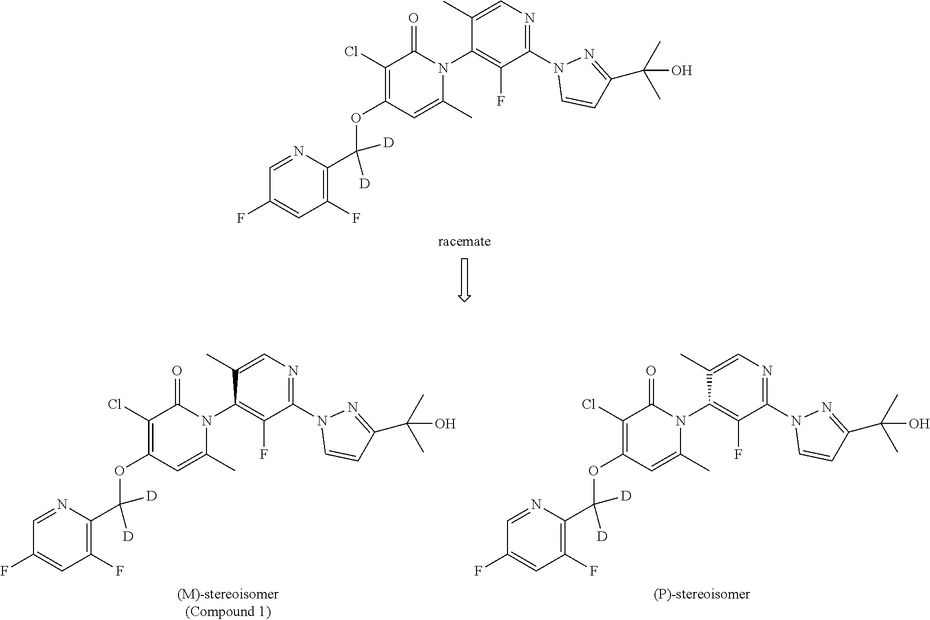

The bond between the pyridine and pyridone rings of the title compound is hindered and does not allow for facile rotation. The steric strain barrier to rotation is sufficiently high such that individual conformers can be isolated.

Atropisomers are generally stable but can often be equilibrated thermally. Atropisomers will have the same but opposite optical rotation. Each atropisomers may have different properties when bound to an enzyme or receptor with one isomer often being more potent than the other. Atropisomers are frequently used as pharmaceutical agents. Known examples include Vancomycin and derivatives.

The configuration of atropisomers can be described using the nomenclature (M)- and (P)- to describe the relative position of substituents as described in Bringmann, G. et. al., Angew. Chem. Int. Ed. 2005, 44, 5384 and references cited therein. Structures are designated as drawn but it is understood that either (P)- or (M)-isomers may be desirable and the methods described would be useful for the interconversion of either (P)- or (M)-stereoisomers.

The term “interconversion” or “conformational interconversion” refers to any change between the atropisomers of this disclosure, including but not limited to equilibration. The term “equilibration” refers to a chemical reaction in which the forward and reverse ratio rates cancel out. Equilibration can be dynamic or static. A reaction in equilibrium need not contain equal parts reactant and product.

In some embodiments, compound 1 is a freebase.

In some embodiments, compound 1 is a solvate. In some embodiments, compound 1 is a hydrate. In some embodiments, compound 1 is unsolvated. In some embodiments, compound 1 is anhydrous.

In other embodiments, compound 1 is prepared in various forms, including but not limited to, an amorphous phase, crystalline forms, milled forms, and nano-particulate forms.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

### Crystalline Forms

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### Crystalline Forms of Compound 1

The polymorphs made according to the methods of the invention may be characterized by any methodology according to the art. For example, the polymorphs made according to the methods of the invention may be characterized by X-ray powder diffraction (XRPD), differential scanning calorimetry (DSC), thermogravimetric analysis (TGA), hot-stage microscopy, and/or spectroscopy (e.g., Raman, solid state nuclear magnetic resonance (ssNMR), and infrared (IR)). In some embodiments, crystallinity of a solid form is determined by X-Ray Powder Diffraction (XRPD).

XRPD: Polymorphs according to the invention may be characterized by XRPD. The relative intensities of XRPD peaks can vary, depending upon the particle size, the sample preparation technique, the sample mounting procedure and the particular instrument employed. Moreover, instrument variation and other factors can affect the 2θ values. Therefore, the XRPD peak assignments can vary, for example by plus or minus 0.2 degrees.

DSC: Polymorphs according to the invention can also be identified by its characteristic DSC thermograms. For DSC, it is known that the temperatures observed will depend upon the rate of temperature change as well as sample preparation technique and the particular instrument employed. Thus, the values reported herein relating to DSC thermograms can vary, for example by plus or minus 4° C.

TGA: The polymorphic forms of the invention may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of compound 1 are useful in the production of medicinal preparations and can be obtained by means of a crystallization process to produce crystalline and semi-crystalline forms or a solidification process to obtain the amorphous form. In some embodiments, the crystallization is carried out by either generating the desired compound (for example, compound 1) in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. In some embodiments, the crystallization comprises addition of a seed form of a desired polymorph. The crystallization or solidification may be followed by drying carried out under controlled conditions until the desired water content is reached in the end polymorphic form.

### Polymorph Form I of Compound 1

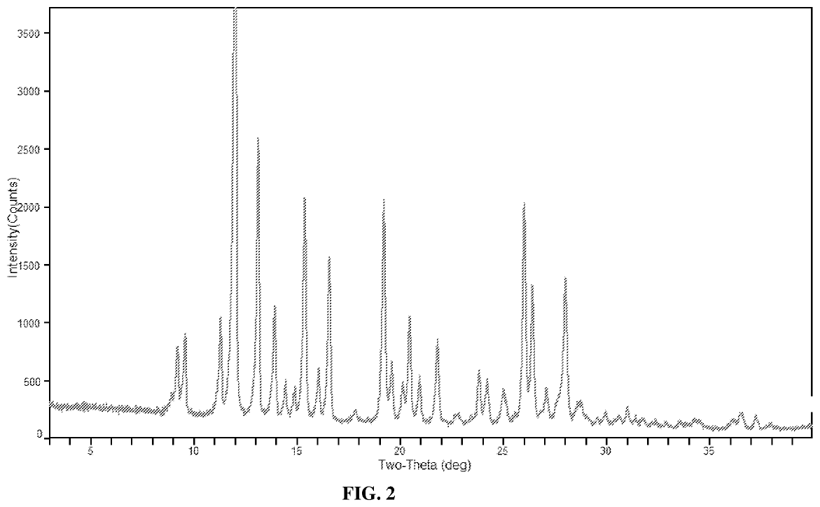

In some embodiments, compound 1 is crystalline. In some embodiments, crystalline compound 1 is Form I characterized as having at least one of the following properties:
(a) an X-ray powder diffraction (XRPD) pattern substantially the same as shown in FIG. 2;
(b) an X-ray powder diffraction (XRPD) pattern with characteristic peaks at 12.02±0.2° 2θ, 13.13±0.2° 2θ, 16.56±0.2° 2θ, 19.20±0.2° 2θ, 26.00±0.2° 2θ, and 28.00±0.2° 2θ;
(c) a DSC thermogram with an endotherm having an onset temperature at about 27° C. and a peak temperature at about 62° C.;
(d) a DSC thermogram with an endotherm having an onset temperature at about 98° C. and a peak temperature at about 104° C.;
(e) a thermogravimetric analysis (TGA) thermogram comprising a loss in mass of about 2.6% over a temperature range of about 25° C. to about 90° C.; or
(f) combinations thereof.

In some embodiments, crystalline compound 1, Form I is characterized as having at least one of the properties selected from (a) to (e). In some embodiments, crystalline compound 1, Form I is characterized as having at least two of the properties selected from (a) to (e). In some embodiments, crystalline compound 1, Form I is characterized as having at least three of the properties selected from (a) to (e). In some embodiments, crystalline compound 1, Form I is characterized as having at least four of the properties selected from (a) to (e). In some embodiments, crystalline compound 1, Form I is characterized as having properties (a) to (e).

In some embodiments, crystalline compound 1, Form I has an X-ray powder diffraction (XRPD) pattern substantially the same as shown in FIG. 2. In some embodiments, crystalline compound 1, Form I has an X-ray powder diffraction (XRPD) pattern with characteristic peaks found in Table 1. In some embodiments, crystalline compound 1, Form I has an X-ray powder diffraction (XRPD) pattern with characteristic peaks at 12.02±0.2° 2θ, 13.13±0.2° 2θ, 15.37±0.2° 2θ; 16.56±0.2° 2θ, 19.20±0.2° 2θ, 26.00±0.2° 2θ, and 28.00±0.2° 2θ.

In some embodiments, crystalline compound 1, Form I has an X-ray powder diffraction (XRPD) pattern with at least three characteristic peaks selected from 12.02±0.2° 2θ, 13.13±0.2° 2θ, 15.37±0.2° 2θ; 16.56±0.2° 2θ, 19.20±0.2° 2θ, 26.00±0.2° 2θ, and 28.00±0.2° 2θ.

In some embodiments, crystalline compound 1, Form I has an X-ray powder diffraction (XRPD) pattern with at least four characteristic peaks selected from 12.02±0.2° 2θ, 13.13±0.2° 2θ, 15.37±0.2° 2θ; 16.56±0.2° 2θ, 19.20±0.2° 2θ, 26.00±0.2° 2θ, and 28.00±0.2° 2θ.

In some embodiments, crystalline compound 1, Form I has an X-ray powder diffraction (XRPD) pattern with at least five characteristic peaks selected from 12.02±0.2° 2θ, 13.13±0.2° 2θ, 15.37±0.2° 2θ; 16.56±0.2° 2θ, 19.20±0.2° 2θ, 26.00±0.2° 2θ, and 28.00±0.2° 2θ.

In some embodiments, the X-ray powder diffraction pattern further comprises at least one peak selected from 11.30±0.2° 2θ, 13.89±0.2° 2θ, 20.45±0.2° 2θ, and 26.39±0.2° 2θ.

In some embodiments, the X-ray powder diffraction pattern further comprises at least two peaks selected from 11.30±0.2° 2θ, 13.89±0.2° 2θ, 20.45±0.2° 2θ, and 26.39±0.2° 2θ.

In some embodiments, the X-ray powder diffraction pattern further comprises at least three peaks selected from 11.30±0.2° 2θ, 13.89±0.2° 2θ, 20.45±0.2° 2θ, and 26.39±0.2° 2θ.

In some embodiments, the X-ray powder diffraction pattern further comprises peaks at 11.30±0.2° 2θ, 13.89±0.2° 2θ, 20.45±0.2° 2θ, and 26.39±0.2° 2θ.

In some embodiments, crystalline compound 1, Form I has an X-ray powder diffraction (XRPD) pattern with characteristic peaks at 11.30±0.2° 2θ, 12.02±0.2° 2θ, 13.13±0.2° 2θ, 13.89±0.2° 2θ, 15.37±0.2° 2θ; 16.56±0.2° 2θ, 19.20±0.2° 2θ, 20.45±0.2° 2θ, 26.00±0.2° 2θ, 26.39±0.2° 2θ, and 28.00±0.2° 2θ.

In some embodiments, crystalline compound 1, Form I has an X-ray powder diffraction (XRPD) pattern with at least five characteristic peaks selected from 11.30±0.2° 2θ, 12.02±0.2° 2θ, 13.13±0.2° 2θ, 13.89±0.2° 2θ, 15.37±0.2° 2θ; 16.56±0.2° 2θ, 19.20±0.2° 2θ, 20.45±0.2° 2θ, 26.00±0.2° 2θ, 26.39±0.2° 2θ, and 28.00±0.2° 2θ.

In some embodiments, crystalline compound 1, Form I has an X-ray powder diffraction (XRPD) pattern with at least six characteristic peaks selected from 11.30±0.2° 2θ, 12.02±0.2° 2θ, 13.13±0.2° 2θ, 13.89±0.2° 2θ, 15.37±0.2° 2θ; 16.56±0.2° 2θ, 19.20±0.2° 2θ, 20.45±0.2° 2θ, 26.00±0.2° 2θ, 26.39±0.2° 2θ, and 28.00±0.2° 2θ.

In some embodiments, crystalline compound 1, Form I has an X-ray powder diffraction (XRPD) pattern with at least seven characteristic peaks selected from 11.30±0.2° 2θ, 12.02±0.2° 2θ, 13.13±0.2° 2θ, 13.89±0.2° 2θ, 15.37±0.2° 2θ; 16.56±0.2° 2θ, 19.20±0.2° 2θ, 20.45±0.2° 2θ, 26.00±0.2° 2θ, 26.39±0.2° 2θ, and 28.00±0.2° 2θ.

In some embodiments, crystalline compound 1, Form I has an X-ray powder diffraction (XRPD) pattern with at least eight characteristic peaks selected from 11.30±0.2° 2θ, 12.02±0.2° 2θ, 13.13±0.2° 2θ, 13.89±0.2° 2θ, 15.37±0.2° 2θ; 16.56±0.2° 2θ, 19.20±0.2° 2θ, 20.45±0.2° 2θ, 26.00±0.2° 2θ, 26.39±0.2° 2θ, and 28.00±0.2° 2θ.

In some embodiments, the X-ray powder diffraction pattern further comprises at least one peak selected from 9.21±0.2° 2θ, 9.57±0.2° 2θ, and 21.81±0.2° 2θ.

In some embodiments, crystalline compound 1, Form I has a DSC thermogram with an endotherm having an onset temperature at about 27° C. and a peak temperature at about 62° C.

In some embodiments, crystalline compound 1, Form I has a DSC thermogram with an endotherm having an onset temperature at about 98° C. and a peak temperature at about 104° C.

In some embodiments, crystalline compound 1, Form I has a DSC thermogram with an endotherm having an onset temperature at about 27° C. and a peak temperature at about 62° C. and an onset temperature at about 98° C. and a peak temperature at about 104° C.

In some embodiments, crystalline compound 1, Form I has a thermogravimetric analysis (TGA) thermogram comprising a loss in mass of about 2.6% over a temperature range of about 25° C. to about 90° C.

In some embodiments, crystalline compound 1, Form I is a hydrate.

TABLE 1
Form I
Form I

| 2-Theta | d(Å) | BG | Height | I % | Area | I % | FWHM |
|---|---|---|---|---|---|---|---|
| 5.75 | 15.3582 | 248 | 58 | 0.7 | 576 | 0.6 | 0.111 |
| 8.922 | 9.9036 | 217 | 175 | 2.2 | 7080 | 7.2 | 0.452 |
| 9.21 | 9.5937 | 213 | 582 | 7.4 | 15079 | 15.3 | 0.289 |
| 9.566 | 9.2383 | 212 | 700 | 9 | 10628 | 10.8 | 0.169 |
| 11.298 | 7.8255 | 228 | 828 | 10.6 | 13488 | 13.7 | 0.182 |
| 12.019 | 7.3574 | 245 | 7819 | 100 | 98289 | 100 | 0.14 |
| 13.123 | 6.7409 | 224 | 2377 | 30.4 | 30579 | 31.1 | 0.144 |
| 13.887 | 6.3719 | 214 | 928 | 11.9 | 13830 | 14.1 | 0.166 |
| 14.437 | 6.1304 | 211 | 291 | 3.7 | 3114 | 3.2 | 0.119 |
| 14.883 | 5.9475 | 207 | 247 | 3.2 | 3134 | 3.2 | 0.142 |
| 15.369 | 5.7604 | 205 | 1874 | 24 | 26767 | 27.2 | 0.159 |
| 16.038 | 5.5217 | 191 | 419 | 5.4 | 5401 | 5.5 | 0.144 |
| 16.563 | 5.3478 | 180 | 1388 | 17.8 | 17804 | 18.1 | 0.143 |
| 17.799 | 4.9792 | 150 | 93 | 1.2 | 1708 | 1.7 | 0.205 |
| 19.203 | 4.6182 | 159 | 1911 | 24.4 | 30534 | 31.1 | 0.178 |
| 19.584 | 4.5292 | 169 | 497 | 6.4 | 8456 | 8.6 | 0.19 |
| 20.109 | 4.412 | 176 | 312 | 4 | 5868 | 6 | 0.21 |
| 20.45 | 4.3392 | 164 | 899 | 11.5 | 14407 | 14.7 | 0.179 |
| 20.948 | 4.2372 | 182 | 361 | 4.6 | 3772 | 3.8 | 0.117 |
| 21.803 | 4.073 | 133 | 720 | 9.2 | 11005 | 11.2 | 0.171 |
| 22.825 | 3.8929 | 124 | 98 | 1.3 | 2075 | 2.1 | 0.236 |
| 23.423 | 3.7948 | 132 | 64 | 0.8 | 1360 | 1.4 | 0.237 |
| 23.811 | 3.7338 | 129 | 466 | 6 | 9048 | 9.2 | 0.217 |
| 24.193 | 3.6757 | 133 | 378 | 4.8 | 6668 | 6.8 | 0.197 |
| 25.007 | 3.5578 | 148 | 280 | 3.6 | 5584 | 5.7 | 0.223 |
| 26.004 | 3.4237 | 178 | 1865 | 23.9 | 29958 | 30.5 | 0.179 |
| 26.386 | 3.375 | 176 | 1149 | 14.7 | 19647 | 20 | 0.191 |
| 27.081 | 3.2899 | 194 | 239 | 3.1 | 3367 | 3.4 | 0.157 |
| 28 | 3.184 | 183 | 1208 | 15.4 | 23541 | 24 | 0.217 |
| 28.591 | 3.1195 | 163 | 150 | 1.9 | 5168 | 5.3 | 0.385 |
| 28.762 | 3.1013 | 153 | 160 | 2 | 5149 | 5.2 | 0.359 |
| 29.573 | 3.0181 | 124 | 56 | 0.7 | 812 | 0.8 | 0.162 |
| 29.944 | 2.9816 | 130 | 104 | 1.3 | 1221 | 1.2 | 0.131 |
| 30.599 | 2.9192 | 136 | 66 | 0.8 | 946 | 1 | 0.16 |
| 31.019 | 2.8807 | 123 | 156 | 2 | 2594 | 2.6 | 0.186 |
| 31.401 | 2.8465 | 122 | 77 | 1 | 1161 | 1.2 | 0.168 |
| 31.74 | 2.8168 | 124 | 48 | 0.6 | 728 | 0.7 | 0.169 |
| 32.375 | 2.763 | 102 | 62 | 0.8 | 890 | 0.9 | 0.16 |
| 33.55 | 2.6689 | 102 | 57 | 0.7 | 562 | 0.6 | 0.11 |
| 34.251 | 2.6159 | 87 | 85 | 1.1 | 3704 | 3.8 | 0.486 |
| 36.076 | 2.4876 | 82 | 87 | 1.1 | 2532 | 2.6 | 0.325 |
| 36.471 | 2.4616 | 80 | 182 | 2.3 | 5533 | 5.6 | 0.339 |
| 37.241 | 2.4124 | 85 | 119 | 1.5 | 1573 | 1.6 | 0.148 |
| 37.967 | 2.368 | 79 | 58 | 0.7 | 1065 | 1.1 | 0.205 |

### Polymorph Form II of Compound 1

In some embodiments, compound 1 is crystalline. In some embodiments, crystalline compound 1 is Form II characterized as having at least one of the following properties:
(a) an X-ray powder diffraction (XRPD) pattern with characteristic peaks at 5.89±0.2° 2θ, 8.02±0.2° 2θ, 11.88±0.2° 2θ, 12.39±0.2° 2θ, 14.47±0.2° 2θ, 18.11±0.2° 2θ, and 19.86±0.2° 2θ.
(b) a DSC thermogram with an endotherm having an onset temperature at about 103° C. and a peak temperature at about 116° C.;
(c) a DSC thermogram with an endotherm having an onset temperature at about 148° C. and a peak temperature at about 155° C.;
(d) a thermogravimetric analysis (TGA) thermogram comprising a loss in mass of about 1% over a temperature range of about 90° C. to about 130° C.; or
(e) combinations thereof.

In some embodiments, crystalline compound 1, Form II is characterized as having at least one of the properties selected from (a) to (d). In some embodiments, crystalline compound 1, Form II is characterized as having at least two of the properties selected from (a) to (d). In some embodiments, crystalline compound 1, Form II is characterized as having at least three of the properties selected from (a) to (d). In some embodiments, crystalline compound 1, Form II is characterized as having properties (a) to (d).

In some embodiments, crystalline compound 1, Form II has an X-ray powder diffraction (XRPD) pattern with characteristic peaks found in Table 2. In some embodiments, crystalline compound 1, Form II has an X-ray powder diffraction (XRPD) pattern with characteristic peaks at 5.89±0.2° 2θ, 8.02±0.2° 2θ, 11.88±0.2° 2θ, 12.39±0.2° 2θ, 14.47±0.2° 2θ, 18.11±0.2° 2θ, and 19.86±0.2° 2θ.

In some embodiments, crystalline compound 1, Form II has an X-ray powder diffraction (XRPD) pattern with at least three characteristic peaks selected from 5.89±0.2° 2θ, 8.02±0.2° 2θ, 11.88±0.2° 2θ, 12.39±0.2° 2θ, 14.47±0.2° 2θ, 18.11±0.2° 2θ, and 19.86±0.2° 2θ.

In some embodiments, crystalline compound 1, Form II has an X-ray powder diffraction (XRPD) pattern with at least four characteristic peaks selected from 5.89±0.2° 2θ, 8.02±0.2° 2θ, 11.88±0.2° 2θ, 12.39±0.2° 2θ, 14.47±0.2° 2θ, 18.11±0.2° 2θ, and 19.86±0.2° 2θ.

In some embodiments, crystalline compound 1, Form II has an X-ray powder diffraction (XRPD) pattern with at least five characteristic peaks selected from 5.89±0.2° 2θ, 8.02±0.2° 2θ, 11.88±0.2° 2θ, 12.39±0.2° 2θ, 14.47±0.2° 2θ, 18.11±0.2° 2θ, and 19.86±0.2° 2θ.

In some embodiments, the X-ray powder diffraction pattern further comprises at least one peak selected from 9.03±0.2° 2θ, 12.06±0.2° 2θ, 12.97±0.2° 2θ, 15.28±0.2° 2θ, 20.48±0.2° 2θ, and 24.28±0.2° 2θ.

In some embodiments, the X-ray powder diffraction pattern further comprises at least two peaks selected from 9.03±0.2° 2θ, 12.06±0.2° 2θ, 12.97±0.2° 2θ, 15.28±0.2° 2θ, 20.48±0.2° 2θ, and 24.28±0.2° 2θ.

In some embodiments, the X-ray powder diffraction pattern further comprises at least three peaks selected from 9.03±0.2° 2θ, 12.06±0.2° 2θ, 12.97±0.2° 2θ, 15.28±0.2° 2θ, 20.48±0.2° 2θ, and 24.28±0.2° 2θ.

In some embodiments, the X-ray powder diffraction pattern further comprises peaks at 9.03±0.2° 2θ, 12.06±0.2° 2θ, 12.97±0.2° 2θ, 15.28±0.2° 2θ, 20.48±0.2° 2θ, and 24.28±0.2° 2θ.

In some embodiments, crystalline compound 1, Form II has an X-ray powder diffraction (XRPD) pattern with characteristic peaks at 5.89±0.2° 2θ, 8.02±0.2° 2θ, 9.03±0.2° 2θ, 11.88±0.2° 2θ, 12.06±0.2° 2θ, 12.39±0.2° 2θ, 12.97±0.2° 2θ, 14.47±0.2° 2θ, 15.28±0.2° 2θ, 18.11±0.2° 2θ, 19.86±0.2° 2θ, 20.48±0.2° 2θ, and 24.28±0.2° 2θ.

In some embodiments, crystalline compound 1, Form II has an X-ray powder diffraction (XRPD) pattern with at least five characteristic peaks selected from 5.89±0.2° 2θ, 8.02±0.2° 2θ, 9.03±0.2° 2θ, 11.88±0.2° 2θ, 12.06±0.2° 2θ, 12.39±0.2° 2θ, 12.97±0.2° 2θ, 14.47±0.2° 2θ, 15.28±0.2° 2θ, 18.11±0.2° 2θ, 19.86±0.2° 2θ, 20.48±0.2° 2θ, and 24.28±0.2° 2θ.

In some embodiments, crystalline compound 1, Form II has an X-ray powder diffraction (XRPD) pattern with at least six characteristic peaks selected from 5.89±0.2° 2θ, 8.02±0.2° 2θ, 9.03±0.2° 2θ, 11.88±0.2° 2θ, 12.06±0.2° 2θ, 12.39±0.2° 2θ, 12.97±0.2° 2θ, 14.47±0.2° 2θ, 15.28±0.2° 2θ, 18.11±0.2° 2θ, 19.86±0.2° 2θ, 20.48±0.2° 2θ, and 24.28±0.2° 2θ.

In some embodiments, crystalline compound 1, Form II has an X-ray powder diffraction (XRPD) pattern with at least seven characteristic peaks selected from 5.89±0.2° 2θ, 8.02±0.2° 2θ, 9.03±0.2° 2θ, 11.88±0.2° 2θ, 12.06±0.2° 2θ, 12.39±0.2° 2θ, 12.97±0.2° 2θ, 14.47±0.2° 2θ, 15.28±0.2° 2θ, 18.11±0.2° 2θ, 19.86±0.2° 2θ, 20.48±0.2° 2θ, and 24.28±0.2° 2θ.

In some embodiments, crystalline compound 1, Form II has an X-ray powder diffraction (XRPD) pattern with at least eight characteristic peaks selected from 5.89±0.2° 2θ, 8.02±0.2° 2θ, 9.03±0.2° 2θ, 11.88±0.2° 2θ, 12.06±0.2° 2θ, 12.39±0.2° 2θ, 12.97±0.2° 2θ, 14.47±0.2° 2θ, 15.28±0.2° 2θ, 18.11±0.2° 2θ, 19.86±0.2° 2θ, 20.48±0.2° 2θ, and 24.28±0.2° 2θ.

In some embodiments, the X-ray powder diffraction pattern further comprises at least one peak selected from 13.54±0.2° 2θ, 16.10±0.2° 2θ, and 21.71±0.2° 2θ.

In some embodiments, crystalline compound 1, Form II has a DSC thermogram with an endotherm having an onset temperature at about 103° C. and a peak temperature at about 116° C.

In some embodiments, crystalline compound 1, Form II has a DSC thermogram with an endotherm having an onset temperature at about 148° C. and a peak temperature at about 155° C.

In some embodiments, crystalline compound 1, Form II has a DSC thermogram with an endotherm having an onset temperature at about 103° C. and a peak temperature at about 116° C. and an onset temperature at about 148° C. and a peak temperature at about 155° C.

In some embodiments, crystalline compound 1, Form II has a thermogravimetric analysis (TGA) thermogram comprising a loss in mass of about 1% over a temperature range of about 90° C. to about 130° C.

In some embodiments, crystalline compound 1, Form II is a solvate. In some embodiments, crystalline compound 1, Form II is a EtOH solvate. In some embodiments, crystalline compound 1, Form II is a IPA solvate.

TABLE 2
Form II

| 2-Theta | d(Å) | BG | Height | I % | Area | I % | FWHM |
|---|---|---|---|---|---|---|---|
| 4.036 | 21.8725 | 297 | 115 | 16.2 | 1915 | 12 | 0.186 |
| 4.511 | 19.5725 | 316 | 80 | 11.2 | 1480 | 9.3 | 0.206 |
| 5.887 | 14.9994 | 318 | 712 | 100 | 15922 | 100 | 0.25 |
| 6.428 | 13.7387 | 319 | 118 | 16.6 | 2155 | 13.5 | 0.204 |
| 8.016 | 11.0197 | 273 | 669 | 94 | 8996 | 56.5 | 0.15 |
| 9.026 | 9.7893 | 256 | 250 | 35.1 | 3656 | 23 | 0.163 |
| 10.273 | 8.6034 | 233 | 56 | 7.9 | 260 | 1.6 | 0.052 |
| 11.876 | 7.4457 | 239 | 590 | 82.9 | 11145 | 70 | 0.211 |
| 12.06 | 7.3327 | 245 | 248 | 34.8 | 9405 | 59.1 | 0.423 |
| 12.389 | 7.1384 | 224 | 516 | 72.5 | 8977 | 56.4 | 0.194 |
| 12.966 | 6.8221 | 247 | 237 | 33.3 | 3065 | 19.3 | 0.144 |
| 13.546 | 6.5314 | 210 | 186 | 26.1 | 2287 | 14.4 | 0.137 |
| 14.476 | 6.1137 | 197 | 374 | 52.5 | 6215 | 39 | 0.185 |
| 15.277 | 5.7949 | 212 | 281 | 39.5 | 6577 | 41.3 | 0.261 |
| 15.894 | 5.5713 | 192 | 114 | 16 | 4549 | 28.6 | 0.445 |
| 16.105 | 5.4988 | 209 | 167 | 23.5 | 3125 | 19.6 | 0.209 |
| 16.899 | 5.2422 | 173 | 64 | 9 | 547 | 3.4 | 0.095 |
| 18.113 | 4.8936 | 166 | 629 | 88.3 | 13761 | 86.4 | 0.244 |
| 19.445 | 4.5611 | 224 | 68 | 9.6 | 275 | 1.7 | 0.045 |
| 19.859 | 4.467 | 169 | 412 | 57.9 | 12562 | 78.9 | 0.34 |
| 20.477 | 4.3336 | 183 | 332 | 46.6 | 8182 | 51.4 | 0.275 |
| 21.711 | 4.09 | 136 | 177 | 24.9 | 4239 | 26.6 | 0.267 |
| 22.683 | 3.9169 | 133 | 110 | 15.4 | 1683 | 10.6 | 0.171 |
| 23.865 | 3.7255 | 124 | 116 | 16.3 | 3275 | 20.6 | 0.315 |
| 24.284 | 3.6621 | 119 | 269 | 37.8 | 7246 | 45.5 | 0.301 |
| 24.966 | 3.5637 | 105 | 80 | 11.2 | 815 | 5.1 | 0.114 |
| 26.11 | 3.41 | 90 | 61 | 8.6 | 1241 | 7.8 | 0.227 |
| 27.381 | 3.2546 | 88 | 56 | 7.9 | 1756 | 11 | 0.35 |
| 27.642 | 3.2244 | 88 | 44 | 6.2 | 1754 | 11 | 0.445 |
| 28.743 | 3.1033 | 84 | 48 | 6.7 | 1591 | 10 | 0.37 |
| 29.51 | 3.0244 | 86 | 46 | 6.5 | 590 | 3.7 | 0.143 |
| 30.664 | 2.9132 | 62 | 140 | 19.7 | 4056 | 25.5 | 0.323 |
| 32.187 | 2.7787 | 58 | 50 | 7 | 949 | 6 | 0.212 |
| 34.146 | 2.6236 | 50 | 35 | 4.9 | 747 | 4.7 | 0.238 |

### Polymorph Form III of Compound 1

In some embodiments, compound 1 is crystalline. In some embodiments, crystalline compound 1 is Form III characterized as having at least one of the following properties:
(a) an X-ray powder diffraction (XRPD) pattern with characteristic peaks at 5.23±0.2° 2θ, 7.53±0.2° 2θ, 10.39±0.2° 2θ, 14.47±0.2° 2θ, 17.23±0.2° 2θ, 18.41±0.2° 2θ, and 19.16±0.2° 2θ.
(b) a DSC thermogram with an endotherm having an onset temperature at about 78° C. and a peak temperature at about 85° C.;
(c) a thermogravimetric analysis (TGA) thermogram comprising a loss in mass of about 9.2% over a temperature range of about 50° C. to about 135° C.; or
(d) combinations thereof.

In some embodiments, crystalline compound 1, Form III is characterized as having at least one of the properties selected from (a) to (c). In some embodiments, crystalline compound 1, Form III is characterized as having at least two of the properties selected from (a) to (c). In some embodiments, crystalline compound 1, Form III is characterized as having properties (a) to (c).

In some embodiments, crystalline compound 1, Form III has an X-ray powder diffraction (XRPD) pattern with characteristic peaks found in Table 3. In some embodiments, crystalline compound 1, Form III has an X-ray powder diffraction (XRPD) pattern with characteristic peaks at 5.23±0.2° 2θ, 7.53±0.2° 2θ, 10.39±0.2° 2θ, 14.47±0.2° 2θ, 17.23±0.2° 2θ, 18.41±0.2° 2θ, and 19.16±0.2° 2θ.

In some embodiments, crystalline compound 1, Form III has an X-ray powder diffraction (XRPD) pattern with at least three characteristic peaks selected from 5.23±0.2° 2θ, 7.53±0.2° 2θ, 10.39±0.2° 2θ, 14.47±0.2° 2θ, 17.23±0.2° 2θ, 18.41±0.2° 2θ, and 19.16±0.2° 2θ.

In some embodiments, crystalline compound 1, Form III has an X-ray powder diffraction (XRPD) pattern with at least four characteristic peaks selected from 5.23±0.2° 2θ, 7.53±0.2° 2θ, 10.39±0.2° 2θ, 14.47±0.2° 2θ, 17.23±0.2° 2θ, 18.41±0.2° 2θ, and 19.16±0.2° 2θ.

In some embodiments, crystalline compound 1, Form III has an X-ray powder diffraction (XRPD) pattern with at least five characteristic peaks selected from 5.23±0.2° 2θ, 7.53±0.2° 2θ, 10.39±0.2° 2θ, 14.47±0.2° 2θ, 17.23±0.2° 2θ, 18.41±0.2° 2θ, and 19.16±0.2° 2θ.

In some embodiments, the X-ray powder diffraction pattern further comprises at least one peak selected from 11.00±0.2° 2θ, 14.85±0.2° 2θ, 21.39±0.2° 2θ, 22.63±0.2° 2θ, and 25.25±0.2° 2θ.

In some embodiments, the X-ray powder diffraction pattern further comprises at least two peaks selected from 11.00±0.2° 2θ, 14.85±0.2° 2θ, 21.39±0.2° 2θ, 22.63±0.2° 2θ, and 25.25±0.2° 2θ.

In some embodiments, the X-ray powder diffraction pattern further comprises at least three peaks selected from 11.00±0.2° 2θ, 14.85±0.2° 2θ, 21.39±0.2° 2θ, 22.63±0.2° 2θ, and 25.25±0.2° 2θ.

In some embodiments, the X-ray powder diffraction pattern further comprises peaks at 11.00±0.2° 2θ, 14.85±0.2° 2θ, 21.39±0.2° 2θ, 22.63±0.2° 2θ, and 25.25±0.2° 2θ.

In some embodiments, crystalline compound 1, Form III has an X-ray powder diffraction (XRPD) pattern with characteristic peaks at 5.23±0.2° 2θ, 7.53±0.2° 2θ, 10.39±0.2° 2θ, 11.00±0.2° 2θ, 14.47±0.2° 2θ, 14.85±0.2° 2θ, 17.23±0.2° 2θ, 18.41±0.2° 2θ, 19.16±0.2° 2θ, 21.39±0.2° 2θ, 22.63±0.2° 2θ, and 25.25±0.2° 2θ.

In some embodiments, crystalline compound 1, Form III has an X-ray powder diffraction (XRPD) pattern with at least five characteristic peaks selected from 5.23±0.2° 2θ, 7.53±0.2° 2θ, 10.39±0.2° 2θ, 11.00±0.2° 2θ, 14.47±0.2° 2θ, 14.85±0.2° 2θ, 17.23±0.2° 2θ, 18.41±0.2° 2θ, 19.16±0.2° 2θ, 21.39±0.2° 2θ, 22.63±0.2° 2θ, and 25.25±0.2° 2θ.

In some embodiments, crystalline compound 1, Form III has an X-ray powder diffraction (XRPD) pattern with at least six characteristic peaks selected from 5.23±0.2° 2θ, 7.53±0.2° 2θ, 10.39±0.2° 2θ, 11.00±0.2° 2θ, 14.47±0.2° 2θ, 14.85±0.2° 2θ, 17.23±0.2° 2θ, 18.41±0.2° 2θ, 19.16±0.2° 2θ, 21.39±0.2° 2θ, 22.63±0.2° 2θ, and 25.25±0.2° 2θ.

In some embodiments, crystalline compound 1, Form III has an X-ray powder diffraction (XRPD) pattern with at least seven characteristic peaks selected from 5.23±0.2° 2θ, 7.53±0.2° 2θ, 10.39±0.2° 2θ, 11.00±0.2° 2θ, 14.47±0.2° 2θ, 14.85±0.2° 2θ, 17.23±0.2° 2θ, 18.41±0.2° 2θ, 19.16±0.2° 2θ, 21.39±0.2° 2θ, 22.63±0.2° 2θ, and 25.25±0.2° 2θ.

In some embodiments, crystalline compound 1, Form III has an X-ray powder diffraction (XRPD) pattern with at least eight characteristic peaks selected from 5.23±0.2° 2θ, 7.53±0.2° 2θ, 10.39±0.2° 2θ, 11.00±0.2° 2θ, 14.47±0.2° 2θ, 14.85±0.2° 2θ, 17.23±0.2° 2θ, 18.41±0.2° 2θ, 19.16±0.2° 2θ, 21.39±0.2° 2θ, 22.63±0.2° 2θ, and 25.25±0.2° 2θ.

In some embodiments, the X-ray powder diffraction pattern further comprises at least one peak selected from 24.16±0.2° 2θ, 25.99±0.2° 2θ, and 29.21±0.2° 2θ.

In some embodiments, crystalline compound 1, Form III has a DSC thermogram with an endotherm having an onset temperature at about 78° C. and a peak temperature at about 85° C.

In some embodiments, crystalline compound 1, Form III has a thermogravimetric analysis (TGA) thermogram comprising a loss in mass of about 9.2% over a temperature range of about 50° C. to about 135° C.

In some embodiments, crystalline compound 1, Form III is a solvate. In some embodiments, crystalline compound 1, Form III is a 2-MeTHF and CYH solvate. In some embodiments, crystalline compound 1, Form III is a 2-MeTHF solvate. In some embodiments, crystalline compound 1, Form III is a CYH solvate. In some embodiments, crystalline compound 1, Form III is a MTBE solvate.

TABLE 3
Form III

| 2-Theta | d(Å) | BG | Height | I % | Area | I % | FWHM |
|---|---|---|---|---|---|---|---|
| 5.232 | 16.8772 | 256 | 1851 | 86.1 | 39774 | 93.5 | 0.24 |
| 7.53 | 11.7306 | 230 | 2151 | 100 | 42528 | 100 | 0.221 |
| 10.392 | 8.5052 | 191 | 776 | 36.1 | 17942 | 42.2 | 0.258 |
| 11.009 | 8.0299 | 195 | 307 | 14.3 | 4867 | 11.4 | 0.177 |
| 13.596 | 6.5073 | 169 | 47 | 2.2 | 320 | 0.8 | 0.076 |
| 14.476 | 6.1138 | 196 | 841 | 39.1 | 14697 | 34.6 | 0.195 |
| 14.856 | 5.9581 | 170 | 406 | 18.9 | 17706 | 41.6 | 0.487 |
| 15.266 | 5.7992 | 202 | 74 | 3.4 | 2454 | 5.8 | 0.37 |
| 15.593 | 5.6782 | 198 | 94 | 4.4 | 1021 | 2.4 | 0.121 |
| 16.274 | 5.4422 | 166 | 222 | 10.3 | 3759 | 8.8 | 0.189 |
| 16.774 | 5.281 | 167 | 49 | 2.3 | 1005 | 2.4 | 0.229 |
| 17.234 | 5.141 | 172 | 1046 | 48.6 | 16416 | 38.6 | 0.175 |
| 18.415 | 4.8139 | 175 | 914 | 42.5 | 14845 | 34.9 | 0.181 |
| 19.163 | 4.6277 | 173 | 1087 | 50.5 | 17765 | 41.8 | 0.182 |
| 19.834 | 4.4727 | 170 | 195 | 9.1 | 4381 | 10.3 | 0.251 |
| 20.095 | 4.415 | 169 | 130 | 6 | 3135 | 7.4 | 0.269 |
| 20.569 | 4.3145 | 164 | 167 | 7.8 | 3684 | 8.7 | 0.246 |
| 20.844 | 4.258 | 154 | 128 | 6 | 2786 | 6.6 | 0.243 |
| 21.395 | 4.1497 | 178 | 379 | 17.6 | 5190 | 12.2 | 0.153 |
| 22.027 | 4.032 | 165 | 251 | 11.7 | 4894 | 11.5 | 0.218 |
| 22.629 | 3.926 | 168 | 286 | 13.3 | 4371 | 10.3 | 0.171 |
| 23.704 | 3.7505 | 140 | 73 | 3.4 | 679 | 1.6 | 0.104 |
| 24.166 | 3.6798 | 135 | 285 | 13.2 | 4924 | 11.6 | 0.193 |
| 24.65 | 3.6086 | 139 | 257 | 11.9 | 3727 | 8.8 | 0.162 |
| 25.256 | 3.5234 | 140 | 314 | 14.6 | 5077 | 11.9 | 0.18 |
| 25.991 | 3.4254 | 142 | 266 | 12.4 | 6615 | 15.6 | 0.278 |
| 26.384 | 3.3752 | 135 | 154 | 7.2 | 5289 | 12.4 | 0.383 |
| 26.845 | 3.3184 | 140 | 67 | 3.1 | 606 | 1.4 | 0.101 |
| 27.347 | 3.2586 | 125 | 171 | 7.9 | 5903 | 13.9 | 0.385 |
| 27.697 | 3.2181 | 116 | 234 | 10.9 | 7882 | 18.5 | 0.376 |
| 29.208 | 3.055 | 101 | 275 | 12.8 | 8877 | 20.9 | 0.36 |
| 29.496 | 3.0258 | 105 | 205 | 9.5 | 7348 | 17.3 | 0.4 |
| 30.362 | 2.9415 | 106 | 163 | 7.6 | 2264 | 5.3 | 0.155 |
| 31.44 | 2.843 | 91 | 125 | 5.8 | 4377 | 10.3 | 0.391 |
| 31.821 | 2.8099 | 90 | 79 | 3.7 | 2330 | 5.5 | 0.329 |
| 33.621 | 2.6634 | 92 | 82 | 3.8 | 2710 | 6.4 | 0.369 |
| 34.683 | 2.5843 | 82 | 79 | 3.7 | 1356 | 3.2 | 0.192 |
| 35.394 | 2.534 | 78 | 41 | 1.9 | 1442 | 3.4 | 0.393 |
| 36.601 | 2.4531 | 79 | 87 | 4 | 3473 | 8.2 | 0.446 |
| 38.043 | 2.3634 | 75 | 64 | 3 | 1101 | 2.6 | 0.192 |
| 38.795 | 2.3193 | 80 | 116 | 5.4 | 2240 | 5.3 | 0.216 |

### Polymorph Form IV of Compound 1

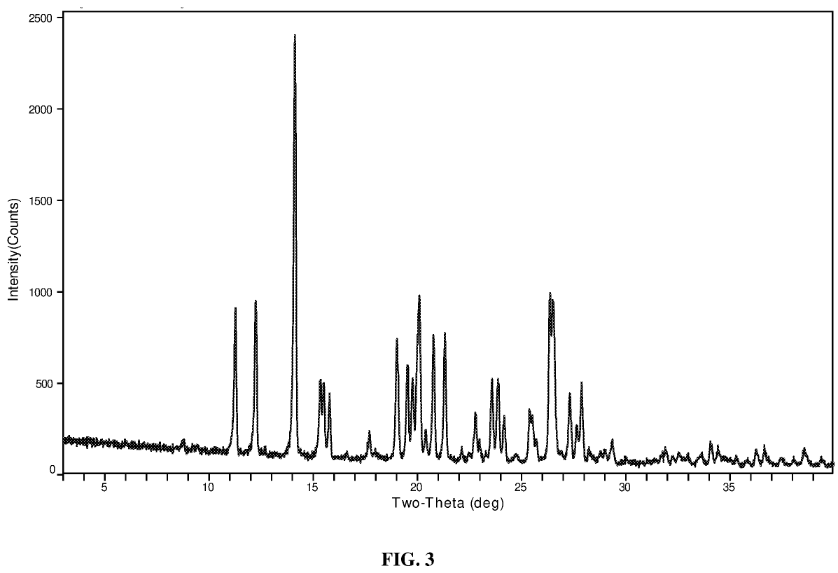

In some embodiments, compound 1 is crystalline. In some embodiments, crystalline compound 1 is Form IV characterized as having at least one of the following properties:
(a) an X-ray powder diffraction (XRPD) pattern substantially the same as shown in FIG. 3;
(b) an X-ray powder diffraction (XRPD) pattern with characteristic peaks at 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.
(c) a DSC thermogram with an endotherm having an onset temperature at about 36° C. and a peak temperature at about 52° C.;
(d) a DSC thermogram with an endotherm having an onset temperature at about 103.5° C. and a peak temperature at about 109° C.;
(e) a thermogravimetric analysis (TGA) thermogram comprising a loss in mass of about 1.5% over a temperature range of about 33° C. to about 100° C.; or
(f) combinations thereof.

In some embodiments, crystalline compound 1, Form IV is characterized as having at least one of the properties selected from (a) to (e). In some embodiments, crystalline compound 1, Form IV is characterized as having at least two of the properties selected from (a) to (e). In some embodiments, crystalline compound 1, Form IV is characterized as having at least three of the properties selected from (a) to (e). In some embodiments, crystalline compound 1, Form IV is characterized as having at least four of the properties selected from (a) to (e). In some embodiments, crystalline compound 1, Form IV is characterized as having properties (a) to (e).

In some embodiments, crystalline compound 1, Form IV has an X-ray powder diffraction (XRPD) pattern substantially the same as shown in FIG. 3. In some embodiments, crystalline compound 1, Form IV has an X-ray powder diffraction (XRPD) pattern with characteristic peaks found in Table 4. In some embodiments, crystalline compound 1, Form IV has an X-ray powder diffraction (XRPD) pattern with characteristic peaks at 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.

In some embodiments, crystalline compound 1, Form IV has an X-ray powder diffraction (XRPD) pattern with at least three characteristic peaks selected from 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.

In some embodiments, crystalline compound 1, Form IV has an X-ray powder diffraction (XRPD) pattern with at least four characteristic peaks selected from 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.

In some embodiments, crystalline compound 1, Form IV has an X-ray powder diffraction (XRPD) pattern with at least five characteristic peaks selected from 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.

In some embodiments, the X-ray powder diffraction pattern further comprises at least one peak selected from 15.35±0.2° 2θ, 19.52±0.2° 2θ, 19.78±0.2° 2θ, 23.59±0.2° 2θ, 23.86±0.2° 2θ, and 27.88±0.2° 2θ.

In some embodiments, the X-ray powder diffraction pattern further comprises at least two peaks selected from 15.35±0.2° 2θ, 19.52±0.2° 2θ, 19.78±0.2° 2θ, 23.59±0.2° 2θ, 23.86±0.2° 2θ, and 27.88±0.2° 2θ.

In some embodiments, the X-ray powder diffraction pattern further comprises at least three peaks selected from 15.35±0.2° 2θ, 19.52±0.2° 2θ, 19.78±0.2° 2θ, 23.59±0.2° 2θ, 23.86±0.2° 2θ, and 27.88±0.2° 2θ.

In some embodiments, the X-ray powder diffraction pattern further comprises peaks at 15.35±0.2° 2θ, 19.52±0.2° 2θ, 19.78±0.2° 2θ, 23.59±0.2° 2θ, 23.86±0.2° 2θ, and 27.88±0.2° 2θ.

In some embodiments, crystalline compound 1, Form IV has an X-ray powder diffraction (XRPD) pattern with characteristic peaks at 11.27±0.2° 2θ, 12.24±0.2° 2θ, 14.12±0.2° 2θ, 15.35±0.2° 2θ, 19.03±0.2° 2θ, 19.52±0.2° 2θ, 19.78±0.2° 2θ, 20.09±0.2° 2θ, 20.77±0.2° 2θ, 21.33±0.2° 2θ, 23.59±0.2° 2θ, 23.86±0.2° 2θ, 26.37±0.2° 2θ, and 27.88±0.2° 2θ.

In some embodiments, crystalline compound 1, Form IV has an X-ray powder diffraction (XRPD) pattern with at least five characteristic peaks selected from 11.27±0.2° 2θ, 12.24±0.2° 2θ, 14.12±0.2° 2θ, 15.35±0.2° 2θ, 19.03±0.2° 2θ, 19.52±0.2° 2θ, 19.78±0.2° 2θ, 20.09±0.2° 2θ, 20.77±0.2° 2θ, 21.33±0.2° 2θ, 23.59±0.2° 2θ, 23.86±0.2° 2θ, 26.37±0.2° 2θ, and 27.88±0.2° 2θ.

In some embodiments, crystalline compound 1, Form IV has an X-ray powder diffraction (XRPD) pattern with at least six characteristic peaks selected from 11.27±0.2° 2θ, 12.24±0.2° 2θ, 14.12±0.2° 2θ, 15.35±0.2° 2θ, 19.03±0.2° 2θ, 19.52±0.2° 2θ, 19.78±0.2° 2θ, 20.09±0.2° 2θ, 20.77±0.2° 2θ, 21.33±0.2° 2θ, 23.59±0.2° 2θ, 23.86±0.2° 2θ, 26.37±0.2° 2θ, and 27.88±0.2° 2θ.

In some embodiments, crystalline compound 1, Form IV has an X-ray powder diffraction (XRPD) pattern with at least seven characteristic peaks selected from 11.27±0.2° 2θ, 12.24±0.2° 2θ, 14.12±0.2° 2θ, 15.35±0.2° 2θ, 19.03±0.2° 2θ, 19.52±0.2° 2θ, 19.78±0.2° 2θ, 20.09±0.2° 2θ, 20.77±0.2° 2θ, 21.33±0.2° 2θ, 23.59±0.2° 2θ, 23.86±0.2° 2θ, 26.37±0.2° 2θ, and 27.88±0.2° 2θ.

In some embodiments, crystalline compound 1, Form IV has an X-ray powder diffraction (XRPD) pattern with at least eight characteristic peaks selected from 11.27±0.2° 2θ, 12.24±0.2° 2θ, 14.12±0.2° 2θ, 15.35±0.2° 2θ, 19.03±0.2° 2θ, 19.52±0.2° 2θ, 19.78±0.2° 2θ, 20.09±0.2° 2θ, 20.77±0.2° 2θ, 21.33±0.2° 2θ, 23.59±0.2° 2θ, 23.86±0.2° 2θ, 26.37±0.2° 2θ, and 27.88±0.2° 2θ.

In some embodiments, the X-ray powder diffraction pattern further comprises at least one peak selected from 15.51±0.2° 2θ, 15.79±0.2° 2θ, and 27.32±0.2° 2θ.

In some embodiments, crystalline compound 1, Form IV has a DSC thermogram with an endotherm having an onset temperature at about 36° C. and a peak temperature at about 52° C.

In some embodiments, crystalline compound 1, Form IV has a DSC thermogram with an endotherm having an onset temperature at about 103.5° C. and a peak temperature at about 109° C.

In some embodiments, crystalline compound 1, Form IV has a DSC thermogram with an endotherm having an onset temperature at about 36° C. and a peak temperature at about 52° C. and an onset temperature at about 103.5° C. and a peak temperature at about 109° C.

In some embodiments, crystalline compound 1, Form IV has a thermogravimetric analysis (TGA) thermogram comprising a loss in mass of about 1.5% over a temperature range of about 33° C. to about 100° C.

In some embodiments, crystalline compound 1, Form IV is a hydrate.

TABLE 4
Form IV

| 2-Theta | d(Å) | BG | Height | I % | Area | I % | FWHM |
|---|---|---|---|---|---|---|---|
| 8.805 | 10.0345 | 127 | 61 | 2.6 | 1520 | 5.7 | 0.278 |
| 11.271 | 7.8437 | 115 | 797 | 34.4 | 8732 | 32.6 | 0.122 |
| 12.244 | 7.2227 | 114 | 835 | 36.1 | 9177 | 34.3 | 0.123 |
| 12.712 | 6.9579 | 108 | 41 | 1.8 | 391 | 1.5 | 0.106 |
| 14.122 | 6.2664 | 96 | 2315 | 100 | 26781 | 100 | 0.129 |
| 15.355 | 5.7656 | 91 | 424 | 18.3 | 6948 | 25.9 | 0.183 |
| 15.512 | 5.7078 | 93 | 404 | 17.5 | 7576 | 28.3 | 0.209 |
| 15.788 | 5.6084 | 85 | 356 | 15.4 | 4662 | 17.4 | 0.146 |
| 17.706 | 5.0051 | 82 | 152 | 6.6 | 2258 | 8.4 | 0.166 |
| 17.978 | 4.93 | 83 | 54 | 2.3 | 826 | 3.1 | 0.171 |
| 19.031 | 4.6595 | 95 | 645 | 27.9 | 7551 | 28.2 | 0.131 |
| 19.519 | 4.5441 | 97 | 498 | 21.5 | 6202 | 23.2 | 0.139 |
| 19.78 | 4.4847 | 104 | 420 | 18.1 | 5931 | 22.1 | 0.158 |
| 20.096 | 4.415 | 95 | 885 | 38.2 | 15052 | 56.2 | 0.19 |
| 20.409 | 4.3479 | 122 | 118 | 5.1 | 873 | 3.3 | 0.083 |
| 20.766 | 4.2739 | 103 | 659 | 28.5 | 6651 | 24.8 | 0.113 |
| 21.329 | 4.1624 | 82 | 691 | 29.8 | 7453 | 27.8 | 0.12 |
| 22.13 | 4.0134 | 74 | 72 | 3.1 | 536 | 2 | 0.083 |
| 22.48 | 3.9517 | 77 | 40 | 1.7 | 738 | 2.8 | 0.206 |
| 22.776 | 3.901 | 80 | 255 | 11 | 3964 | 14.8 | 0.173 |
| 22.983 | 3.8665 | 82 | 108 | 4.7 | 1073 | 4 | 0.111 |
| 23.587 | 3.7687 | 78 | 442 | 19.1 | 5851 | 21.8 | 0.148 |
| 23.864 | 3.7256 | 74 | 446 | 19.3 | 6244 | 23.3 | 0.156 |
| 24.166 | 3.6797 | 72 | 245 | 10.6 | 3248 | 12.1 | 0.148 |
| 24.719 | 3.5987 | 66 | 42 | 1.8 | 676 | 2.5 | 0.18 |
| 25.399 | 3.5038 | 73 | 269 | 11.6 | 5852 | 21.9 | 0.243 |
| 25.716 | 3.4614 | 80 | 108 | 4.7 | 2095 | 7.8 | 0.216 |
| 26.371 | 3.3768 | 84 | 908 | 39.2 | 21201 | 79.2 | 0.261 |
| 26.924 | 3.3087 | 85 | 38 | 1.6 | 755 | 2.8 | 0.222 |
| 27.316 | 3.2621 | 86 | 355 | 15.3 | 4126 | 15.4 | 0.13 |
| 27.646 | 3.224 | 83 | 183 | 7.9 | 3479 | 13 | 0.212 |
| 27.882 | 3.1972 | 81 | 421 | 18.2 | 5304 | 19.8 | 0.141 |
| 28.233 | 3.1582 | 79 | 65 | 2.8 | 557 | 2.1 | 0.096 |
| 28.778 | 3.0997 | 73 | 48 | 2.1 | 1522 | 5.7 | 0.354 |
| 29.036 | 3.0727 | 66 | 64 | 2.8 | 1890 | 7.1 | 0.33 |
| 29.364 | 3.0391 | 59 | 129 | 5.6 | 1852 | 6.9 | 0.16 |
| 30 | 2.9761 | 52 | 40 | 1.7 | 717 | 2.7 | 0.2 |
| 31.768 | 2.8144 | 57 | 64 | 2.8 | 1557 | 5.8 | 0.272 |
| 31.902 | 2.8029 | 58 | 86 | 3.7 | 995 | 3.7 | 0.129 |
| 32.27 | 2.7718 | 59 | 37 | 1.6 | 501 | 1.9 | 0.151 |
| 32.543 | 2.7491 | 57 | 60 | 2.6 | 1977 | 7.4 | 0.368 |
| 33 | 2.7121 | 55 | 57 | 2.5 | 1958 | 7.3 | 0.383 |
| 33.658 | 2.6606 | 55 | 62 | 2.7 | 931 | 3.5 | 0.168 |
| 34.066 | 2.6296 | 68 | 109 | 4.7 | 987 | 3.7 | 0.101 |
| 34.422 | 2.6032 | 58 | 96 | 4.1 | 2319 | 8.7 | 0.27 |
| 35.018 | 2.5603 | 58 | 30 | 1.3 | 1398 | 5.2 | 0.52 |
| 35.296 | 2.5407 | 58 | 40 | 1.7 | 208 | 0.8 | 0.058 |
| 35.863 | 2.5019 | 46 | 39 | 1.7 | 331 | 1.2 | 0.095 |
| 36.259 | 2.4755 | 45 | 87 | 3.8 | 1510 | 5.6 | 0.194 |
| 36.653 | 2.4497 | 47 | 109 | 4.7 | 1609 | 6 | 0.165 |
| 37.504 | 2.3961 | 49 | 34 | 1.5 | 478 | 1.8 | 0.157 |
| 38.059 | 2.3624 | 48 | 44 | 1.9 | 420 | 1.6 | 0.107 |
| 38.571 | 2.3323 | 46 | 96 | 4.1 | 1812 | 6.8 | 0.211 |
| 39.383 | 2.286 | 43 | 49 | 2.1 | 586 | 2.2 | 0.133 |

### Polymorph Form VI of Compound 1

In some embodiments, compound 1 is crystalline. In some embodiments, crystalline compound 1 is Form VI characterized as having at least one of the following properties:
(a) an X-ray powder diffraction (XRPD) pattern with characteristic peaks at 4.14±0.2° 2θ, 5.90±0.2° 2θ, 9.57±0.2° 2θ, 11.49±0.2° 2θ, 17.88±0.2° 2θ, 20.20±0.2° 2θ, and 20.88±0.2° 2θ.
(b) a DSC thermogram with an endotherm having an onset temperature at about 97° C. and a peak temperature at about 108° C.;
(c) a thermogravimetric analysis (TGA) thermogram comprising a loss in mass of about 1% over a temperature range of about 70° C. to about 120° C.; or
(d) combinations thereof.

In some embodiments, crystalline compound 1, Form VI is characterized as having at least one of the properties selected from (a) to (c). In some embodiments, crystalline compound 1, Form VI is characterized as having at least two of the properties selected from (a) to (c). In some embodiments, crystalline compound 1, Form VI is characterized as having properties (a) to (c).

In some embodiments, crystalline compound 1, Form VI has an X-ray powder diffraction (XRPD) pattern with characteristic peaks found in Table 5. In some embodiments, crystalline compound 1, Form VI has an X-ray powder diffraction (XRPD) pattern with characteristic peaks at 4.14±0.2° 2θ, 5.90±0.2° 2θ, 9.57±0.2° 2θ, 11.49±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least three characteristic peaks selected from 4.14±0.2° 2θ, 5.90±0.2° 2θ, 9.57±0.2° 2θ, 11.49±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least four characteristic peaks selected from 4.14±0.2° 2θ, 5.90±0.2° 2θ, 9.57±0.2° 2θ, 11.49±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least five characteristic peaks selected from 4.14±0.2° 2θ, 5.90±0.2° 2θ, 9.57±0.2° 2θ, 11.49±0.2° 2θ, 17.88±0.2° 2θ, 20.20±0.2° 2θ, and 20.88±0.2° 2θ.

In some embodiments, the X-ray powder diffraction pattern further comprises at least one peak selected from 4.43±0.2° 2θ, 7.98±0.2° 2θ, 11.90±0.2° 2θ, 14.54±0.2° 2θ, and 15.35±0.2° 2θ.

In some embodiments, the X-ray powder diffraction pattern further comprises at least two peaks selected from 4.43±0.2° 2θ, 7.98±0.2° 2θ, 11.90±0.2° 2θ, 14.54±0.2° 2θ, and 15.35±0.2° 2θ.

In some embodiments, the X-ray powder diffraction pattern further comprises at least three peaks selected from 4.43±0.2° 2θ, 7.98±0.2° 2θ, 11.90±0.2° 2θ, 14.54±0.2° 2θ, and 15.35±0.2° 2θ.

In some embodiments, the X-ray powder diffraction pattern further comprises peaks at 4.43±0.2° 2θ, 7.98±0.2° 2θ, 11.90±0.2° 2θ, 14.54±0.2° 2θ, and 15.35±0.2° 2θ.

In some embodiments, crystalline compound 1, Form VI has an X-ray powder diffraction (XRPD) pattern with characteristic peaks at 4.14±0.2° 2θ, 4.43±0.2° 2θ, 5.90±0.2° 2θ, 7.98±0.2° 2θ, 9.57±0.2° 2θ, 11.49±0.2° 2θ, 11.90±0.2° 2θ, 14.54±0.2° 2θ, 15.35±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least five characteristic peaks selected from 4.14±0.2° 2θ, 4.43±0.2° 2θ, 5.90±0.2° 2θ, 7.98±0.2° 2θ, 9.57±0.2° 2θ, 11.49±0.2° 2θ, 11.90±0.2° 2θ, 14.54±0.2° 2θ, 15.35±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least six characteristic peaks selected from 4.14±0.2° 2θ, 4.43±0.2° 2θ, 5.90±0.2° 2θ, 7.98±0.2° 2θ, 9.57±0.2° 2θ, 11.49±0.2° 2θ, 11.90±0.2° 2θ, 14.54±0.2° 2θ, 15.35±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least seven characteristic peaks selected from 4.14±0.2° 2θ, 4.43±0.2° 2θ, 5.90±0.2° 2θ, 7.98±0.2° 2θ, 9.57±0.2° 2θ, 11.49±0.2° 2θ, 11.90±0.2° 2θ, 14.54±0.2° 2θ, 15.35±0.2° 2θ, 17.88±0.2° 2θ, 20.20±0.2° 2θ, and 20.88±0.2° 2θ.

In some embodiments, crystalline compound 1, Form VI has an X-ray powder diffraction (XRPD) pattern with at least eight characteristic peaks selected from 4.14±0.2° 2θ, 4.43±0.2° 2θ, 5.90±0.2° 2θ, 7.98±0.2° 2θ, 9.57±0.2° 2θ, 11.49±0.2° 2θ, 11.90±0.2° 2θ, 14.54±0.2° 2θ, 15.35±0.2° 2θ, 17.88±0.2° 2θ, 20.20±0.2° 2θ, and 20.88±0.2° 2θ.

In some embodiments, the X-ray powder diffraction pattern further comprises at least one peak selected from 13.40±0.2° 2θ, 13.77±0.2° 2θ, and 19.53±0.2° 2θ.

In some embodiments, crystalline compound 1, Form VI has a DSC thermogram with an endotherm having an onset temperature at about 97° C. and a peak temperature at about 108° C.

In some embodiments, crystalline compound 1, Form VI has a thermogravimetric analysis (TGA) thermogram comprising a loss in mass of about 1% over a temperature range of about 70° C. to about 120° C.

In some embodiments, crystalline compound 1, Form VI is a solvate. In some embodiments, crystalline compound 1, Form VI is a MIBK solvate.

In some embodiments, crystalline compound 1, Form VI is an EtOAc and heptane solvate. In some embodiments, crystalline compound 1, Form VI is an EtOAc solvate. In some embodiments, crystalline compound 1, Form VI is a heptane solvate.

TABLE 5
Form VI

| 2-Theta | d(A) | BG | Height | I % | Area | I % | FWHM |
|---|---|---|---|---|---|---|---|
| 3.223 | 27.388 | 286 | 83 | 3 | 660 | 1.3 | 0.089 |
| 4.143 | 21.3074 | 267 | 835 | 29.8 | 15240 | 28.9 | 0.204 |
| 4.432 | 19.9205 | 267 | 343 | 12.2 | 5850 | 11.1 | 0.19 |
| 5.902 | 14.9633 | 267 | 2802 | 100 | 52728 | 100 | 0.21 |
| 6.399 | 13.8017 | 281 | 156 | 5.6 | 2960 | 5.6 | 0.212 |
| 7.988 | 11.0586 | 245 | 375 | 13.4 | 7145 | 13.6 | 0.213 |
| 8.306 | 10.6361 | 233 | 238 | 8.5 | 7224 | 13.7 | 0.339 |
| 9.566 | 9.2378 | 214 | 593 | 21.2 | 11580 | 22 | 0.218 |
| 11.495 | 7.6918 | 221 | 746 | 26.6 | 13785 | 26.1 | 0.206 |
| 11.904 | 7.4282 | 183 | 412 | 14.7 | 15999 | 30.3 | 0.433 |
| 12.518 | 7.0653 | 203 | 93 | 3.3 | 1372 | 2.6 | 0.165 |
| 13.399 | 6.6028 | 181 | 321 | 11.5 | 6272 | 11.9 | 0.218 |
| 13.766 | 6.4276 | 184 | 327 | 11.7 | 6191 | 11.7 | 0.211 |
| 14.541 | 6.0867 | 182 | 468 | 16.7 | 8709 | 16.5 | 0.208 |
| 15.355 | 5.7658 | 168 | 394 | 14.1 | 8352 | 15.8 | 0.237 |
| 15.618 | 5.6692 | 160 | 212 | 7.6 | 9445 | 17.9 | 0.497 |
| 16.014 | 5.5299 | 153 | 186 | 6.6 | 3869 | 7.3 | 0.232 |
| 16.668 | 5.3142 | 159 | 151 | 5.4 | 2403 | 4.6 | 0.178 |
| 17.155 | 5.1646 | 154 | 211 | 7.5 | 4868 | 9.2 | 0.257 |
| 17.877 | 4.9577 | 150 | 516 | 18.4 | 12807 | 24.3 | 0.277 |
| 18.495 | 4.7933 | 138 | 172 | 6.1 | 2852 | 5.4 | 0.185 |
| 19.531 | 4.5413 | 156 | 281 | 10 | 5146 | 9.8 | 0.204 |
| 20.201 | 4.3922 | 164 | 560 | 20 | 10792 | 20.5 | 0.215 |
| 20.884 | 4.2501 | 167 | 584 | 20.8 | 12027 | 22.8 | 0.23 |
| 21.593 | 4.1121 | 155 | 206 | 7.4 | 3965 | 7.5 | 0.215 |
| 22.052 | 4.0275 | 85 | 155 | 5.5 | 5879 | 11.1 | 0.423 |
| 23.111 | 3.8453 | 87 | 52 | 1.9 | 641 | 1.2 | 0.138 |
| 24.075 | 3.6935 | 94 | 131 | 4.7 | 3130 | 5.9 | 0.267 |
| 24.888 | 3.5746 | 97 | 189 | 6.7 | 3204 | 6.1 | 0.189 |
| 27.391 | 3.2534 | 81 | 47 | 1.7 | 1504 | 2.9 | 0.357 |
| 28.157 | 3.1666 | 91 | 108 | 3.9 | 1423 | 2.7 | 0.147 |
| 28.996 | 3.0768 | 79 | 58 | 2.1 | 2002 | 3.8 | 0.385 |
| 30.599 | 2.9192 | 66 | 71 | 2.5 | 1295 | 2.5 | 0.204 |
| 31.058 | 2.8771 | 70 | 60 | 2.1 | 640 | 1.2 | 0.119 |
| 32.505 | 2.7523 | 69 | 31 | 1.1 | 654 | 1.2 | 0.235 |
| 34.973 | 2.5635 | 50 | 40 | 1.4 | 757 | 1.4 | 0.211 |

### Polymorph Form VII of Compound 1

In some embodiments, compound 1 is crystalline. In some embodiments, crystalline compound 1 is Form VII characterized as having at least one of the following properties:
(a) an X-ray powder diffraction (XRPD) pattern with characteristic peaks at 5.22±0.2° 2θ, 7.45±0.2° 2θ, 10.43±0.2° 2θ, 14.39±0.2° 2θ, 17.13±0.2° 2θ, and 19.16±0.2° 2θ.
(b) a DSC thermogram with an endotherm having an onset temperature at about 85.5° C. and a peak temperature at about 98.5° C.;
(c) a DSC thermogram with an endotherm having an onset temperature at about 148° C. and a peak temperature at about 154° C.;
(d) a thermogravimetric analysis (TGA) thermogram comprising a loss in mass of about 8.9% over a temperature range of about 75° C. to about 120° C.;
(e) a thermogravimetric analysis (TGA) thermogram comprising a loss in mass of about 2.9% over a temperature range of about 123° C. to about 165° C.; or
(f) combinations thereof.

In some embodiments, crystalline compound 1, Form VII is characterized as having at least one of the properties selected from (a) to (e). In some embodiments, crystalline compound 1, Form VII is characterized as having at least two of the properties selected from (a) to (e). In some embodiments, crystalline compound 1, Form VII is characterized as having at least three of the properties selected from (a) to (e). In some embodiments, crystalline compound 1, Form VII is characterized as having at least four of the properties selected from (a) to (e). In some embodiments, crystalline compound 1, Form VII is characterized as having properties (a) to (e).

In some embodiments, crystalline compound 1, Form VII has an X-ray powder diffraction (XRPD) pattern with characteristic peaks found in Table 6. In some embodiments, crystalline compound 1, Form VII has an X-ray powder diffraction (XRPD) pattern with characteristic peaks at 5.22±0.2° 2θ, 7.45±0.2° 2θ, 10.43±0.2° 2θ, 14.39±0.2° 2θ, 17.13±0.2° 2θ, and 19.16±0.2° 2θ.

In some embodiments, crystalline compound 1, Form VII has an X-ray powder diffraction (XRPD) pattern with at least three characteristic peaks selected from 5.22±0.2° 2θ, 7.45±0.2° 2θ, 10.43±0.2° 2θ, 14.39±0.2° 2θ, 17.13±0.2° 2θ, and 19.16±0.2° 2θ.

In some embodiments, crystalline compound 1, Form VII has an X-ray powder diffraction (XRPD) pattern with at least four characteristic peaks selected from 5.22±0.2° 2θ, 7.45±0.2° 2θ, 10.43±0.2° 2θ, 14.39±0.2° 2θ, 17.13±0.2° 2θ, and 19.16±0.2° 2θ.

In some embodiments, crystalline compound 1, Form VII has an X-ray powder diffraction (XRPD) pattern with at least five characteristic peaks selected from 5.22±0.2° 2θ, 7.45±0.2° 2θ, 10.43±0.2° 2θ, 14.39±0.2° 2θ, 17.13±0.2° 2θ, and 19.16±0.2° 2θ.

In some embodiments, the X-ray powder diffraction pattern further comprises at least one peak selected from 10.19±0.2° 2θ, 11.03±0.2° 2θ, 15.67±0.2° 2θ, 18.47±0.2° 2θ, and 22.47±0.2° 2θ.

In some embodiments, the X-ray powder diffraction pattern further comprises at least two peaks selected from 10.19±0.2° 2θ, 11.03±0.2° 2θ, 15.67±0.2° 2θ, 18.47±0.2° 2θ, and 22.47±0.2° 2θ.

In some embodiments, the X-ray powder diffraction pattern further comprises at least three peaks selected from 10.19±0.2° 2θ, 11.03±0.2° 2θ, 15.67±0.2° 2θ, 18.47±0.2° 2θ, and 22.47±0.2° 2θ.

In some embodiments, the X-ray powder diffraction pattern further comprises peaks at 10.19±0.2° 2θ, 11.03±0.2° 2θ, 15.67±0.2° 2θ, 18.47±0.2° 2θ, and 22.47±0.2° 2θ.

In some embodiments, crystalline compound 1, Form VII has an X-ray powder diffraction (XRPD) pattern with characteristic peaks at 5.22±0.2° 2θ, 7.45±0.2° 2θ, 10.19±0.2° 2θ, 10.43±0.2° 2θ, 11.03±0.2° 2θ, 14.39±0.2° 2θ, 15.67±0.2° 2θ, 17.13±0.2° 2θ, 18.47±0.2° 2θ, 19.16±0.2° 2θ, and 22.47±0.2° 2θ.

In some embodiments, crystalline compound 1, Form VII has an X-ray powder diffraction (XRPD) pattern with at least five characteristic peaks selected from 5.22±0.2° 2θ, 7.45±0.2° 2θ, 10.19±0.2° 2θ, 10.43±0.2° 2θ, 11.03±0.2° 2θ, 14.39±0.2° 2θ, 15.67±0.2° 2θ, 17.13±0.2° 2θ, 18.47±0.2° 2θ, 19.16±0.2° 2θ, and 22.47±0.2° 2θ.

In some embodiments, crystalline compound 1, Form VII has an X-ray powder diffraction (XRPD) pattern with at least six characteristic peaks selected from 5.22±0.2° 2θ, 7.45±0.2° 2θ, 10.19±0.2° 2θ, 10.43±0.2° 2θ, 11.03±0.2° 2θ, 14.39±0.2° 2θ, 15.67±0.2° 2θ, 17.13±0.2° 2θ, 18.47±0.2° 2θ, 19.16±0.2° 2θ, and 22.47±0.2° 2θ.

In some embodiments, crystalline compound 1, Form VII has an X-ray powder diffraction (XRPD) pattern with at least seven characteristic peaks selected from 5.22±0.2° 2θ, 7.45±0.2° 2θ, 10.19±0.2° 2θ, 10.43±0.2° 2θ, 11.03±0.2° 2θ, 14.39±0.2° 2θ, 15.67±0.2° 2θ, 17.13±0.2° 2θ, 18.47±0.2° 2θ, 19.16±0.2° 2θ, and 22.47±0.2° 2θ.

In some embodiments, crystalline compound 1, Form VII has an X-ray powder diffraction (XRPD) pattern with at least eight characteristic peaks selected from 5.22±0.2° 2θ, 7.45±0.2° 2θ, 10.19±0.2° 2θ, 10.43±0.2° 2θ, 11.03±0.2° 2θ, 14.39±0.2° 2θ, 15.67±0.2° 2θ, 17.13±0.2° 2θ, 18.47±0.2° 2θ, 19.16±0.2° 2θ, and 22.47±0.2° 2θ.

In some embodiments, the X-ray powder diffraction pattern further comprises at least one peak selected from 14.92±0.2° 2θ, 24.47±0.2° 2θ, and 26.27±0.2° 2θ.

In some embodiments, crystalline compound 1, Form VII has a DSC thermogram with an endotherm having an onset temperature at about 85.5° C. and a peak temperature at about 98.5° C.

In some embodiments, crystalline compound 1, Form VII has a DSC thermogram with an endotherm having an onset temperature at about 148° C. and a peak temperature at about 154° C.

In some embodiments, crystalline compound 1, Form VII has a thermogravimetric analysis (TGA) thermogram comprising a loss in mass of about 8.9% over a temperature range of about 75° C. to about 120° C.

In some embodiments, crystalline compound 1, Form VII has a thermogravimetric analysis (TGA) thermogram comprising a loss in mass of about 2.9% over a temperature range of about 123° C. to about 165° C.

In some embodiments, crystalline compound 1, Form VII is a solvate. In some embodiments, crystalline compound 1, Form VII is a MTBE solvate. In some embodiments, crystalline compound 1, Form VII is a MEK and MeCYH (methylcyclohexane) solvate. In some embodiments, crystalline compound 1, Form VII is a MEK solvate. In some embodiments, crystalline compound 1, Form VII is a MeCYH solvate.

TABLE 6
Form VII

| 2-Theta | d(Å) | BG | Height | I % | Area | I % | FWHM |
|---|---|---|---|---|---|---|---|
| 5.216 | 16.927 | 250 | 7689 | 65.2 | 54502 | 63.1 | 0.079 |
| 7.451 | 11.8554 | 212 | 11800 | 100 | 86332 | 100 | 0.082 |
| 7.83 | 11.2815 | 212 | 168 | 1.4 | 2279 | 2.6 | 0.151 |
| 10.198 | 8.6672 | 173 | 807 | 6.8 | 11953 | 13.8 | 0.165 |
| 10.433 | 8.4725 | 173 | 3427 | 29 | 25902 | 30 | 0.084 |
| 11.035 | 8.0115 | 174 | 471 | 4 | 3793 | 4.4 | 0.09 |
| 14.397 | 6.1471 | 127 | 1513 | 12.8 | 13202 | 15.3 | 0.097 |
| 14.925 | 5.9309 | 124 | 610 | 5.2 | 5137 | 6 | 0.094 |
| 15.398 | 5.7497 | 119 | 88 | 0.7 | 846 | 1 | 0.107 |
| 15.672 | 5.6497 | 115 | 480 | 4.1 | 3981 | 4.6 | 0.093 |
| 17.13 | 5.172 | 100 | 3126 | 26.5 | 27583 | 31.9 | 0.098 |
| 18.466 | 4.8007 | 103 | 839 | 7.1 | 7179 | 8.3 | 0.095 |
| 19.163 | 4.6278 | 88 | 1318 | 11.2 | 12860 | 14.9 | 0.109 |
| 20.491 | 4.3306 | 84 | 391 | 3.3 | 3479 | 4 | 0.099 |
| 20.959 | 4.2349 | 81 | 356 | 3 | 3848 | 4.5 | 0.121 |
| 21.287 | 4.1706 | 84 | 37 | 0.3 | 568 | 0.7 | 0.171 |
| 21.641 | 4.1031 | 83 | 73 | 0.6 | 471 | 0.5 | 0.072 |
| 22.159 | 4.0083 | 76 | 203 | 1.7 | 2177 | 2.5 | 0.12 |
| 22.472 | 3.9532 | 71 | 853 | 7.2 | 7839 | 9.1 | 0.103 |
| 23.64 | 3.7604 | 63 | 114 | 1 | 944 | 1.1 | 0.092 |
| 24.469 | 3.6348 | 61 | 473 | 4 | 6047 | 7 | 0.143 |
| 26.268 | 3.3899 | 51 | 416 | 3.5 | 4114 | 4.8 | 0.11 |
| 27.37 | 3.2558 | 44 | 52 | 0.4 | 893 | 1 | 0.192 |
| 29.088 | 3.0673 | 43 | 63 | 0.5 | 1189 | 1.4 | 0.211 |
| 29.302 | 3.0455 | 44 | 248 | 2.1 | 2831 | 3.3 | 0.127 |
| 29.591 | 3.0164 | 41 | 98 | 0.8 | 1426 | 1.7 | 0.162 |
| 30.941 | 2.8877 | 35 | 55 | 0.5 | 550 | 0.6 | 0.112 |
| 31.651 | 2.8246 | 35 | 224 | 1.9 | 2518 | 2.9 | 0.125 |
| 32.006 | 2.7941 | 36 | 25 | 0.2 | 291 | 0.3 | 0.13 |
| 33.845 | 2.6463 | 32 | 32 | 0.3 | 560 | 0.6 | 0.195 |
| 35.71 | 2.5123 | 30 | 72 | 0.6 | 940 | 1.1 | 0.146 |
| 37.928 | 2.3703 | 33 | 97 | 0.8 | 915 | 1.1 | 0.105 |
| 38.883 | 2.3142 | 27 | 132 | 1.1 | 2109 | 2.4 | 0.178 |
| 39.474 | 2.2809 | 27 | 40 | 0.3 | 690 | 0.8 | 0.193 |

### Polymorph Form VIII of Compound 1

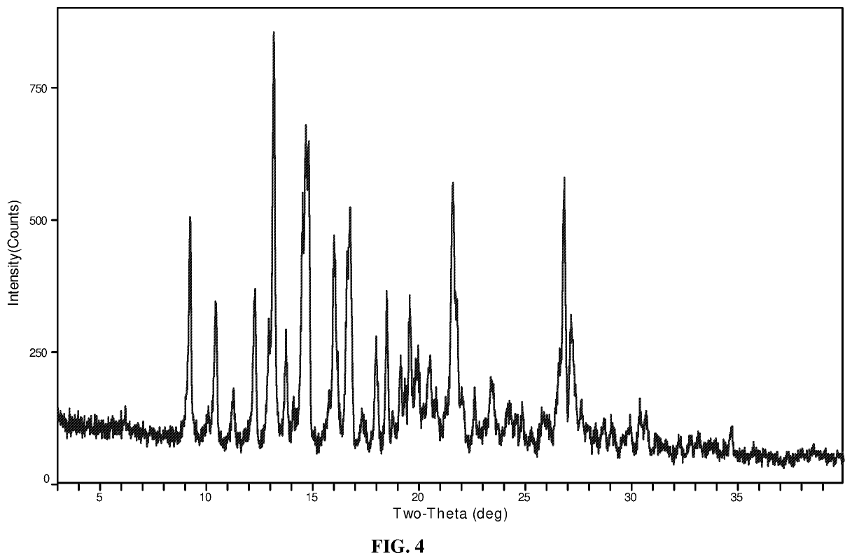

In some embodiments, compound 1 is crystalline. In some embodiments, crystalline compound 1 is Form VIII characterized as having at least one of the following properties:
(a) an X-ray powder diffraction (XRPD) pattern substantially the same as shown in FIG. 4;
(b) an X-ray powder diffraction (XRPD) pattern with characteristic peaks at 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.
(c) a DSC thermogram with an endotherm having an onset temperature at about 48° C. and a peak temperature at about 49° C.;
(d) a DSC thermogram with an endotherm having an onset temperature at about 154° C. and a peak temperature at about 155° C.;
(e) a thermogravimetric analysis (TGA) thermogram comprising a loss in mass of about 0.23% over a temperature range of about 30° C. to about 50° C.; or
(f) combinations thereof.

In some embodiments, crystalline compound 1, Form VIII is characterized as having at least one of the properties selected from (a) to (e). In some embodiments, crystalline compound 1, Form VIII is characterized as having at least two of the properties selected from (a) to (e). In some embodiments, crystalline compound 1, Form VIII is characterized as having at least three of the properties selected from (a) to (e). In some embodiments, crystalline compound 1, Form VIII is characterized as having at least four of the properties selected from (a) to (e). In some embodiments, crystalline compound 1, Form VIII is characterized as having properties (a) to (e).

In some embodiments, crystalline compound 1, Form VIII has an X-ray powder diffraction (XRPD) pattern substantially the same as shown in FIG. 4. In some embodiments, crystalline compound 1, Form VIII has an X-ray powder diffraction (XRPD) pattern with characteristic peaks found in Table 7. In some embodiments, crystalline compound 1, Form VIII has an X-ray powder diffraction (XRPD) pattern with characteristic peaks at 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.

In some embodiments, crystalline compound 1, Form VIII has an X-ray powder diffraction (XRPD) pattern with at least three characteristic peaks selected from 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.

In some embodiments, crystalline compound 1, Form VIII has an X-ray powder diffraction (XRPD) pattern with at least four characteristic peaks selected from 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.

In some embodiments, crystalline compound 1, Form VIII has an X-ray powder diffraction (XRPD) pattern with at least five characteristic peaks selected from 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.

In some embodiments, the X-ray powder diffraction pattern further comprises at least one peak selected from 10.43±0.2° 2θ, 12.94±0.2° 2θ, 17.99±0.2° 2θ, 19.57±0.2° 2θ, 21.80±0.2° 2θ, and 27.16±0.2° 2θ.

In some embodiments, the X-ray powder diffraction pattern further comprises at least two peaks selected from 10.43±0.2° 2θ, 12.94±0.2° 2θ, 17.99±0.2° 2θ, 19.57±0.2° 2θ, 21.80±0.2° 2θ, and 27.16±0.2° 2θ.

In some embodiments, the X-ray powder diffraction pattern further comprises at least three peaks selected from 10.43±0.2° 2θ, 12.94±0.2° 2θ, 17.99±0.2° 2θ, 19.57±0.2° 2θ, 21.80±0.2° 2θ, and 27.16±0.2° 2θ.

In some embodiments, the X-ray powder diffraction pattern further comprises peaks at 10.43±0.2° 2θ, 12.94±0.2° 2θ, 17.99±0.2° 2θ, 19.57±0.2° 2θ, 21.80±0.2° 2θ, and 27.16±0.2° 2θ.

In some embodiments, crystalline compound 1, Form VIII has an X-ray powder diffraction (XRPD) pattern with characteristic peaks at 9.23±0.2° 2θ, 10.43±0.2° 2θ, 12.29±0.2° 2θ, 12.94±0.2° 2θ, 13.18±0.2° 2θ, 14.83±0.2° 2θ, 16.01±0.2° 2θ, 16.76±0.2° 2θ, 17.99±0.2° 2θ, 18.48±0.2° 2θ, 19.57±0.2° 2θ, 21.80±0.2° 2θ, 26.84±0.2° 2θ, and 27.16±0.2° 2θ.

In some embodiments, crystalline compound 1, Form VIII has an X-ray powder diffraction (XRPD) pattern with at least five characteristic peaks selected from 9.23±0.2° 2θ, 10.43±0.2° 2θ, 12.29±0.2° 2θ, 12.94±0.2° 2θ, 13.18±0.2° 2θ, 14.83±0.2° 2θ, 16.01±0.2° 2θ, 16.76±0.2° 2θ, 17.99±0.2° 2θ, 18.48±0.2° 2θ, 19.57±0.2° 2θ, 21.80±0.2° 2θ, 26.84±0.2° 2θ, and 27.16±0.2° 2θ.

In some embodiments, crystalline compound 1, Form VIII has an X-ray powder diffraction (XRPD) pattern with at least six characteristic peaks selected from 9.23±0.2° 2θ, 10.43±0.2° 2θ, 12.29±0.2° 2θ, 12.94±0.2° 2θ, 13.18±0.2° 2θ, 14.83±0.2° 2θ, 16.01±0.2° 2θ, 16.76±0.2° 2θ, 17.99±0.2° 2θ, 18.48±0.2° 2θ, 19.57±0.2° 2θ, 21.80±0.2° 2θ, 26.84±0.2° 2θ, and 27.16±0.2° 2θ.

In some embodiments, crystalline compound 1, Form VIII has an X-ray powder diffraction (XRPD) pattern with at least seven characteristic peaks selected from 9.23±0.2° 2θ, 10.43±0.2° 2θ, 12.29±0.2° 2θ, 12.94±0.2° 2θ, 13.18±0.2° 2θ, 14.83±0.2° 2θ, 16.01±0.2° 2θ, 16.76±0.2° 2θ, 17.99±0.2° 2θ, 18.48±0.2° 2θ, 19.57±0.2° 2θ, 21.80±0.2° 2θ, 26.84±0.2° 2θ, and 27.16±0.2° 2θ.

In some embodiments, crystalline compound 1, Form VIII has an X-ray powder diffraction (XRPD) pattern with at least eight characteristic peaks selected from 9.23±0.2° 2θ, 10.43±0.2° 2θ, 12.29±0.2° 2θ, 12.94±0.2° 2θ, 13.18±0.2° 2θ, 14.83±0.2° 2θ, 16.01±0.2° 2θ, 16.76±0.2° 2θ, 17.99±0.2° 2θ, 18.48±0.2° 2θ, 19.57±0.2° 2θ, 21.80±0.2° 2θ, 26.84±0.2° 2θ, and 27.16±0.2° 2θ.

In some embodiments, the X-ray powder diffraction pattern further comprises at least one peak selected from 13.74±0.2° 2θ, 19.14±0.2° 2θ, and 19.96±0.2° 2θ.

In some embodiments, crystalline compound 1, Form VIII has a DSC thermogram with an endotherm having an onset temperature at about 48° C. and a peak temperature at about 49° C.

In some embodiments, crystalline compound 1, Form VIII has a DSC thermogram with an endotherm having an onset temperature at about 154° C. and a peak temperature at about 155° C.

In some embodiments, crystalline compound 1, Form VIII has a DSC thermogram with an endotherm having an onset temperature at about 48° C. and a peak temperature at about 49° C. and an onset temperature at about 154° C. and a peak temperature at about 155° C.

In some embodiments, crystalline compound 1, Form VIII has a thermogravimetric analysis (TGA) thermogram comprising a loss in mass of about 0.23% over a temperature range of about 30° C. to about 50° C.

In some embodiments, crystalline compound 1, Form VIII is anhydrous.

TABLE 7
Form VIII

| 2-Theta | d(Å) | BG | Height | I % | Area | I % | FWHM |
|---|---|---|---|---|---|---|---|
| 6.193 | 14.2601 | 95 | 51 | 6.6 | 1081 | 6.4 | 0.237 |
| 9.237 | 9.5663 | 84 | 422 | 54.6 | 5946 | 35.4 | 0.157 |
| 10.102 | 8.7493 | 79 | 67 | 8.7 | 1321 | 7.9 | 0.22 |
| 10.433 | 8.4723 | 78 | 268 | 34.7 | 4623 | 27.5 | 0.193 |
| 11.284 | 7.8348 | 75 | 105 | 13.6 | 1493 | 8.9 | 0.159 |
| 12.295 | 7.1928 | 73 | 296 | 38.3 | 4469 | 26.6 | 0.169 |
| 12.94 | 6.836 | 79 | 234 | 30.3 | 9052 | 53.9 | 0.432 |
| 13.176 | 6.7139 | 85 | 773 | 100 | 12643 | 75.2 | 0.183 |
| 13.742 | 6.4387 | 102 | 190 | 24.6 | 1738 | 10.3 | 0.102 |
| 14.109 | 6.272 | 99 | 65 | 8.4 | 707 | 4.2 | 0.121 |
| 14.828 | 5.9694 | 80 | 570 | 73.7 | 16806 | 100 | 0.329 |
| 15.765 | 5.6167 | 86 | 88 | 11.4 | 2013 | 12 | 0.255 |
| 16.013 | 5.5304 | 73 | 398 | 51.5 | 8968 | 53.4 | 0.251 |
| 16.761 | 5.2851 | 92 | 432 | 55.9 | 8529 | 50.7 | 0.22 |
| 17.313 | 5.1177 | 74 | 68 | 8.8 | 1043 | 6.2 | 0.171 |
| 17.994 | 4.9256 | 72 | 207 | 26.8 | 2606 | 15.5 | 0.141 |
| 18.483 | 4.7963 | 81 | 284 | 36.7 | 2793 | 16.6 | 0.11 |
| 18.767 | 4.7243 | 92 | 45 | 5.8 | 244 | 1.5 | 0.061 |
| 19.137 | 4.634 | 101 | 142 | 18.4 | 2177 | 13 | 0.171 |
| 19.571 | 4.5321 | 114 | 243 | 31.4 | 3562 | 21.2 | 0.164 |
| 19.964 | 4.4439 | 103 | 159 | 20.6 | 4044 | 24.1 | 0.284 |
| 20.529 | 4.3228 | 119 | 124 | 16 | 2555 | 15.2 | 0.23 |
| 20.816 | 4.2638 | 111 | 69 | 8.9 | 916 | 5.5 | 0.148 |
| 21.253 | 4.177 | 103 | 61 | 7.9 | 878 | 5.2 | 0.161 |
| 21.605 | 4.1098 | 92 | 479 | 62 | 12137 | 72.2 | 0.283 |
| 21.801 | 4.0733 | 88 | 262 | 33.9 | 10319 | 61.4 | 0.44 |
| 22.066 | 4.025 | 81 | 83 | 10.7 | 3137 | 18.7 | 0.422 |
| 22.617 | 3.9281 | 82 | 100 | 12.9 | 920 | 5.5 | 0.103 |
| 23.104 | 3.8465 | 79 | 44 | 5.7 | 1675 | 10 | 0.425 |
| 23.379 | 3.8019 | 81 | 121 | 15.7 | 3446 | 20.5 | 0.318 |
| 24.261 | 3.6656 | 81 | 75 | 9.7 | 2322 | 13.8 | 0.346 |
| 24.573 | 3.6198 | 79 | 51 | 6.6 | 1166 | 6.9 | 0.255 |
| 25.875 | 3.4405 | 81 | 54 | 7 | 1288 | 7.7 | 0.266 |
| 26.141 | 3.4061 | 87 | 43 | 5.6 | 1008 | 6 | 0.262 |
| 26.57 | 3.352 | 94 | 134 | 17.3 | 6055 | 36 | 0.504 |
| 26.843 | 3.3185 | 93 | 488 | 63.1 | 10439 | 62.1 | 0.239 |
| 27.16 | 3.2805 | 84 | 236 | 30.5 | 8164 | 48.6 | 0.386 |
| 27.659 | 3.2224 | 103 | 56 | 7.2 | 652 | 3.9 | 0.13 |
| 28.32 | 3.1488 | 74 | 40 | 5.2 | 246 | 1.5 | 0.069 |
| 28.707 | 3.1072 | 69 | 54 | 7 | 1056 | 6.3 | 0.218 |
| 29.098 | 3.0663 | 64 | 50 | 6.5 | 945 | 5.6 | 0.211 |
| 29.588 | 3.0166 | 62 | 30 | 3.9 | 826 | 4.9 | 0.307 |
| 29.93 | 2.9829 | 61 | 69 | 8.9 | 1461 | 8.7 | 0.236 |
| 30.389 | 2.939 | 67 | 94 | 12.2 | 1760 | 10.5 | 0.209 |
| 30.678 | 2.9119 | 70 | 67 | 8.7 | 1237 | 7.4 | 0.206 |
| 31.164 | 2.8675 | 58 | 34 | 4.4 | 959 | 5.7 | 0.315 |
| 32.318 | 2.7677 | 55 | 33 | 4.3 | 371 | 2.2 | 0.125 |
| 32.751 | 2.7321 | 57 | 33 | 4.3 | 654 | 3.9 | 0.221 |
| 33.213 | 2.6952 | 55 | 39 | 5 | 1627 | 9.7 | 0.466 |
| 34.732 | 2.5807 | 51 | 57 | 7.4 | 1017 | 6.1 | 0.199 |

### Polymorph Form IX of Compound 1

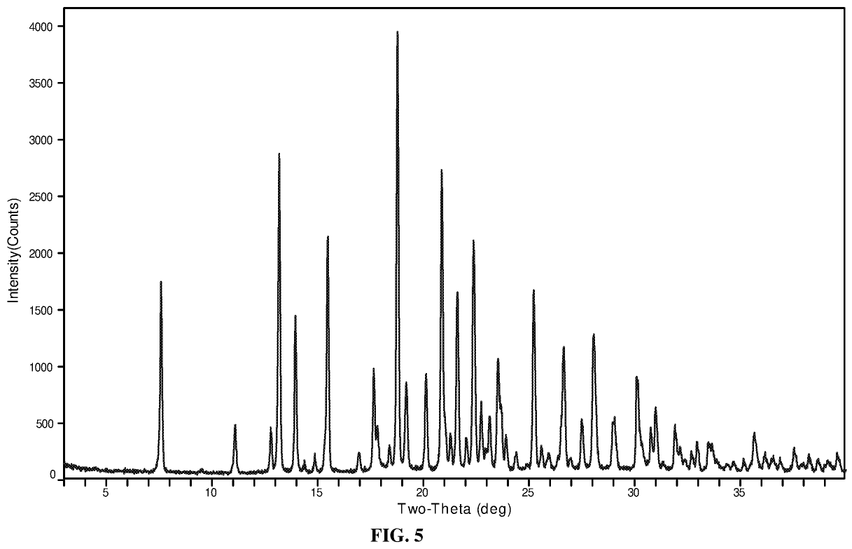

In some embodiments, compound 1 is crystalline. In some embodiments, crystalline compound 1 is Form IX characterized as having at least one of the following properties:
(a) an X-ray powder diffraction (XRPD) pattern substantially the same as shown in FIG. 5;
(b) an X-ray powder diffraction (XRPD) pattern with characteristic peaks at 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.
(c) a DSC thermogram with an endotherm having an onset temperature at about 29° C. and a peak temperature at about 55.5° C.;
(d) a DSC thermogram with an endotherm having an onset temperature at about 113.5° C. and a peak temperature at about 118° C.;
(e) a thermogravimetric analysis (TGA) thermogram comprising a loss in mass of about 1.39% over a temperature range of about 27° C. to about 80° C.; or
(f) combinations thereof.

In some embodiments, crystalline compound 1, Form IX is characterized as having at least one of the properties selected from (a) to (e). In some embodiments, crystalline compound 1, Form IX is characterized as having at least two of the properties selected from (a) to (e). In some embodiments, crystalline compound 1, Form IX is characterized as having at least three of the properties selected from (a) to (e). In some embodiments, crystalline compound 1, Form IX is characterized as having at least four of the properties selected from (a) to (e). In some embodiments, crystalline compound 1, Form IX is characterized as having properties (a) to (e).

In some embodiments, crystalline compound 1, Form IX has an X-ray powder diffraction (XRPD) pattern substantially the same as shown in FIG. 5. In some embodiments, crystalline compound 1, Form IX has an X-ray powder diffraction (XRPD) pattern with characteristic peaks found in Table 8. In some embodiments, crystalline compound 1, Form IX has an X-ray powder diffraction (XRPD) pattern with characteristic peaks at 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.

In some embodiments, crystalline compound 1, Form IX has an X-ray powder diffraction (XRPD) pattern with at least three characteristic peaks selected from 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.

In some embodiments, crystalline compound 1, Form IX has an X-ray powder diffraction (XRPD) pattern with at least four characteristic peaks selected from 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.

In some embodiments, crystalline compound 1, Form IX has an X-ray powder diffraction (XRPD) pattern with at least five characteristic peaks selected from 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.

In some embodiments, the X-ray powder diffraction pattern further comprises at least one peak selected from 17.65±0.2° 2θ, 19.19±0.2° 2θ, 20.14±0.2° 2θ, 23.54±0.2° 2θ, 26.65±0.2° 2θ, and 30.1±0.2° 2θ.

In some embodiments, the X-ray powder diffraction pattern further comprises at least two peaks selected from 17.65±0.2° 2θ, 19.19±0.2° 2θ, 20.14±0.2° 2θ, 23.54±0.2° 2θ, 26.65±0.2° 2θ, and 30.1±0.2° 2θ.

In some embodiments, the X-ray powder diffraction pattern further comprises at least three peaks selected from 17.65±0.2° 2θ, 19.19±0.2° 2θ, 20.14±0.2° 2θ, 23.54±0.2° 2θ, 26.65±0.2° 2θ, and 30.1±0.2° 2θ.

In some embodiments, the X-ray powder diffraction pattern further comprises peaks at 17.65±0.2° 2θ, 19.19±0.2° 2θ, 20.14±0.2° 2θ, 23.54±0.2° 2θ, 26.65±0.2° 2θ, and 30.1±0.2° 2θ.

In some embodiments, crystalline compound 1, Form IX has an X-ray powder diffraction (XRPD) pattern with characteristic peaks at 7.59±0.2° 2θ, 13.18±0.2° 2θ, 13.95±0.2° 2θ, 15.48±0.2° 2θ, 17.65±0.2° 2θ, 18.78±0.2° 2θ, 19.19±0.2° 2θ, 20.14±0.2° 2θ, 20.87±0.2° 2θ, 21.62±0.2° 2θ, 23.37±0.2° 2θ, 23.54±0.2° 2θ, 26.65±0.2° 2θ, 28.07±0.2° 2θ, and 30.1±0.2° 2θ.

In some embodiments, crystalline compound 1, Form IX has an X-ray powder diffraction (XRPD) pattern with at least five characteristic peaks selected from 7.59±0.2° 2θ, 13.18±0.2° 2θ, 13.95±0.2° 2θ, 15.48±0.2° 2θ, 17.65±0.2° 2θ, 18.78±0.2° 2θ, 19.19±0.2° 2θ, 20.14±0.2° 2θ, 20.87±0.2° 2θ, 21.62±0.2° 2θ, 23.37±0.2° 2θ, 23.54±0.2° 2θ, 26.65±0.2° 2θ, 28.07±0.2° 2θ, and 30.1±0.2° 2θ.

In some embodiments, crystalline compound 1, Form IX has an X-ray powder diffraction (XRPD) pattern with at least six characteristic peaks selected from 7.59±0.2° 2θ, 13.18±0.2° 2θ, 13.95±0.2° 2θ, 15.48±0.2° 2θ, 17.65±0.2° 2θ, 18.78±0.2° 2θ, 19.19±0.2° 2θ, 20.14±0.2° 2θ, 20.87±0.2° 2θ, 21.62±0.2° 2θ, 23.37±0.2° 2θ, 23.54±0.2° 2θ, 26.65±0.2° 2θ, 28.07±0.2° 2θ, and 30.1±0.2° 2θ.

In some embodiments, crystalline compound 1, Form IX has an X-ray powder diffraction (XRPD) pattern with at least seven characteristic peaks selected from 7.59±0.2° 2θ, 13.18±0.2° 2θ, 13.95±0.2° 2θ, 15.48±0.2° 2θ, 17.65±0.2° 2θ, 18.78±0.2° 2θ, 19.19±0.2° 2θ, 20.14±0.2° 2θ, 20.87±0.2° 2θ, 21.62±0.2° 2θ, 23.37±0.2° 2θ, 23.54±0.2° 2θ, 26.65±0.2° 2θ, 28.07±0.2° 2θ, and 30.1±0.2° 2θ.

In some embodiments, crystalline compound 1, Form IX has an X-ray powder diffraction (XRPD) pattern with at least eight characteristic peaks selected from 7.59±0.2° 2θ, 13.18±0.2° 2θ, 13.95±0.2° 2θ, 15.48±0.2° 2θ, 17.65±0.2° 2θ, 18.78±0.2° 2θ, 19.19±0.2° 2θ, 20.14±0.2° 2θ, 20.87±0.2° 2θ, 21.62±0.2° 2θ, 23.37±0.2° 2θ, 23.54±0.2° 2θ, 26.65±0.2° 2θ, 28.07±0.2° 2θ, and 30.1±0.2° 2θ.

In some embodiments, the X-ray powder diffraction pattern further comprises at least one peak selected from 11.09±0.2° 2θ, 27.49±0.2° 2θ, and 30.99±0.2° 2θ.

In some embodiments, crystalline compound 1, Form IX has a DSC thermogram with an endotherm having an onset temperature at about 29° C. and a peak temperature at about 55.5° C.

In some embodiments, crystalline compound 1, Form IX has a DSC thermogram with an endotherm having an onset temperature at about 113.5° C. and a peak temperature at about 118° C.

In some embodiments, crystalline compound 1, Form IX has a DSC thermogram with an endotherm having an onset temperature at about 29° C. and a peak temperature at about 55.5° C. and an onset temperature at about 113.5° C. and a peak temperature at about 118° C.

In some embodiments, crystalline compound 1, Form IX has a thermogravimetric analysis (TGA) thermogram comprising a loss in mass of about 1.39% over a temperature range of about 27° C. to about 80° C.

In some embodiments, crystalline compound 1, Form IX is a hydrate.

TABLE 8
Form IX

| 2-Theta | d(Å) | BG | Height | I % | Area | I % | FWHM |
|---|---|---|---|---|---|---|---|
| 7.597 | 11.627 | 57 | 1687 | 43.7 | 15735 | 43.7 | 0.109 |
| 11.097 | 7.9668 | 51 | 426 | 11 | 4298 | 11.9 | 0.118 |
| 12.785 | 6.9181 | 59 | 396 | 10.3 | 4065 | 11.3 | 0.12 |
| 13.182 | 6.7109 | 65 | 2815 | 73 | 25245 | 70.1 | 0.105 |
| 13.95 | 6.343 | 68 | 1374 | 35.6 | 12188 | 33.8 | 0.103 |
| 14.374 | 6.1571 | 64 | 98 | 2.5 | 761 | 2.1 | 0.091 |
| 14.87 | 5.9525 | 63 | 159 | 4.1 | 992 | 2.8 | 0.073 |
| 15.485 | 5.7174 | 62 | 2084 | 54 | 20992 | 58.3 | 0.117 |
| 16.956 | 5.2248 | 71 | 160 | 4.1 | 1568 | 4.4 | 0.114 |
| 17.656 | 5.019 | 91 | 884 | 22.9 | 8962 | 24.9 | 0.118 |
| 17.832 | 4.9699 | 97 | 370 | 9.6 | 9321 | 25.9 | 0.294 |
| 18.396 | 4.8188 | 93 | 204 | 5.3 | 3300 | 9.2 | 0.189 |
| 18.78 | 4.7211 | 104 | 3858 | 100 | 36020 | 100 | 0.109 |
| 19.192 | 4.6207 | 100 | 754 | 19.5 | 8315 | 23.1 | 0.129 |
| 20.137 | 4.406 | 92 | 836 | 21.7 | 7896 | 21.9 | 0.11 |
| 20.868 | 4.2534 | 99 | 2637 | 68.4 | 28411 | 78.9 | 0.126 |
| 21.278 | 4.1723 | 113 | 289 | 7.5 | 3041 | 8.4 | 0.123 |
| 21.619 | 4.1071 | 126 | 1525 | 39.5 | 15130 | 42 | 0.116 |
| 22.022 | 4.0329 | 138 | 224 | 5.8 | 1726 | 4.8 | 0.09 |
| 22.376 | 3.9699 | 130 | 1981 | 51.3 | 21159 | 58.7 | 0.125 |
| 22.745 | 3.9064 | 130 | 553 | 14.3 | 5368 | 14.9 | 0.113 |
| 23.143 | 3.8401 | 121 | 432 | 11.2 | 5969 | 16.6 | 0.161 |
| 23.541 | 3.776 | 119 | 942 | 24.4 | 14870 | 41.3 | 0.184 |
| 23.678 | 3.7545 | 113 | 545 | 14.1 | 12036 | 33.4 | 0.258 |
| 23.912 | 3.7182 | 101 | 286 | 7.4 | 2854 | 7.9 | 0.116 |
| 24.406 | 3.6442 | 89 | 146 | 3.8 | 1353 | 3.8 | 0.108 |
| 25.229 | 3.5271 | 88 | 1581 | 41 | 17521 | 48.6 | 0.129 |
| 25.586 | 3.4787 | 89 | 204 | 5.3 | 2544 | 7.1 | 0.145 |
| 25.917 | 3.435 | 96 | 131 | 3.4 | 1611 | 4.5 | 0.143 |
| 26.655 | 3.3415 | 93 | 1074 | 27.8 | 15767 | 43.8 | 0.171 |
| 26.983 | 3.3016 | 95 | 89 | 2.3 | 1050 | 2.9 | 0.138 |
| 27.494 | 3.2414 | 95 | 432 | 11.2 | 5029 | 14 | 0.136 |
| 28.069 | 3.1763 | 94 | 1186 | 30.7 | 17037 | 47.3 | 0.168 |
| 29.056 | 3.0706 | 92 | 454 | 11.8 | 7153 | 19.9 | 0.184 |
| 30.1 | 2.9664 | 110 | 792 | 20.5 | 11931 | 33.1 | 0.176 |
| 30.759 | 2.9044 | 90 | 363 | 9.4 | 6122 | 17 | 0.197 |
| 30.992 | 2.8831 | 108 | 524 | 13.6 | 7162 | 19.9 | 0.159 |
| 31.359 | 2.8502 | 104 | 50 | 1.3 | 205 | 0.6 | 0.048 |
| 31.911 | 2.8021 | 92 | 387 | 10 | 7022 | 19.5 | 0.212 |
| 32.145 | 2.7822 | 92 | 192 | 5 | 4939 | 13.7 | 0.3 |
| 32.377 | 2.7628 | 83 | 89 | 2.3 | 1880 | 5.2 | 0.246 |
| 32.682 | 2.7378 | 95 | 148 | 3.8 | 1570 | 4.4 | 0.124 |
| 32.945 | 2.7165 | 85 | 242 | 6.3 | 2643 | 7.3 | 0.127 |
| 33.488 | 2.6737 | 80 | 242 | 6.3 | 6186 | 17.2 | 0.298 |
| 33.654 | 2.6609 | 82 | 227 | 5.9 | 6175 | 17.1 | 0.317 |
| 34.697 | 2.5832 | 81 | 70 | 1.8 | 1316 | 3.7 | 0.219 |
| 35.153 | 2.5508 | 80 | 98 | 2.5 | 586 | 1.6 | 0.07 |
| 35.67 | 2.515 | 98 | 309 | 8 | 4308 | 12 | 0.163 |
| 36.176 | 2.4809 | 76 | 159 | 4.1 | 2563 | 7.1 | 0.188 |
| 36.564 | 2.4555 | 91 | 113 | 2.9 | 1687 | 4.7 | 0.174 |
| 36.88 | 2.4352 | 88 | 100 | 2.6 | 730 | 2 | 0.085 |
| 37.55 | 2.3933 | 76 | 199 | 5.2 | 2930 | 8.1 | 0.172 |
| 38.25 | 2.3511 | 76 | 141 | 3.7 | 2403 | 6.7 | 0.199 |
| 38.687 | 2.3255 | 78 | 97 | 2.5 | 899 | 2.5 | 0.108 |
| 39.143 | 2.2994 | 72 | 91 | 2.4 | 2020 | 5.6 | 0.259 |
| 39.582 | 2.275 | 73 | 158 | 4.1 | 2190 | 6.1 | 0.162 |

### Polymorph Form X of Compound 1

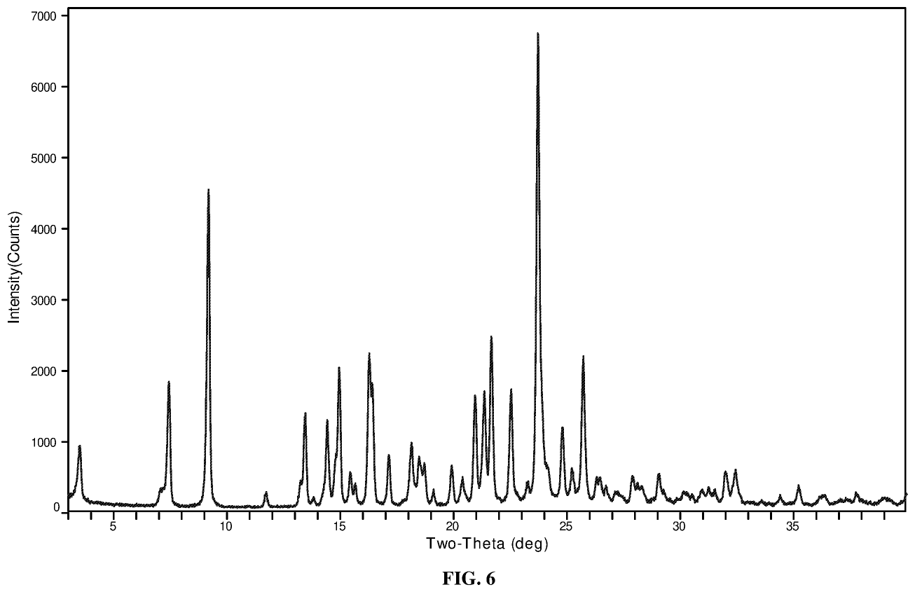

In some embodiments, compound 1 is crystalline. In some embodiments, crystalline compound 1 is Form X characterized as having at least one of the following properties:
(a) an X-ray powder diffraction (XRPD) pattern substantially the same as shown in FIG. 6;
(b) an X-ray powder diffraction (XRPD) pattern with characteristic peaks at 9.17±0.2° 2θ, 16.29±0.2° 2θ, 21.67±0.2° 2θ, and 23.72±0.2° 2θ.
(c) a DSC thermogram with an endotherm having an onset temperature at about 157° C. and a peak temperature at about 158° C.; or
(d) combinations thereof.

In some embodiments, the X-ray powder diffraction pattern further comprises peaks at 13.44±0.2° 2θ, 14.95±0.2° 2θ, and 25.72±0.2° 2θ.

In some embodiments, compound 1 is crystalline. In some embodiments, crystalline compound 1 is Form X characterized as having at least one of the following properties:
(a) an X-ray powder diffraction (XRPD) pattern substantially the same as shown in FIG. 6;
(b) an X-ray powder diffraction (XRPD) pattern with characteristic peaks at 9.17±0.2° 2θ, 13.44±0.2° 2θ, 14.95±0.2° 2θ, 16.29±0.2° 2θ, 21.67±0.2° 2θ, 23.72±0.2° 2θ, and 25.72±0.2° 2θ.
(c) a DSC thermogram with an endotherm having an onset temperature at about 157° C. and a peak temperature at about 158° C.; or
(d) combinations thereof.

In some embodiments, crystalline compound 1, Form X is characterized as having at least one of the properties selected from (a) to (c). In some embodiments, crystalline compound 1, Form X is characterized as having at least two of the properties selected from (a) to (c). In some embodiments, crystalline compound 1, Form X is characterized as having properties (a) to (c).

In some embodiments, crystalline compound 1, Form X has an X-ray powder diffraction (XRPD) pattern substantially the same as shown in FIG. 6. In some embodiments, crystalline compound 1, Form X has an X-ray powder diffraction (XRPD) pattern with characteristic peaks found in Table 9. In some embodiments, crystalline compound 1, Form X has an X-ray powder diffraction (XRPD) pattern with characteristic peaks at 9.17±0.2° 2θ, 13.44±0.2° 2θ, 14.95±0.2° 2θ, 16.29±0.2° 2θ, 21.67±0.2° 2θ, 23.72±0.2° 2θ, and 25.72±0.2° 2θ.

In some embodiments, crystalline compound 1, Form X has an X-ray powder diffraction (XRPD) pattern with at least three characteristic peaks selected from 9.17±0.2° 2θ, 13.44±0.2° 2θ, 14.95±0.2° 2θ, 16.29±0.2° 2θ, 21.67±0.2° 2θ, 23.72±0.2° 2θ, and 25.72±0.2° 2θ.

In some embodiments, crystalline compound 1, Form X has an X-ray powder diffraction (XRPD) pattern with at least four characteristic peaks selected from 9.17±0.2° 2θ, 13.44±0.2° 2θ, 14.95±0.2° 2θ, 16.29±0.2° 2θ, 21.67±0.2° 2θ, 23.72±0.2° 2θ, and 25.72±0.2° 2θ.

In some embodiments, crystalline compound 1, Form X has an X-ray powder diffraction (XRPD) pattern with at least five characteristic peaks selected from 9.17±0.2° 2θ, 13.44±0.2° 2θ, 14.95±0.2° 2θ, 16.29±0.2° 2θ, 21.67±0.2° 2θ, 23.72±0.2° 2θ, and 25.72±0.2° 2θ.

In some embodiments, the X-ray powder diffraction pattern further comprises at least one peak selected from 7.46±0.2° 2θ, 18.14±0.2° 2θ, 20.95±0.2° 2θ, 22.53±0.2° 2θ, and 24.80±0.2° 2θ.

In some embodiments, the X-ray powder diffraction pattern further comprises at least two peaks selected from 7.46±0.2° 2θ, 18.14±0.2° 2θ, 20.95±0.2° 2θ, 22.53±0.2° 2θ, and 24.80±0.2° 2θ.

In some embodiments, the X-ray powder diffraction pattern further comprises at least three peaks selected from 7.46±0.2° 2θ, 18.14±0.2° 2θ, 20.95±0.2° 2θ, 22.53±0.2° 2θ, and 24.80±0.2° 2θ.

In some embodiments, the X-ray powder diffraction pattern further comprises peaks at 7.46±0.2° 2θ, 18.14±0.2° 2θ, 20.95±0.2° 2θ, 22.53±0.2° 2θ, and 24.80±0.2° 2θ.

In some embodiments, crystalline compound 1, Form X has an X-ray powder diffraction (XRPD) pattern with characteristic peaks at 7.46±0.2° 2θ, 9.17±0.2° 2θ, 13.44±0.2° 2θ, 14.95±0.2° 2θ, 16.29±0.2° 2θ, 18.14±0.2° 2θ, 20.95±0.2° 2θ, 21.67±0.2° 2θ, 22.53±0.2° 2θ, 23.72±0.2° 2θ, 24.80±0.2° 2, and 25.72±0.2° 2θ.

In some embodiments, crystalline compound 1, Form X has an X-ray powder diffraction (XRPD) pattern with at least five characteristic peaks selected from 7.46±0.2° 2θ, 9.17±0.2° 2θ, 13.44±0.2° 2θ, 14.95±0.2° 2θ, 16.29±0.2° 2θ, 18.14±0.2° 2θ, 20.95±0.2° 2θ, 21.67±0.2° 2θ, 22.53±0.2° 2θ, 23.72±0.2° 2θ, 24.80±0.2° 2, and 25.72±0.2° 2θ.

In some embodiments, crystalline compound 1, Form X has an X-ray powder diffraction (XRPD) pattern with at least six characteristic peaks selected from 7.46±0.2° 2θ, 9.17±0.2° 2θ, 13.44±0.2° 2θ, 14.95±0.2° 2θ, 16.29±0.2° 2θ, 18.14±0.2° 2θ, 20.95±0.2° 2θ, 21.67±0.2° 2θ, 22.53±0.2° 2θ, 23.72±0.2° 2θ, 24.80±0.2° 2, and 25.72±0.2° 2θ.

In some embodiments, crystalline compound 1, Form X has an X-ray powder diffraction (XRPD) pattern with at least seven characteristic peaks selected from 7.46±0.2° 2θ, 9.17±0.2° 2θ, 13.44±0.2° 2θ, 14.95±0.2° 2θ, 16.29±0.2° 2θ, 18.14±0.2° 2θ, 20.95±0.2° 2θ, 21.67±0.2° 2θ, 22.53±0.2° 2θ, 23.72±0.2° 2θ, 24.80±0.2° 2, and 25.72±0.2° 2θ.

In some embodiments, crystalline compound 1, Form X has an X-ray powder diffraction (XRPD) pattern with at least eight characteristic peaks selected from 7.46±0.2° 2θ, 9.17±0.2° 2θ, 13.44±0.2° 2θ, 14.95±0.2° 2θ, 16.29±0.2° 2θ, 18.14±0.2° 2θ, 20.95±0.2° 2θ, 21.67±0.2° 2θ, 22.53±0.2° 2θ, 23.72±0.2° 2θ, 24.80±0.2° 2, and 25.72±0.2° 2θ.

In some embodiments, the X-ray powder diffraction pattern further comprises at least one peak selected from 14.44±0.2° 2θ, 17.15±0.2° 2θ, and 18.74±0.2° 2θ.

In some embodiments, crystalline compound 1, Form X has a DSC thermogram with an endotherm having an onset temperature at about 157° C. and a peak temperature at about 158° C.

In some embodiments, crystalline compound 1, Form X is anhydrous.

In some embodiments, crystalline compound 1, Form X is thermodynamically stable.

In some embodiments, crystalline compound 1, Form X is non-hygroscopic. In some embodiments, crystalline compound 1, Form X is non-hygroscopic with water uptake of 0.11/0.16% at 80/90% RH.

In some embodiments, crystalline compound 1, Form X is physically and chemically stable.

In some embodiments, crystalline compound 1, Form X is physically stable at 40° C./75% RH and 60° C. for at least one week. In some embodiments, crystalline compound 1, Form X is physically stable at 40° C./75% RH and 60° C. for at least two weeks. In some embodiments, crystalline compound 1, Form X is physically stable at 40° C./75% RH and 60° C. for at least three weeks. In some embodiments, crystalline compound 1, Form X is physically stable at 40° C./75% RH and 60° C. for at least four weeks.

TABLE 9
Form X

| 2-Theta | d(Å) | BG | Height | I % | Area | I % | FWHM |
|---|---|---|---|---|---|---|---|
| 3.495 | 25.2596 | 173 | 428 | 10.9 | 5537 | 9.3 | 0.151 |
| 7.102 | 12.4366 | 65 | 127 | 3.2 | 3653 | 6.1 | 0.335 |
| 7.458 | 11.8442 | 65 | 716 | 18.2 | 10497 | 17.6 | 0.171 |
| 9.175 | 9.6307 | 54 | 1830 | 46.5 | 22124 | 37.2 | 0.141 |
| 11.715 | 7.5475 | 48 | 165 | 4.2 | 1779 | 3 | 0.126 |
| 13.238 | 6.6824 | 61 | 317 | 8 | 4391 | 7.4 | 0.162 |
| 13.444 | 6.5809 | 62 | 1190 | 30.2 | 15290 | 25.7 | 0.15 |
| 13.826 | 6.3998 | 68 | 72 | 1.8 | 898 | 1.5 | 0.145 |
| 14.443 | 6.1276 | 81 | 686 | 17.4 | 8892 | 14.9 | 0.151 |
| 14.952 | 5.9203 | 77 | 930 | 23.6 | 13034 | 21.9 | 0.163 |
| 15.459 | 5.7273 | 96 | 405 | 10.3 | 4191 | 7 | 0.121 |
| 15.653 | 5.6567 | 91 | 238 | 6 | 2851 | 4.8 | 0.14 |
| 16.296 | 5.435 | 83 | 1730 | 43.9 | 26150 | 43.9 | 0.176 |
| 17.148 | 5.1667 | 76 | 435 | 11 | 4511 | 7.6 | 0.121 |
| 18.136 | 4.8875 | 82 | 732 | 18.6 | 13177 | 22.1 | 0.21 |
| 18.533 | 4.7836 | 72 | 371 | 9.4 | 18307 | 30.8 | 0.576 |
| 18.738 | 4.7317 | 84 | 483 | 12.3 | 13288 | 22.3 | 0.321 |
| 19.122 | 4.6375 | 89 | 163 | 4.1 | 1238 | 2.1 | 0.089 |
| 19.945 | 4.4481 | 93 | 442 | 11.2 | 4509 | 7.6 | 0.119 |
| 20.386 | 4.3527 | 102 | 326 | 8.3 | 4064 | 6.8 | 0.145 |
| 20.948 | 4.2372 | 121 | 821 | 20.8 | 9836 | 16.5 | 0.14 |
| 21.36 | 4.1565 | 115 | 1131 | 28.7 | 17971 | 30.2 | 0.185 |
| 21.675 | 4.0968 | 111 | 1679 | 42.6 | 22149 | 37.2 | 0.154 |
| 22.527 | 3.9437 | 117 | 1000 | 25.4 | 12627 | 21.2 | 0.147 |
| 23.72 | 3.7479 | 138 | 3938 | 100 | 59527 | 100 | 0.176 |
| 24.145 | 3.6829 | 151 | 457 | 11.6 | 15314 | 25.7 | 0.391 |
| 24.804 | 3.5865 | 179 | 774 | 19.7 | 8250 | 13.9 | 0.124 |
| 25.242 | 3.5253 | 141 | 368 | 9.3 | 5517 | 9.3 | 0.175 |
| 25.722 | 3.4605 | 153 | 1040 | 26.4 | 14285 | 24 | 0.16 |
| 26.314 | 3.3841 | 128 | 259 | 6.6 | 5838 | 9.8 | 0.263 |
| 26.737 | 3.3315 | 119 | 182 | 4.6 | 3540 | 5.9 | 0.227 |
| 27.15 | 3.2817 | 114 | 189 | 4.8 | 4286 | 7.2 | 0.264 |
| 27.489 | 3.242 | 113 | 80 | 2 | 1396 | 2.3 | 0.204 |
| 27.904 | 3.1947 | 110 | 246 | 6.2 | 7723 | 13 | 0.366 |
| 28.343 | 3.1463 | 115 | 175 | 4.4 | 7757 | 13 | 0.517 |
| 29.07 | 3.0692 | 111 | 348 | 8.8 | 6189 | 10.4 | 0.207 |
| 29.548 | 3.0206 | 104 | 56 | 1.4 | 664 | 1.1 | 0.138 |
| 29.88 | 2.9878 | 107 | 55 | 1.4 | 667 | 1.1 | 0.141 |
| 30.168 | 2.9599 | 113 | 112 | 2.8 | 2449 | 4.1 | 0.255 |
| 30.564 | 2.9224 | 122 | 82 | 2.1 | 1312 | 2.2 | 0.187 |
| 30.965 | 2.8856 | 124 | 175 | 4.4 | 2660 | 4.5 | 0.177 |
| 31.255 | 2.8594 | 122 | 176 | 4.5 | 5408 | 9.1 | 0.358 |
| 31.553 | 2.8331 | 121 | 108 | 2.7 | 2554 | 4.3 | 0.276 |
| 32.02 | 2.7929 | 116 | 401 | 10.2 | 6277 | 10.5 | 0.183 |
| 32.434 | 2.7581 | 105 | 356 | 9 | 8251 | 13.9 | 0.27 |
| 33.624 | 2.6632 | 83 | 45 | 1.1 | 753 | 1.3 | 0.195 |
| 33.862 | 2.645 | 81 | 43 | 1.1 | 758 | 1.3 | 0.206 |
| 34.422 | 2.6033 | 94 | 94 | 2.4 | 939 | 1.6 | 0.117 |
| 35.204 | 2.5472 | 94 | 180 | 4.6 | 2103 | 3.5 | 0.136 |
| 36.193 | 2.4798 | 82 | 145 | 3.7 | 3581 | 6 | 0.288 |
| 37.359 | 2.4051 | 95 | 89 | 2.3 | 2364 | 4 | 0.31 |
| 37.757 | 2.3806 | 96 | 140 | 3.6 | 3378 | 5.7 | 0.281 |
| 39.004 | 2.3073 | 85 | 95 | 2.4 | 3079 | 5.2 | 0.378 |

### Preparation of Crystalline Compound 1

In some embodiments, crystalline forms of compound 1 are prepared as outlined in the Examples. It is noted that solvents, temperatures, and other reaction conditions presented herein may vary.

In some embodiments, provided herein are methods for making a solid form of compound 1, comprising 1) suspending compound 1 in a solvent at a first temperature (e.g., ambient temperature); 2) cycling the compound 1 mixture between ambient and a second temperature (e.g., about 40° C.); 3) collecting a solid if there is precipitation, or evaporating the solvent to collect a solid if there is no precipitation; and 4) optionally drying. In some embodiments, provided herein are methods for making a solid form of compound 1, comprising 1) obtaining a saturated solution of compound 1 in a solvent; 2) adding an anti-solvent into the saturated solution; 3) cooling down to about 2-8° C. and about −20° C.; 4) collecting a solid if there is precipitation, or evaporating the solvent to collect a solid if there is no precipitation; and 5) optionally drying. In some embodiments, the ratio by volume of solvent and anti-solvent is about 1:9. In some embodiments, the ratio by volume of solvent and anti-solvent is about 1:4. In some embodiments, the ratio by volume of solvent and anti-solvent is about 1:2. In some embodiments, the ratio by volume of solvent and anti-solvent is about 1:1. In some embodiments, the methods for making a solid form of compound 1 are anti-solvent recrystallization experiments.

In another embodiment, crystalline compound 1 is substantially pure. In some embodiments, the substantially pure crystalline compound 1. In some embodiments, the pure crystalline compound 1 is substantially free of other solid forms, e.g., amorphous solid. In some embodiments, the purity of the substantially pure crystalline compound 1 is no less than about 95%, no less than about 96%, no less than about 97%, no less than about 98%, no less than about 98.5%, no less than about 99%, no less than about 99.5%, or no less than about 99.8%. In some embodiments, the purity of the substantially pure crystalline compound 1 is about 95%, about 96%, about 97%, about 98%, about 98.5%, about 99%, about 99.5%, or about 99.8%.

### Method of Treatment

Described herein is compound 1, or a pharmaceutically acceptable salt or solvate thereof, generally useful for the inhibition of kinase activity of one or more enzymes. Examples of kinases that are inhibited by compound 1, or a pharmaceutically acceptable salt or solvate thereof, and compositions described herein and against which the methods described herein are useful include p38 MAP kinase, MK2, or a mutant thereof.

MAP kinase-activated protein kinase 2 (“MK2”) is an enzyme that in humans is encoded by the MAPKAPK2 gene. This gene encodes a member of the Ser/Thr protein kinase family. This kinase is regulated through direct phosphorylation by p38 MAP kinase. In conjunction with p38 MAP kinase, this kinase is known to be involved in many cellular processes including stress and inflammatory responses, nuclear export, gene expression regulation and cell proliferation. Heat shock protein HSP27 was shown to be one of the substrates of this kinase in vivo. Two transcript variants encoding two different isoforms have been found for this gene.

MK2 is a multi-domain protein consisting of an N-terminal proline-rich domain, a catalytic domain, an autoinhibitory domain and at the C-terminus a nuclear export signal (NES) and nuclear localization signal (NLS). Two isoforms of human MK2 have been characterized. One isoform consists of 400 amino acids and the other isoform 370 residues which is thought to be a splice variant missing the C-terminal NLS. MK2 is located in the nucleus of the cell and upon binding and phosphorylation by p38, the MK2 NES becomes functional and both kinases are co-transported out of the nucleus to the cytoplasm. Interestingly, transport of the MK2/p38 complex does not require catalytically active MK2, as the active site mutant, Asp207Ala, is still transported to the cytoplasm. Phosphorylation of human MK2 by p38 on residues T222, S272 and T334 is thought to activate the enzyme by inducing a conformational change of the autoinhibitory domain thus exposing the active site for substrate binding. Mutations of two autoinhibitory domain residues W332A and K326E in murine MK2 demonstrate an increase in basal activity and a C-terminal deletion of the autoinhibitory domain renders the enzyme constitutively active, providing additional evidence to the role of this domain in inhibition of MK2 activity.

Diseases or disorders associated with MK2 that are treated by compound 1, or a pharmaceutically acceptable salt or solvate thereof, include autoimmune disorders, chronic inflammatory disorders, acute inflammatory disorders, auto-inflammatory disorders, fibrotic disorders, metabolic disorders, neoplastic disorders, and cardiovascular or cerebrovascular disorders.

In some embodiments, the MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic purpura, metal-induced autoimmunity, myasthenia gravis, pemphigus, autoimmune deafness (for example, Meniere's disease), Goodpasture's syndrome, Graves' disease, HW-related autoimmune syndromes, Guillain-Barre disease, Addison's disease, anti-phospholipid syndrome, asthma, atopic dermatitis, Celiac disease, Cushing's syndrome, dermatomyositis, idiopathic adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post-surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, the MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, the MK2-mediated disease or disorder is a metabolic disorder. Exemplary metabolic disorders include obesity, steroid-resistance, glucose intolerance, and metabolic syndrome.

In some embodiments, the MK2-mediated disease or disorder is a neoplastic disease or disorder. Exemplary neoplastic diseases or disorders include cancers. In some embodiments, exemplary neoplastic diseases or disorders include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, the MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia, and peripheral neuropathy.

### Dosing

In certain embodiments, the compositions containing compound 1, or a pharmaceutically acceptable salt or solvate thereof, are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of compound 1, or a pharmaceutically acceptable salt or solvate thereof, including further embodiments in which (i) the compound is administered once a day; or (ii) the compound is administered to the mammal multiple times over the span of one day.

In any of the aforementioned aspects are further embodiments comprising multiple administrations of the effective amount of compound 1, or a pharmaceutically acceptable salt or solvate thereof, including further embodiments in which (i) the compound is administered continuously or intermittently: as in a single dose; (ii) the time between multiple administrations is every 6 hours; (iii) the compound is administered to the mammal every 8 hours; (iv) the compound is administered to the subject every 12 hours; (v) the compound is administered to the subject every 24 hours.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, compound 1, or a pharmaceutically acceptable salt or solvate thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

In some embodiments, compound 1, or a pharmaceutically acceptable salt or solvate thereof, is administered to a subject in need thereof, either alone or in combination with pharmaceutically acceptable carriers, excipients, or diluents, in a pharmaceutical composition, according to standard pharmaceutical practice. In one embodiment, compound 1, or a pharmaceutically acceptable salt or solvate thereof, may be administered to animals. Compound 1, or a pharmaceutically acceptable salt or solvate thereof, can be administered orally or parenterally, including the intravenous, intramuscular, intraperitoneal, subcutaneous, rectal, and topical routes of administration.

In another aspect, provided herein are pharmaceutical compositions comprising compound 1, or a pharmaceutically acceptable salt or solvate thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compound 1, or a pharmaceutically acceptable salt or solvate thereof, are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, N-oxide, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

In some embodiments, the pharmaceutical composition comprises compound 1, wherein in at least 95%, or at least 97%, or at least 99% of compound 1 is Form I of compound 1. In some embodiments, the pharmaceutical composition comprises compound 1, wherein in at least 95%, or at least 97%, or at least 99% of compound 1 is Form II of compound 1. In some embodiments, the pharmaceutical composition comprises compound 1, wherein in at least 95%, or at least 97%, or at least 99% of compound 1 is Form III of compound 1. In some embodiments, the pharmaceutical composition comprises compound 1, wherein in at least 95%, or at least 97%, or at least 99% of compound 1 is Form IV of compound 1. In some embodiments, the pharmaceutical composition comprises compound 1, wherein in at least 95%, or at least 97%, or at least 99% of compound 1 is Form VI of compound 1. In some embodiments, the pharmaceutical composition comprises compound 1, wherein in at least 95%, or at least 97%, or at least 99% of compound 1 is Form VII of compound 1. In some embodiments, the pharmaceutical composition comprises compound 1, wherein in at least 95%, or at least 97%, or at least 99% of compound 1 is Form VIII of compound I. In some embodiments, the pharmaceutical composition comprises compound 1, wherein in at least 95%, or at least 97%, or at least 99% of compound 1 is Form IX of compound 1. In some embodiments, the pharmaceutical composition comprises compound 1, wherein in at least 95%, or at least 97%, or at least 99% of compound 1 is and Form X of compound 1.

In some embodiments, the pharmaceutical compositioncomprises compound 1, wherein in at least 95%, or at least 97%, or at least 99% of compound 1 is freebase Form I of compound 1. In some embodiments, the pharmaceutical composition comprises compound 1, wherein in at least 95%, or at least 97%, or at least 99% of compound 1 is freebase Form II of compound 1. In some embodiments, the pharmaceutical composition comprises compound 1, wherein in at least 95%, or at least 97%, or at least 99% of compound 1 is freebase Form III of compound 1. In some embodiments, the pharmaceutical composition comprises compound 1, wherein in at least 95%, or at least 97%, or at least 99% of compound 1 is freebase Form IV of compound 1. In some embodiments, the pharmaceutical composition comprises compound 1, wherein in at least 95%, or at least 97%, or at least 99% of compound 1 is freebase Form VI of compound 1. In some embodiments, the pharmaceutical composition comprises compound 1, wherein in at least 95%, or at least 97%, or at least 99% of compound 1 is freebase Form VII of compound 1. In some embodiments, the pharmaceutical composition comprises compound 1, wherein in at least 95%, or at least 97%, or at least 99% of compound 1 is freebase Form VIII of compound 1. In some embodiments, the pharmaceutical composition comprises compound 1, wherein in at least 95%, or at least 97%, or at least 99% of compound 1 is freebase Form IX of compound 1. In some embodiments, the pharmaceutical composition comprises compound 1, wherein in at least 95%, or at least 97%, or at least 99% of compound 1 is and freebase Form X of compound 1

### Combination

Disclosed herein are methods of treating an autoimmune disorder, a chronic inflammatory disorder, an acute inflammatory disorder, an auto-inflammatory disorder, a fibrotic disorder, a metabolic disorder, a neoplastic disorder, or a cardiovascular or a cerebrovascular disorder using compound 1, or a pharmaceutically acceptable salt or solvate thereof, in combination with an additional therapeutic agent.

In some embodiments, the additional therapeutic agent is selected from the group consisting of anti-inflammatory drugs, anti-atherosclerotic drugs, immunosuppressive drugs, immunomodulatory drugs, cytostatic drugs, anti-proliferative agents, angiogenesis inhibitors, kinase inhibitors, cytokine blockers, and inhibitors of cell adhesion molecules.

In some embodiments, the additional therapeutic agent is selected from the group consisting of NSAIDs, immunosuppressive drugs, immunomodulatory drugs, cytostatic drugs, antiproliferative agents, angiogenesis inhibitors, biological agents, steroids, vitamin D3 analogs, retinoids, other kinase inhibitors, cytokine blockers, corticosteroids, and inhibitors of cell adhesion molecules. In some embodiments, the additional therapeutic agent is selected from the group consisting of torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol, and clofibrate.

In some embodiments, the additional therapeutic agent is selected from the group consisting of corticosteroids, nonsteroidal anti-inflammatory drugs (NSAID) (e.g. ibuprofen, naproxen, acetaminophen, aspirin, Fenoprofen (Nalfon), Flurbiprofen (Ansaid), Ketoprofen, Oxaprozin (Daypro), Diclofenac sodium (Voltaren), Diclofenac potassium (Cataflam), Etodolac (Lodine), Indomethacin (Indocin), Ketorolac (Toradol), Sulindac (Clinoril), Tolmetin (Tolectin), Meclofenamate (Meclomen), Mefenamic acid (Ponstel), Nabumetone (Relafen), Piroxicam (Feldene), cox-2 inhibitors (e.g., celecoxib (Celebrex))), immunosuppressants (e.g., methotrexate (Rheumatrex), leflunomide (Arava), azathioprine (Imuran), cyclosporine (Neoral, Sandimmune), tacrolimus and cyclophosphamide (Cytoxan), CD20 blockers (Rituximab), Tumor Necrosis Factor (TNF) blockers (e.g., etanercept (Enbrel), infliximab (Remicade) and adalimumab (Humira)), Abatacept (CTLA4-Ig) and interleukin-1 receptor antagonists (e.g. Anakinra (Kineret), interleukin 6 inhibitors (e.g., Actemra), interleukin 17 inhibitors (e.g., AIN457), Janus kinase inhibitors (e.g., Tasocitinib), syk inhibitors (e.g. R788), and chloroquine and its derivatives.

In some embodiments, the additional therapeutic agent is selected from the group consisting of an EGFR kinase inhibitor, MEK inhibitor, VEGFR inhibitor, anti-VEGFR2 antibody, KDR antibody, AKT inhibitor, PDK-1 inhibitor, PI3K inhibitor, c-kit/Kdr tyrosine kinase inhibitor, Bcr-Abl tyrosine kinase inhibitor, VEGFR2 inhibitor, PDGFR-beta inhibitor, KIT inhibitor, Flt3 tyrosine kinase inhibitor, PDGF receptor family inhibitor, Flt3 tyrosine kinase inhibitor, RET tyrosine kinase receptor family inhibitor, VEGF-3 receptor antagonist, Raf protein kinase family inhibitor, angiogenesis inhibitor, Erb2 inhibitor, mTOR inhibitor, IGF-1R antibody, NFkB inhibitor, proteosome inhibitor, chemotherapy agent, and glucose reduction agent.

In some embodiments, the additional therapeutic agent is administered at the same time as compound 1, or a pharmaceutically acceptable salt or solvate thereof. In some embodiments, the additional therapeutic agent and compound 1, or a pharmaceutically acceptable salt or solvate thereof, are administered sequentially. In some embodiments, the additional therapeutic agent is administered less frequently than compound 1, or a pharmaceutically acceptable salt or solvate thereof. In some embodiments, the additional therapeutic agent is administered more frequently than compound 1, or a pharmaceutically acceptable salt or solvate thereof. In some embodiments, the additional therapeutic agent is administered prior to the administration of compound 1, or a pharmaceutically acceptable salt or solvate thereof. In some embodiments, the additional therapeutic agent is administered after the administration of compound 1, or a pharmaceutically acceptable salt or solvate thereof.

### EXAMPLES

### Example 1: Single Crystal Structure Determination

The objective of this study was to grow single crystal of compoundl and determine its structure by single crystal X-ray diffraction.

### Single Crystal Growth

About 30.4 mg of compound 1 was dissolved in 1.0 mL of toluene by stirring at 50° C. After removal of the stir bar, the solution was kept at room temperature for cooling crystallization. After 2 days, block shaped single crystals were obtained and used for single crystal X-ray diffraction.

### Instruments and Parameters

Single crystal X-ray diffraction data of compound 1 was collected at 180 K on a Rigaku XtaLAB PRO 007HF(Mo) diffractometer, with Mo Ka radiation (λ=0.71073 Å). Data reduction and empirical absorption correction were performed using the CrysAlisPro program. The structure was solved by a dual-space algorithm using SHELXT program. All non-hydrogen atoms could be located directly from the difference Fourier maps. Framework hydrogen atoms were placed geometrically and constrained using the riding model to the parent atoms. Final structure refinement was done using the SHELXL program by minimizing the sum of squared deviations of F² using a full-matrix technique.

### Single Crystal X-ray Diffraction Analysis

Compound 1 crystallized as orthorhombic in P2₁2₁2₁ space group with formula of C₂₄H_{22.52}N₅O_{3.76}F₃Cl (FIG. 1). There is one compound 1 molecule and 0.76 water molecule in each asymmetric unit, and the unit cell contains four asymmetric units. FIG. 1 illustrates that the axial chirality is determined unambiguously.

The crystal structural data are summarized in Table 10, and the details on atomic coordinates, anisotropic displacement parameters, bond lengths and angles, hydrogen bonds, torsion angles and atomic occupancy are presented in Table 11a to Table 11i.

TABLE 11a
Crystal Data and Structure Refinement for Compound 1

| Empirical formula | C₂₄H_{22.52}N₅O_{3.76}F₃Cl |
|---|---|
| Formula weight | 533.60 |
| Temperature/K | 180.00(10) |
| Crystal system | orthorhombic |
| Space group | P2₁2₁2₁ |
| a/Å | 12.55590(10) |
| b/Å | 13.67040(10) |
| c/Å | 14.3365(2) |
| α/° | 90 |
| β/° | 90 |
| γ/° | 90 |
| Volume/Å³ | 2460.78(4) |
| Z | 4 |
| ρ_{calc}g/cm³ | 1.440 |
| μ/mm⁻¹ | 0.218 |
| F(000) | 1102.0 |
| Crystal size/mm³ | 0.18 × 0.07 × 0.06 |
| Radiation | Mo Kα (λ = 0.71073 Å) |
| 2θ range for data collection/° | 4.116 to 59.046 |
| Index ranges | −17 ≤ h ≤ 16, −18 ≤ k ≤ 18, −19 ≤ |
| | l ≤ 19 |
| Reflections collected | 59756 |
| Independent reflections | 6525 [Rᵢₙₜ = 0.0273, R_{sigma} = 0.0162] |
| Data/restraints/parameters | 6525/25/362 |
| Goodness-of-fit on F² | 1.044 |
| Final R indexes [I >= 2σ (I)] | R₁ = 0.0485, wR₂ = 0.1393 |
| Final R indexes [all data] | R₁ = 0.0521, wR₂ = 0.1426 |
| Largest diff. peak/hole/e Å⁻³ | 1.14/−0.59 |
| Flack parameter | 0.013(9) |

TABLE 11b
Fractional Atomic Coordinates (×10⁴) and
Equivalent Isotropic Displacement
Parameters (Å² × 10³) for Compound 1

| Atom | x | y | z | U(eq) |
|---|---|---|---|---|
| C11 | 4044.6(5) | 2890.8(5) | 1681.1(5) | 30.86(17) |
| F1A | −237(3) | 5386(4) | 4750(4) | 80.3(15) |
| F1B | −509(6) | 5678(8) | 4065(8) | 80.3(15) |
| F2A | 3524(3) | 5288(5) | 4938(3) | 64.9(12) |
| F2B | 3146(8) | 5472(10) | 4800(8) | 64.9(12) |
| F3 | 8213.4(15) | 3786.1(16) | 3111.3(11) | 39.4(4) |
| O1 | 3882.2(15) | 4800.7(15) | 2514.0(16) | 32.5(5) |
| O2 | 6323.5(17) | 2623.0(14) | 1157.6(15) | 30.5(4) |
| O3A | 11068(4) | 3196(4) | 5948(3) | 67.3(12) |
| O3B | 10359(11) | 1132(11) | 4977(9) | 67.3(12) |
| N1A | 1755(3) | 5800(4) | 2994(2) | 39.0(11) |
| C1A | 2679(2) | 5653(4) | 3509(3) | 32.2(9) |
| C2A | 2614(2) | 5423(4) | 4452(3) | 32.4(11) |
| C3A | 1624(3) | 5340(3) | 4879(2) | 50.8(16) |
| C4A | 700(2) | 5488(3) | 4364(3) | 52.8(16) |
| C5A | 766(2) | 5717(3) | 3421(3) | 51.3(16) |
| N1B | 1833(6) | 5825(9) | 2647(5) | 39.0(11) |
| C1B | 2603(4) | 5650(10) | 3324(6) | 32.2(9) |
| C2B | 2302(6) | 5498(8) | 4245(5) | 32.4(11) |
| C3B | 1231(7) | 5521(7) | 4491(5) | 50.8(16) |
| C4B | 461(4) | 5696(7) | 3814(7) | 52.8(16) |
| C5B | 762(5) | 5848(7) | 2893(6) | 51.3(16) |
| N2 | 6912.7(17) | 4124.0(15) | 1647.7(16) | 23.2(4) |
| N3 | 10099.7(19) | 3213(2) | 1297.3(18) | 32.7(5) |
| N4 | 10347.7(19) | 3141.5(19) | 2907.5(18) | 31.3(5) |
| N5 | 9955(2) | 2793(2) | 3730.2(19) | 39.4(6) |
| C6 | 3714(2) | 5730(2) | 3000(2) | 36.9(7) |
| C7 | 4875(2) | 4617.3(18) | 2210.1(18) | 24.7(5) |
| C8 | 5722(2) | 5303.8(19) | 2271(2) | 27.6(5) |
| C9 | 6723(2) | 5047.3(19) | 2007.5(18) | 25.5(5) |
| C10 | 6106(2) | 3418.6(17) | 1504.4(17) | 22.7(5) |
| C11 | 5066(2) | 3718.8(18) | 1813.8(17) | 23.5(5) |
| C12 | 7636(2) | 5755(2) | 2068(2) | 34.6(6) |
| C13 | 7989(2) | 3799.1(19) | 1486.5(17) | 24.3(5) |
| C14 | 8418(2) | 3691(2) | 595.6(19) | 27.9(5) |
| C15 | 9484(2) | 3396(2) | 557(2) | 33.1(6) |
| C16 | 9671(2) | 3316(2) | 2141(2) | 28.2(5) |
| C17 | 8622(2) | 3617(2) | 2260.7(19) | 27.3(5) |
| C18 | 7780(3) | 3884(3) | −267(2) | 36.2(6) |
| C19 | 11417(2) | 3316(2) | 2962(2) | 33.0(6) |
| C20 | 11717(2) | 3066(2) | 3839(3) | 38.8(7) |
| C21 | 10786(3) | 2749(3) | 4298(2) | 40.4(7) |
| C22A | 10650(4) | 2439(5) | 5340(3) | 46.2(12) |
| C22B | 10682(14) | 2115(13) | 5106(11) | 46.2(12) |
| C23A | 9488(4) | 2341(6) | 5606(4) | 61.7(18) |
| C23B | 9880(14) | 2677(16) | 5650(14) | 61.7(18) |
| C24A | 11255(5) | 1489(5) | 5475(4) | 60.4(15) |
| C24B | 11704(13) | 2121(15) | 5657(12) | 60.4(15) |
| O4A | 13142(4) | 3453(4) | 6156(4) | 74.1(14) |

U(eq) is defined as one third of the trace of the orthogonalized Uᵢⱼ tensor.

TABLE 11c
Anisotropic Displacement Parameters (Å² × 10³) for Compound 1

| Atom | U₁₁ | U₂₂ | U₃₃ | U₂₃ | U₁₃ | U₁₂ |
|---|---|---|---|---|---|---|
| C11 | 26.9(3) | 25.6(3) | 40.1(3) | −5.8(2) | 1.2(3) | −5.9(2) |
| F1A | 39.1(19) | 103(4) | 99(3) | −27(3) | 35(2) | −4(2) |
| F1B | 39.1(19) | 103(4) | 99(3) | −27(3) | 35(2) | −4(2) |
| F2A | 36(3) | 104(3) | 55(2) | 11(2) | −17(2) | −3(3) |
| F2B | 36(3) | 104(3) | 55(2) | 11(2) | −17(2) | −3(3) |
| F3 | 34.9(9) | 61.6(12) | 21.8(8) | 6.3(8) | 5.8(7) | 10.7(8) |
| O1 | 23.3(9) | 29.1(9) | 45.0(11) | −12.7(8) | 6.0(8) | −0.6(7) |
| O2 | 31.5(9) | 22.3(9) | 37.8(10) | −2.8(7) | 5.8(8) | 2.1(7) |
| O3A | 58(2) | 100(3) | 43.9(18) | −25(2) | −12.6(17) | 10(2) |
| O3B | 58(2) | 100(3) | 43.9(18) | −25(2) | −12.6(17) | 10(2) |
| N1A | 28.7(15) | 41.3(16) | 47(3) | −2(3) | 3(2) | 8.6(13) |
| C1A | 26.3(14) | 26.1(13) | 44(3) | −14.0(18) | 5.5(15) | −0.6(11) |
| C2A | 15(3) | 45(2) | 38(3) | −13(2) | −8(2) | 4(2) |
| C3A | 53(4) | 48(3) | 52(4) | −15(3) | 23(3) | −2(3) |
| C4A | 36(3) | 55(3) | 68(4) | −22(3) | 20(3) | −4(2) |
| C5A | 27.6(18) | 49(3) | 77(5) | −14(3) | 0(3) | 4.9(17) |
| N1B | 28.7(15) | 41.3(16) | 47(3) | −2(3) | 3(2) | 8.6(13) |
| C1B | 26.3(14) | 26.1(13) | 44(3) | −14.0(18) | 5.5(15) | −0.6(11) |
| C2B | 15(3) | 45(2) | 38(3) | −13(2) | −8(2) | 4(2) |
| C3B | 53(4) | 48(3) | 52(4) | −15(3) | 23(3) | −2(3) |
| C4B | 36(3) | 55(3) | 68(4) | −22(3) | 20(3) | −4(2) |
| C5B | 27.6(18) | 49(3) | 77(5) | −14(3) | 0(3) | 4.9(17) |
| N2 | 22.2(9) | 22.9(9) | 24.5(10) | −0.1(8) | 3.1(8) | 0.3(7) |
| N3 | 25.5(11) | 40.1(13) | 32.5(12) | −2.9(10) | 4.1(9) | 1.1(10) |
| N4 | 24.2(11) | 36.5(12) | 33.2(12) | 2.4(9) | −2.1(9) | −1.4(9) |
| N5 | 31.5(13) | 51.7(16) | 35.0(13) | 10.9(12) | −3.3(11) | −3.1(12) |
| C6 | 27.1(13) | 31.0(14) | 52.5(18) | −15.0(13) | 8.1(12) | −1.8(11) |
| C7 | 22.7(11) | 22.5(11) | 29.0(12) | −1.7(9) | 0.8(9) | −0.5(9) |
| C8 | 27.1(12) | 22.8(11) | 32.9(13) | −3.8(10) | 2.6(10) | −3.1(9) |
| C9 | 28.3(12) | 22.2(11) | 26.1(11) | −1.3(9) | 3.6(10) | −4.9(9) |
| C10 | 24.1(12) | 21.2(10) | 22.8(10) | 2.9(8) | 1.3(9) | 0.0(9) |
| C11 | 22.9(11) | 22.4(11) | 25.0(11) | −0.3(9) | −0.6(9) | −2.9(9) |
| C12 | 31.2(13) | 27.8(13) | 44.9(16) | −5.3(11) | 9.8(12) | −9.3(11) |
| C13 | 24.4(11) | 25.0(11) | 23.5(11) | 0.9(9) | 4.4(9) | −0.4(9) |
| C14 | 27.6(12) | 32.0(13) | 24.2(12) | −2.3(10) | 3.4(10) | 0.3(10) |
| C15 | 29.2(13) | 44.3(16) | 25.8(13) | −5.0(11) | 5.7(11) | 1.3(12) |
| C16 | 24.8(12) | 31.0(13) | 28.7(12) | 0.7(10) | 0.5(10) | −1.5(10) |
| C17 | 26.2(12) | 32.2(12) | 23.3(11) | 2.2(10) | 4.8(9) | −0.1(10) |
| C18 | 35.5(15) | 48.2(17) | 25.0(12) | −3.7(12) | 0.6(11) | 5.4(13) |
| C19 | 23.6(12) | 29.2(13) | 46.1(16) | −3.5(12) | 0.6(11) | 3.0(10) |
| C20 | 27.3(13) | 38.3(16) | 50.6(18) | −1.9(13) | −8.7(13) | 4.0(12) |
| C21 | 32.8(15) | 44.2(17) | 44.1(17) | 7.9(14) | −7.5(13) | 4.5(13) |
| C22A | 42.9(19) | 68(4) | 27(2) | 1(2) | −5.6(19) | 8(2) |
| C22B | 42.9(19) | 68(4) | 27(2) | 1(2) | −5.6(19) | 8(2) |
| C23A | 37(3) | 114(6) | 34(2) | 15(3) | 4(2) | 10(3) |
| C23B | 37(3) | 114(6) | 34(2) | 15(3) | 4(2) | 10(3) |
| C24A | 60(3) | 79(4) | 43(2) | 23(3) | −3(2) | 16(3) |
| C24B | 60(3) | 79(4) | 43(2) | 23(3) | −3(2) | 16(3) |
| O4A | 60(3) | 90(4) | 72(3) | −4(3) | −8(2) | 15(2) |

The Anisotropic displacement factor exponent takes the form: −2π²[h²a*²U₁₁ + 2hka*b*U₁₂ + . . . ].

TABLE 11d
Bond Lengths for Compound 1

| Atom | Atom | Length/Å |
|---|---|---|
| C11 | C11 | 1.721(3) |
| F1A | C4A | 1.308(4) |
| F1B | C4B | 1.270(9) |
| F2A | C2A | 1.351(4) |
| F2B | C2B | 1.325(9) |
| F3 | C17 | 1.343(3) |
| O1 | C6 | 1.464(3) |
| O1 | C7 | 1.344(3) |
| O2 | C10 | 1.227(3) |
| O3A | C22A | 1.451(7) |
| O3B | C22B | 1.417(19) |
| N1A | C1A | 1.3900 |
| N1A | C5A | 1.3900 |
| C1A | C2A | 1.3900 |
| C1A | C6 | 1.494(4) |
| C2A | C3A | 1.3900 |
| C3A | C4A | 1.3900 |
| C4A | C5A | 1.3900 |
| N1B | C1B | 1.3900 |
| N1B | C5B | 1.3900 |
| C1B | C2B | 1.3900 |
| C1B | C6 | 1.474(6) |
| C2B | C3B | 1.3900 |
| C3B | C4B | 1.3900 |
| C4B | C5B | 1.3900 |
| N2 | C9 | 1.384(3) |
| N2 | C10 | 1.413(3) |
| N2 | C13 | 1.441(3) |
| N3 | C15 | 1.336(4) |
| N3 | C16 | 1.332(4) |
| N4 | N5 | 1.364(4) |
| N4 | C16 | 1.409(3) |
| N4 | C19 | 1.366(4) |
| N5 | C21 | 1.325(4) |
| C7 | C8 | 1.421(3) |
| C7 | C11 | 1.375(3) |
| C8 | C9 | 1.359(4) |
| C9 | C12 | 1.503(4) |
| C10 | C11 | 1.439(3) |
| C13 | C14 | 1.394(3) |
| C13 | C17 | 1.388(4) |
| C14 | C15 | 1.399(4) |
| C14 | C18 | 1.497(4) |
| C16 | C17 | 1.390(4) |
| C19 | C20 | 1.356(5) |
| C20 | C21 | 1.410(5) |
| C21 | C22A | 1.562(6) |
| C21 | C22B | 1.452(17) |
| C22A | C23A | 1.513(8) |
| C22A | C24A | 1.518(8) |
| C22B | C23B | 1.487(19) |
| C22B | C24B | 1.506(19) |

TABLE 11e
Bond Angles for Compound 1

| Atom | Atom | Atom | Angle/° |
|---|---|---|---|
| C7 | O1 | C6 | 116.7(2) |
| C1A | N1A | C5A | 120.0 |
| N1A | C1A | C2A | 120.0 |
| N1A | C1A | C6 | 117.1(3) |
| C2A | C1A | C6 | 122.8(3) |
| F2A | C2A | C1A | 118.8(3) |
| F2A | C2A | C3A | 121.2(3) |
| C1A | C2A | C3A | 120.0 |
| C4A | C3A | C2A | 120.0 |
| F1A | C4A | C3A | 120.7(4) |
| F1A | C4A | C5A | 119.3(4) |
| C5A | C4A | C3A | 120.0 |
| C4A | C5A | N1A | 120.0 |
| C1B | N1B | C5B | 120.0 |
| N1B | C1B | C6 | 115.2(6) |
| C2B | C1B | N1B | 120.0 |
| C2B | C1B | C6 | 124.6(6) |
| F2B | C2B | C1B | 110.9(7) |
| F2B | C2B | C3B | 128.5(7) |
| C3B | C2B | C1B | 120.0 |
| C2B | C3B | C4B | 120.0 |
| F1B | C4B | C3B | 117.8(8) |
| F1B | C4B | C5B | 122.2(8) |
| C5B | C4B | C3B | 120.0 |
| C4B | C5B | N1B | 120.0 |
| C9 | N2 | C10 | 123.6(2) |
| C9 | N2 | C13 | 120.2(2) |
| C10 | N2 | C13 | 116.0(2) |
| C16 | N3 | C15 | 117.9(2) |
| N5 | N4 | C16 | 121.0(2) |
| N5 | N4 | C19 | 111.5(3) |
| C19 | N4 | C16 | 127.4(3) |
| C21 | N5 | N4 | 105.2(3) |
| O1 | C6 | C1A | 107.3(3) |
| O1 | C6 | C1B | 102.8(5) |
| O1 | C7 | C8 | 123.4(2) |
| O1 | C7 | C11 | 117.6(2) |
| C11 | C7 | C8 | 119.0(2) |
| C9 | C8 | C7 | 120.3(2) |
| N2 | C9 | C12 | 118.5(2) |
| C8 | C9 | N2 | 119.9(2) |
| C8 | C9 | C12 | 121.6(2) |
| O2 | C10 | N2 | 120.3(2) |
| O2 | C10 | C11 | 125.4(2) |
| N2 | C10 | C11 | 114.2(2) |
| C7 | C11 | C11 | 120.18(19) |
| C7 | C11 | C10 | 122.8(2) |
| C10 | C11 | C11 | 117.05(18) |
| C14 | C13 | N2 | 122.8(2) |
| C17 | C13 | N2 | 117.7(2) |
| C17 | C13 | C14 | 119.5(2) |
| C13 | C14 | C15 | 115.9(3) |
| C13 | C14 | C18 | 122.1(2) |
| C15 | C14 | C18 | 122.0(3) |
| N3 | C15 | C14 | 125.1(3) |
| N3 | C16 | N4 | 116.5(2) |
| N3 | C16 | C17 | 121.7(3) |
| C17 | C16 | N4 | 121.7(2) |
| F3 | C17 | C13 | 118.4(2) |
| F3 | C17 | C16 | 121.7(2) |
| C13 | C17 | C16 | 119.8(2) |
| C20 | C19 | N4 | 106.4(3) |
| C19 | C20 | C21 | 106.2(3) |
| N5 | C21 | C20 | 110.7(3) |
| N5 | C21 | C22A | 120.9(3) |
| N5 | C21 | C22B | 116.5(8) |
| C20 | C21 | C22A | 128.3(3) |
| C20 | C21 | C22B | 129.0(8) |
| O3A | C22A | C21 | 110.0(5) |
| O3A | C22A | C23A | 105.1(5) |
| O3A | C22A | C24A | 110.6(5) |
| C23A | C22A | C21 | 111.8(4) |
| C23A | C22A | C24A | 112.0(6) |
| C24A | C22A | C21 | 107.4(4) |
| O3B | C22B | C21 | 119.2(13) |
| O3B | C22B | C23B | 111.3(15) |
| O3B | C22B | C24B | 108.5(13) |
| C21 | C22B | C23B | 99.9(13) |
| C21 | C22B | C24B | 109.8(13) |
| C23B | C22B | C24B | 107.4(14) |

TABLE 11f
Hydrogen Bonds for Compound 1

| D | H | A | d(D-H)/Å | d(H-A)/Å | d(D-A)/Å | D-H-A/º |
|---|---|---|---|---|---|---|
| O3A | H3C | O4A | 0.82 | 1.85 | 2.645(7) | 163.0 |
| O4A | H4C | F2A¹ | 1.00(3) | 2.10(3) | 3.094(8) | 174(7) |
| O4A | H4D | N5² | 0.95(3) | 1.94(4) | 2.847(6) | 159(7) |

¹1 + x, +y, +z;
²1/2 + x, 1/2 − y, 1 − z

TABLE 11g
Torsion Angles for Compound 1

| A | B | C | D | Angle/° |
|---|---|---|---|---|
| F1A | C4A | C5A | N1A | −178.1(5) |
| F1B | C4B | C5B | N1B | −177.6(11) |
| F2A | C2A | C3A | C4A | 179.7(5) |
| F2B | C2B | C3B | C4B | 170.3(12) |
| O1 | C7 | C8 | C9 | 175.6(3) |
| O1 | C7 | C11 | C11 | 2.4(3) |
| O1 | C7 | C11 | C10 | −176.9(2) |
| O2 | C10 | C11 | C11 | −0.2(4) |
| O2 | C10 | C11 | C7 | 179.0(3) |
| N1A | C1A | C2A | F2A | −179.7(5) |
| N1A | C1A | C2A | C3A | 0.0 |
| N1A | C1A | C6 | O1 | −80.3(4) |
| C1A | N1A | C5A | C4A | 0.0 |
| CIA | C2A | C3A | C4A | 0.0 |
| C2A | C1A | C6 | O1 | 98.2(4) |
| C2A | C3A | C4A | F1A | 178.1(5) |
| C2A | C3A | C4A | C5A | 0.0 |
| C3A | C4A | C5A | N1A | 0.0 |
| C5A | N1A | C1A | C2A | 0.0 |
| C5A | N1A | CIA | C6 | 178.6(5) |
| N1B | C1B | C2B | F2B | −171.8(10) |
| N1B | C1B | C2B | C3B | 0.0 |
| N1B | C1B | C6 | O1 | −78.4(6) |
| C1B | N1B | C5B | C4B | 0.0 |
| C1B | C2B | C3B | C4B | 0.0 |
| C2B | C1B | C6 | O1 | 107.1(7) |
| C2B | C3B | C4B | F1B | 177.7(10) |
| C2B | C3B | C4B | C5B | 0.0 |
| C3B | C4B | C5B | N1B | 0.0 |
| C5B | N1B | C1B | C2B | 0.0 |
| C5B | N1B | C1B | C6 | −174.8(10) |
| N2 | C10 | C11 | C11 | −178.94(17) |
| N2 | C10 | C11 | C7 | 0.3(3) |
| N2 | C13 | C14 | C15 | 178.2(2) |
| N2 | C13 | C14 | C18 | −1.4(4) |
| N2 | C13 | C17 | F3 | −2.4(4) |
| N2 | C13 | C17 | C16 | −179.0(2) |
| N3 | C16 | C17 | F3 | −175.2(3) |
| N3 | C16 | C17 | C13 | 1.3(4) |
| N4 | N5 | C21 | C20 | −0.2(4) |
| N4 | N5 | C21 | C22A | 176.3(4) |
| N4 | N5 | C21 | C22B | −160.4(8) |
| N4 | C16 | C17 | F3 | 2.2(4) |
| N4 | C16 | C17 | C13 | 178.7(3) |
| N4 | C19 | C20 | C21 | −0.6(3) |
| N5 | N4 | C16 | N3 | −150.1(3) |
| N5 | N4 | C16 | C17 | 32.3(4) |
| N5 | N4 | C19 | C20 | 0.5(4) |
| N5 | C21 | C22A | O3A | −123.6(4) |
| N5 | C21 | C22A | C23A | −7.3(7) |
| N5 | C21 | C22A | C24A | 116.0(5) |
| N5 | C21 | C22B | O3B | 52.1(16) |
| N5 | C21 | C22B | C23B | −69.3(13) |
| N5 | C21 | C22B | C24B | 178.0(10) |
| C6 | O1 | C7 | C8 | −5.3(4) |
| C6 | O1 | C7 | C11 | 175.3(3) |
| C6 | C1A | C2A | F2A | 1.8(5) |
| C6 | C1A | C2A | C3A | −178.5(5) |
| C6 | C1B | C2B | F2B | 2.4(10) |
| C6 | C1B | C2B | C3B | 174.3(11) |
| C7 | O1 | C6 | C1A | −163.6(3) |
| C7 | O1 | C6 | C1B | −174.0(4) |
| C7 | C8 | C9 | N2 | 2.2(4) |
| C7 | C8 | C9 | C12 | −179.7(3) |
| C8 | C7 | C11 | C11 | −177.1(2) |
| C8 | C7 | C11 | C10 | 3.7(4) |
| C9 | N2 | C10 | O2 | 177.9(2) |
| C9 | N2 | C10 | C11 | −3.3(3) |
| C9 | N2 | C13 | C14 | −108.7(3) |
| C9 | N2 | C13 | C17 | 69.5(3) |
| C10 | N2 | C9 | C8 | 2.1(4) |
| C10 | N2 | C9 | C12 | −176.0(2) |
| C10 | N2 | C13 | C14 | 77.3(3) |
| C10 | N2 | C13 | C17 | −104.6(3) |
| C11 | C7 | C8 | C9 | −5.0(4) |
| C13 | N2 | C9 | C8 | −171.5(2) |
| C13 | N2 | C9 | C12 | 10.4(4) |
| C13 | N2 | C10 | O2 | −8.3(3) |
| C13 | N2 | C10 | C11 | 170.5(2) |
| C13 | C14 | C15 | N3 | 0.2(5) |
| C14 | C13 | C17 | F3 | 175.8(3) |
| C14 | C13 | C17 | C16 | −0.9(4) |
| C15 | N3 | C16 | N4 | −178.5(3) |
| C15 | N3 | C16 | C17 | −0.9(4) |
| C16 | N3 | C15 | C14 | 0.2(5) |
| C16 | N4 | N5 | C21 | −177.8(3) |
| C16 | N4 | C19 | C20 | 178.0(3) |
| C17 | C13 | C14 | C15 | 0.1(4) |
| C17 | C13 | C14 | C18 | −179.5(3) |
| C18 | C14 | C15 | N3 | 179.8(3) |
| C19 | N4 | N5 | C21 | −0.2(4) |
| C19 | N4 | C16 | N3 | 32.6(4) |
| C19 | N4 | C16 | C17 | −144.9(3) |
| C19 | C20 | C21 | N5 | 0.5(4) |
| C19 | C20 | C21 | C22A | −175.7(4) |
| C19 | C20 | C21 | C22B | 157.5(9) |
| C20 | C21 | C22A | O3A | 52.2(6) |
| C20 | C21 | C22A | C23A | 168.6(5) |
| C20 | C21 | C22A | C24A | −68.2(6) |
| C20 | C21 | C22B | O3B | −103.8(14) |
| C20 | C21 | C22B | C23B | 134.9(10) |
| C20 | C21 | C22B | C24B | 22.2(17) |

TABLE 11h
Hydrogen Atom Coordinates (Å × 10⁴) and Isotropic Displacement
Parameters (Å² × 10³) for Compound 1

| Atom | x | y | z | U(eq) |
|---|---|---|---|---|
| H3C | 11720 | 3177 | 5943 | 101 |
| H3D | 10548 | 943 | 4460 | 101 |
| H3A | 1580 | 5187 | 5510 | 61 |
| H5A | 148 | 5816 | 3077 | 62 |
| H3B | 1030 | 5419 | 5107 | 61 |
| H5B | 247 | 5965 | 2440 | 62 |
| H6AA | 3690 | 6265 | 2556 | 44 |
| H6AB | 4290 | 5850 | 3436 | 44 |
| H6BC | 3803 | 6281 | 2581 | 44 |
| H6BD | 4201 | 5800 | 3521 | 44 |
| H8 | 5590 | 5932 | 2493 | 33 |
| H12A | 8065 | 5601 | 2602 | 52 |
| H12B | 7365 | 6409 | 2125 | 52 |
| H12C | 8062 | 5706 | 1513 | 52 |
| H15 | 9789 | 3321 | −30 | 40 |
| H18A | 7337 | 3329 | −399 | 54 |
| H18B | 8252 | 3997 | −782 | 54 |
| H18C | 7341 | 4451 | −173 | 54 |
| H19 | 11853 | 3559 | 2491 | 40 |
| H20 | 12400 | 3098 | 4088 | 47 |
| H23A | 9431 | 2252 | 6269 | 93 |
| H23B | 9186 | 1787 | 5293 | 93 |
| H23C | 9112 | 2923 | 5427 | 93 |
| H23D | 9177 | 2475 | 5469 | 93 |
| H23E | 9964 | 3363 | 5527 | 93 |
| H23F | 9979 | 2555 | 6303 | 93 |
| H24A | 11998 | 1596 | 5351 | 91 |
| H24B | 10984 | 1003 | 5055 | 91 |
| H24C | 11168 | 1268 | 6106 | 91 |
| H24D | 11646 | 1673 | 6169 | 91 |
| H24E | 11834 | 2768 | 5891 | 91 |
| H24F | 12283 | 1927 | 5260 | 91 |
| H4C | 13240(60) | 4020(40) | 5730(50) | 89 |
| H4D | 13720(40) | 3020(40) | 6040(50) | 89 |

TABLE 11i
Atomic Occupancy for Compound 1

| Atom | Occupancy |
|---|---|
| F1A | 0.653(4) |
| F2B | 0.347(4) |
| O3B | 0.241(4) |
| C1A | 0.653(4) |
| H3A | 0.653(4) |
| H5A | 0.653(4) |
| C2B | 0.347(4) |
| C4B | 0.347(4) |
| H6AA | 0.653(4) |
| H6BD | 0.347(4) |
| C23A | 0.759(4) |
| H23C | 0.759(4) |
| H23E | 0.241(4) |
| H24A | 0.759(4) |
| C24B | 0.241(4) |
| H24F | 0.241(4) |
| H4D | 0.759(4) |
| F1B | 0.347(4) |
| O3A | 0.759(4) |
| H3D | 0.241(4) |
| C2A | 0.653(4) |
| C4A | 0.653(4) |
| N1B | 0.347(4) |
| C3B | 0.347(4) |
| C5B | 0.347(4) |
| H6AB | 0.653(4) |
| C22A | 0.759(4) |
| H23A | 0.759(4) |
| C23B | 0.241(4) |
| H23F | 0.241(4) |
| H24B | 0.759(4) |
| H24D | 0.241(4) |
| O4A | 0.759(4) |
| F2A | 0.653(4) |
| H3C | 0.759(4) |
| N1A | 0.653(4) |
| C3A | 0.653(4) |
| C5A | 0.653(4) |
| C1B | 0.347(4) |
| H3B | 0.347(4) |
| H5B | 0.347(4) |
| H6BC | 0.347(4) |
| C22B | 0.241(4) |
| H23B | 0.759(4) |
| H23D | 0.241(4) |
| C24A | 0.759(4) |
| H24C | 0.759(4) |
| H24E | 0.241(4) |
| H4C | 0.759(4) |

### Example 2—Synthesis of Amorphous Compound 1

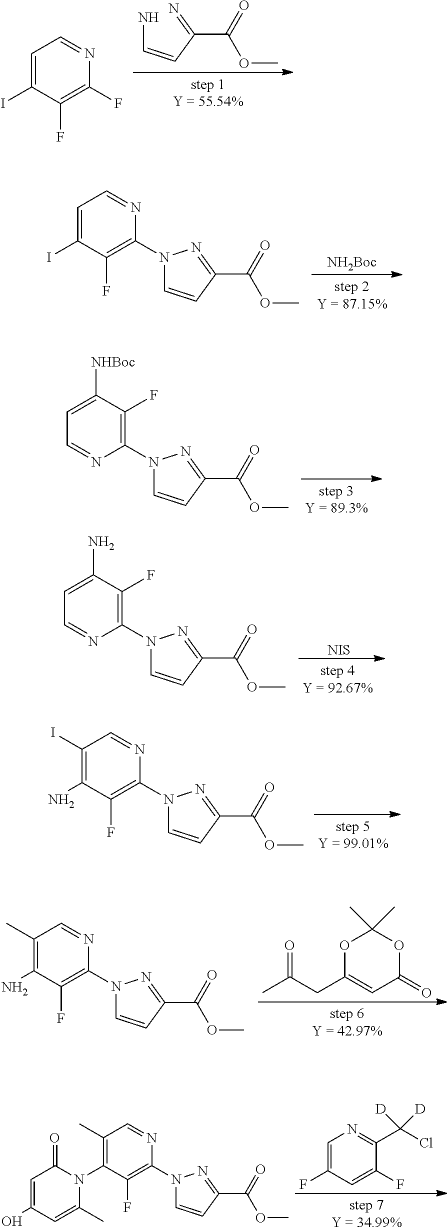

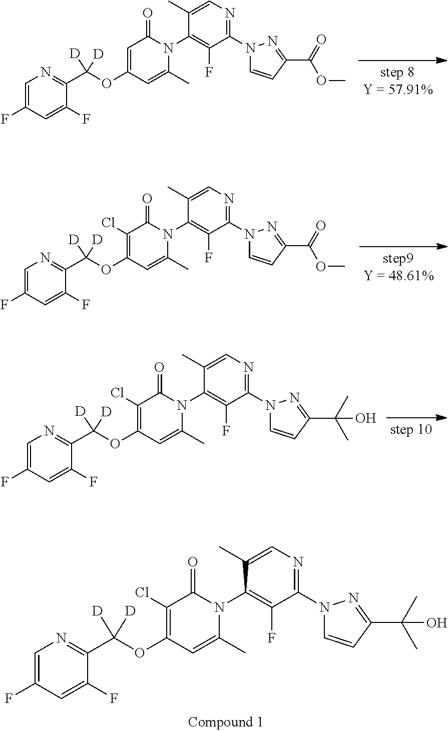

Step 1: Preparation of methyl 1-(3-fluoro-4-iodopyridin-2-yl)-1H-pyrazole-3-carboxylate:

A mixture of 2,3-difluoro-4-iodopyridine (50.00 g, 207.49 mmol, 1.00 equiv), methyl 1H-pyrazole-3-carboxylate (23.53 g, 186.74 mmol, 0.90 equiv) and Cs₂CO₃ (67.60 g, 207.49 mmol, 1.00 equiv) in DMF (500 mL) was stirred for 2 h at 100° C. under nitrogen atmosphere. The reaction was monitored by LCMS. The mixture was allowed to cool down to room temperature. The resulting mixture was filtered, the filter cake was washed with EtOAc (3×300 mL). The filtrate was concentrated under reduced pressure. The residue was purified by trituration with water (1000 mL). The precipitated solids were collected by filtration and washed with Et2O (3×100 mL). This resulted in methyl 1-(3-fluoro-4-iodopyridin-2-yl)-1H-pyrazole-3-carboxylate (40.00 g, 55.54%) as a white solid. LC-MS: (ES+H, m/z): [M+H]⁺=348.0. ¹H NMR (300 MHz, DMSO-d₆) δ8.51 (d, J=2.7, 1H), 8.13-8.00 (m, 2H), 7.03 (d, J=2.7 Hz, 1H), 3.87 (s, 3H).

Step 2: Preparation of methyl 1-(4-((tert-butoxycarbonyl)amino)-3-fluoropyridin-2-yl)-1H-pyrazole-3-carboxylate:

To a stirred mixture of methyl 1-(3-fluoro-4-iodopyridin-2-yl)-1H-pyrazole-3-carboxylate (50.00 g, 144.06 mmol, 1.00 equiv) and tert-butyl carbamate (33.75 g, 288.12 mmol, 2.00 equiv) in dioxane (200 mL) were added CsF (65.65 g, 432.18 mmol, 3.00 equiv), XantPhos (8.33 g, 14.41 mmol, 0.10 equiv) and Pd₂(dba)₃ (6.59 g, 7.20 mmol, 0.05 equiv) at room temperature under nitrogen atmosphere. The resulting mixture was stirred for 2 h at 100° C. under nitrogen atmosphere. The reaction was monitored by LCMS. The mixture was allowed to cool down to room temperature. The resulting mixture was filtered, the filter cake was washed with EtOAc (3×400 mL). The filtrate was concentrated under reduced pressure. The residue was purified by silica gel column chromatography, eluted with PE/EA (10:1-3:1) to afford methyl 1-(4-((tert-butoxycarbonyl)amino)-3-fluoropyridin-2-yl)-1H-pyrazole-3-carboxylate (42.2 g, 87.15%) as a yellow solid. LC-MS: (ES+H, m/z): [M+H]⁺=337.15.

Step 3: Preparation of methyl 1-(4-amino-3-fluoropyridin-2-yl)-1H-pyrazole-3-carboxylate:

A solution of methyl 1-(4-((tert-butoxycarbonyl)amino)-3-fluoropyridin-2-yl)-1H-pyrazole-3-carboxylate (50 g, 148.67 mmol, 1.00 equiv) in DCM (500 mL) was treated with TFA (250 mL) for 1 h at room temperature under nitrogen atmosphere. The reaction was monitored by LCMS. The resulting mixture was concentrated under reduced pressure. The resulting mixture was diluted with DCM (250 mL). The mixture was basified to pH 9 with saturated NaHCO₃ (aq.). The resulting mixture was extracted with CH₂Cl₂ (3×250 mL). The combined organic layers were washed with brine (1×1000 mL), dried over anhydrous Na₂SO₄. After filtration, the filtrate was concentrated under reduced pressure, to afford methyl 1-(4-amino-3-fluoropyridin-2-yl)-1H-pyrazole-3-carboxylate (31.36 g, 89.30%) as a white solid. LC-MS: (ES+H, m/z): [M+H]⁺=237.1

Step 4: Preparation of methyl 1-(4-amino-3-fluoro-5-iodopyridin-2-yl)-1H-pyrazole-3-carboxylate:

A solution of methyl 1-(4-amino-3-fluoropyridin-2-yl)-1H-pyrazole-3-carboxylate (40.00 g, 169.34 mmol, 1.00 equiv), NIS (45.70 g, 203.21 mmol, 1.20 equiv) and TsOH·H₂O (1.61 g, 8.47 mmol, 0.05 equiv) in MeCN (250 mL) was stirred for 2 h at 60° C. under nitrogen atmosphere. The reaction was monitored by LCMS. The mixture was allowed to cool down to room temperature. The resulting mixture was diluted with ethyl acetate (500 mL). The combined organic layers were washed with brine (3×500 mL), dried over anhydrous Na₂SO₄ to afford methyl 1-(4-amino-3-fluoro-5-iodopyridin-2-yl)-1H-pyrazole-3-carboxylate (58.7 g, 92.67%) as a yellow solid. LC-MS: (ES+H, m/z): [M+H]⁺=362.90. ¹H NMR (300 MHz, DMSO-d₆) δ8.40 (d, J=2.6 Hz, 1H), 8.24 (s, 1H), 6.99 (d, J=2.6 Hz, 1H), 6.78 (s, 2H), 3.86 (s, 3H).

Step 5: Preparation of methyl 1-(4-amino-3-fluoro-5-methylpyridin-2-yl)-1H-pyrazole-3-carboxylate:

A mixture of methyl 1-(4-amino-3-fluoro-5-iodopyridin-2-yl)-1H-pyrazole-3-carboxylate (25.00 g, 69.04 mmol, 1.00 equiv), Pd(dppf)Cl2 (5.01 g, 6.90 mmol, 0.10 equiv), Cs₂CO₃ (67.49 g, 207.12 mmol, 3.00 equiv) and trimethyl-1,3,5,2,4,6-trioxatriborinane (87.05 g, 345.20 mmol, 5.00 equiv, 50 wt %) in dioxane (400 mL) was stirred for 2 h at 100° C. under nitrogen atmosphere. The reaction was monitored by LCMS. The mixture was allowed to cool down to room temperature. The resulting mixture was filtered, the filter cake was washed with ethyl acetate (3×1000 mL). The filtrate was concentrated under reduced pressure. The residue was purified by silica gel column chromatography, eluted with PE/EA (3:1-1:1) to afford methyl 1-(4-amino-3-fluoro-5-methylpyridin-2-yl)-1H-pyrazole-3-carboxylate (17.10 g, 99.01%) as a light-yellow solid. LC-MS: (ES+H, m/z): [M+H]⁺=251.2.

Step 6: Preparation of methyl 1-(3′-fluoro-4-hydroxy-5′,6-dimethyl-2-oxo-2H-[1,4′-bipyridin]-2′-yl)-1H-pyrazole-3-carboxylate:

To a solution of methyl 1-(4-amino-3-fluoro-5-methylpyridin-2-yl)-1H-pyrazole-3-carboxylate (25.00 g, 99.91 mmol, 1.00 equiv) and 2,2-dimethyl-6-(2-oxopropyl)-1,3-dioxin-4-one (36.78 g, 199.82 mmol, 2.00 equiv) in dioxane (260 mL) was added Ti(Oi-Pr)₄ (2.84 g, 9.99 mmol, 0.10 equiv), the resulting mixture was stirred for 1 h at 90° C. under nitrogen atmosphere. The reaction was monitored by LCMS. The mixture was allowed to cool down to room temperature. The mixture was followed by the addition of H₂SO₄ (9.79 g, 99.91 mmol, 1.00 equiv) dropwise at room temperature. The resulting mixture was stirred for 1 h at 90° C. under nitrogen atmosphere. The reaction was monitored by LCMS. The mixture was allowed to cool down to room temperature. The resulting mixture was concentrated under reduced pressure. The residue was purified by trituration with water (200 mL) and Et₂O (100 mL). The precipitated solids were collected by filtration and washed with Et₂O (3×100 mL), to afford methyl 1-(3′-fluoro-4-hydroxy-5′,6-dimethyl-2-oxo-2H-[1,4′-bipyridin]-2′-yl)-1H-pyrazole-3-carboxylate (15.38 g, 42.97%) as a brown solid. LC-MS: (ES+H, m/z): [M+H]⁺=359.0.

Step 7: Preparation of methyl 1-(4-((3,5-difluoropyridin-2-yl)methoxy-d2)-3′-fluoro-5′,6-dimethyl-2-oxo-2H-[1,4′-bipyridin]-2′-yl)-1H-pyrazole-3-carboxylate:

To a stirred mixture of methyl 1-(3′-fluoro-4-hydroxy-5′,6-dimethyl-2-oxo-2H-[1,4′-bipyridin]-2′-y1)-1H-pyrazole-3-carboxylate (10.00 g, 42.51 mmol, 1.00 equiv) and 2-(chloromethyl-d2)-3,5-difluoropyridine (10.52 g, 63.77 mmol, 1.50 equiv) in DMF (100 mL) were added Cs₂CO₃ (41.56 g, 127.53 mmol, 3.00 equiv) and 18-Crown-6 (1.12 g, 4.25 mmol, 0.10 equiv) at room temperature under nitrogen atmosphere. The resulting mixture was stirred for 2 h at 70° C. under nitrogen atmosphere. The reaction was monitored by LCMS. The mixture was allowed to cool down to room temperature. The resulting mixture was diluted with EtOAc (500 mL). The organic layers were washed with water (5×500 mL), dried over anhydrous Na₂SO₄. After filtration, the filtrate was concentrated under reduced pressure. The residue was purified by silica gel column chromatography, eluted with PE/EA (1:1-1:2), to afford methyl 1-(4-((3,5-difluoropyridin-2-yl)methoxy-d2)-3′-fluoro-5′,6-dimethyl-2-oxo-2H-[1,4′-bipyridin]-2′-y1)-1H-pyrazole-3- carboxylate (7.25 g, 34.99%) as a white solid. LC-MS: (ES+H, m/z): [M+H]⁺=488.15.

Step 8: Preparation of methyl 1-(3-chloro-4-((3,5-difluoropyridin-2-yl)methoxy-d2)-3′-fluoro-5′,6-dimethyl-2-oxo-2H-[1,4′-bipyridin]-2′-yl)-1H-pyrazole-3-carboxylate:

A mixture of methyl 1-(4-((3,5-difluoropyridin-2-yl)methoxy-d₂)-3′-fluoro-5′,6-dimethyl-2-oxo-2H-[1,4′-bipyridin]-2′-yl)-1H-pyrazole-3-carboxylate (10.00 g, 20.52 mmol, 1.00 equiv) , NCS (3.56 g, 26.68 mmol, 1.30 equiv) and 2,2-dichloroacetic acid (0.26 g, 2.05 mmol, 0.10 equiv) in i-PrOH (100 mL) was stirred for 1 h at 60° C. under nitrogen atmosphere. The reaction was monitored by LCMS. The mixture was allowed to cool down to room temperature. The resulting mixture was diluted with EtOAc (200 mL). The resulting mixture was washed with 3×200 mL of water. The resulting mixture was concentrated under reduced pressure. The residue was purified by silica gel column chromatography, eluted with PE/EA (1:1) to afford methyl 1-(3-chloro-4-((3,5-difluoropyridin-2-yl)methoxy-d₂)-3′-fluoro-5′,6-dimethyl-2-oxo-2H-[1,4′-bipyridin]-2′-yl)-1H-pyrazole-3- carboxylate (6.20g, 57.91%) as a white solid. LC-MS: (ES+H, m/z): [M+H]⁺=522.2.

Step 9: Preparation of 3-chloro-4-((3,5-difluoropyridin-2-yl)methoxy-d₂)-3′-fluoro-2′-(3-(2-hydroxypropan-2-yl)-1H-pyrazol-1-yl)-5′,6-dimethyl-2H-[1,4′-bipyridin]-2-one:

To a stirred solution of methyl 1-(3-chloro-4-((3,5-difluoropyridin-2-yl)methoxy-d₂)-3′-fluoro-5′,6-dimethyl-2-oxo-2H-[1,4′-bipyridin]-2′-yl)-1H-pyrazole-3-carboxylate (5.00 g, 9.58 mmol, 1.00 equiv) in THF (50 mL) was added CH₃MgBr (31.93 mL, 95.80 mmol, 10.00 equiv (3M in THF)) dropwise at 0° C. under nitrogen atmosphere. The resulting mixture was stirred for 2 h at 0° C. under nitrogen atmosphere. Desired product could be detected by LCMS. The mixture was allowed to cool down to 0° C. The reaction was quenched by the addition of sat. NH₄Cl (aq.) (150 mL) at 0° C. The resulting mixture was extracted with EtOAc (4×300 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:1-1:3), the filtrate was concentrated under reduced pressure to afford 3-chloro-4-((3,5-difluoropyridin-2-yl)methoxy-d₂)-3′-fluoro-2′-(3-(2-hydroxypropan-2-yl)-1H-pyrazol-1-yl)-5′,6-dimethyl-2H-[1,4′-bipyridin]-2-one (2.43 g, 48.61%) as a white solid. LC-MS: (ES+H, m/z): [M+H]⁺=522.1.

Step 10: Preparation of (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1):

The rac-mixture (17.50 g) was separated by Prep-Chiral SFC with the following conditions (Column: NB_CHIRALPAK AD-H, 5*25 cm, 5 μm; Mobile Phase A: CO₂, Mobile Phase B: MeOH--HPLC; Flow rate: 180 mL/min; Gradient: isocratic 35% B; Column Temperature (° C.): 35; Back Pressure(bar): 100; Wave Length: 210 nm; RT1(min): 4.01; RT2(min): 5.36; Sample Solvent: MeOH: DCM=1:1--HPLC; Injection Volume: 2 mL; Number Of Runs: 42) to afford compound 1 (6.49 g, ee=100%). LC-MS: (ES+H, m/z): [M+H]⁺=522.15. ¹H NMR (400 MHz, DMSO-d₆) δ8.61 (d, J=2.3 Hz, 1H), 8.50 (s, 1H), 8.37 (d, J=2.6 Hz, 1H), 8.15-8.06 (m, 1H), 6.91 (s, 1H), 6.60 (d, J=2.6 Hz, 1H), 5.13 (s, 1H), 2.09 (d, J=16.1 Hz, 6H), 1.48 (s, 6H). ¹⁹NMR (377 MHz, DMSO) δ−120.25, −120.27, −122.29, −122.31, −137.97.

### Example 3: Polymorph Screening

Polymorph screening of compound 1 was carried out using commonly used solvents and various crystallization methods, including anti-solvent precipitation, slurry conversion, cooling, evaporation, and solution vapor diffusion. A total of seven crystal forms (Forms I-IV, VI-VIII) were identified, and two new crystal forms (Forms IX and X) appeared in follow-up slurry competition experiments. Additional polymorph screening by slurry conversion was carried out using Form X as starting material, and the results showed no new crystal forms were found. The characterization data of Compound 1 polymorphs are given in Table 12. Among them, there are three hydrates (Forms I, IV, IX) and two anhydrous (Forms VIII, X). Form X is the more stable anhydrous form with the higher melting point and enthalpy.

TABLE 12
Characterization Data of Compound 1 Polymorphs

| | | DSC Endo | | |
|---|---|---|---|---|
| | Crystal- | Onset/Peak T | TGA | ¹H-NMR |
| Form | linity | (° C.), | Wt. Loss % | Solvent |
| Solvation | by XRPD | ΔH (J/g) | @T (° C.) | Residue |
| Form I | High | 27/62, 48 | 2.6, RT-90 | Negligible |
| Hydrate | | 98/104, 13 | | solvent |
| | | | | residue |
| Form II | High | 103/116, 70 | 1.0, 90-130 | 2.1% EtOH, |
| Isostructural | | 148/155, 19 | | 0.3% CYH |
| solvate | | | | |
| Form III | High | 101/101, 94 | 12.8, 70-140 | 14.4% MTBE |
| Isostructural | | | | |
| solvate | | | | |
| Form IV | High | 36/52, 47 | 1.4, RT-100 | Negligible |
| Hydrate | | 104/110, 15 | | solvent |
| | | | | residue |
| Form VI | High | 100/105, 46 | 5.0, 65-100 | 4.9% MIBK |
| Isostructural | | | | |
| solvate | | | | |
| Form VII | High | 85/99, 106 | 9.0, 75-120 | 12.8% MTBE |
| Isostructural | | 148/154, 14 | 2.9, 123-165 | |
| solvate | | | | |
| Form VIII | High | 48/50, 2 | 0.2, RT-50 | Negligible |
| anhydrous | | 15/155, 42 | | solvent |
| | | | | residue |
| Form IX | High | 29/56, 8 | 1.3, RT-80 | Negligible |
| Hydrate | | 114/118, 47 | | solvent |
| | | | | residue |
| Form X | High | 157/158, 71 | ~0, RT-150 | Negligible |
| anhydrous | | | | solvent |
| | | | | residue |

Form V was Form I with less diffraction peaks in XRPD pattern.

Slurry competition experiments of anhydrous and hydrates were performed to establish stability relationship. The results showed Form X is thermodynamically more stable even in pure water at room temperature. Form X was further evaluated, and its solid-state properties are presented in Table 13. Form X was non-hygroscopic with water uptake of 0.11/0.16% at 80/90% RH. Solid-state stability results showed that Form X was physically and chemically stable at 40° C./75% RH (open) and 60° C. (capped) for one week.

Overall, the results from polymorph screen of compound 1 indicated this compound has a complex polymorphism landscape. Freebase Form X is a stable anhydrous form with acceptable solid-state properties.

TABLE 13
Solid-state Properties of Compound 1 Form X

| Solid Form | | Form X |
|---|---|---|
| Solvation | | Anhydrous |
| Microscopy | | Needle-like crystals with aggregation |
| Crystallinity | | High |
| DSC Endo | | 157/158, 71, melt |
| Onset/Peak T, ΔH | | |
| TGA Wt. Loss @T | | ~0 @RT-150° C. |
| DVS Wt. Gain % | | 0.11/0.16% at 80/90% RH, non-hygroscopic |
| XRPD after DVS | | No change |
| ¹H-NMR Solvent Residue | | Negligible solvent residue |
| Solubility ^{a} | SGF | 0.074/0.070/0.066 |
| (mg/mL) | FaSSIF | 0.069/0.070/0.074 |
| @0.5/2/24 h | FeSSIF | 0.097/0.097/0.124 |
| | Water | 0.074/0.074/0.070 |

| | |
|---|---|
| Stability | Physically stable at 40° C./75% RH (open) |
| | and 60° C. for 1 week; no form change |
| | with slight crystallinity decrease after |
| | manual grinding |
| Comments | Form X has the highest melting point |
| | and enthalpy |

^{a} Form X remained unchanged during solubility test in all media

### Solubility Estimation

The solvents used for solubility estimation and solid form screen are given in Table 14.

TABLE 14
List of Solvents

| Solvent | Solvent |
|---|---|
| Methanol (MeOH) | Isopropyl ether (IPE) |
| Ethanol (EtOH) | Anisole |
| Isopropyl Alcohol (IPA) | Cyclopentyl methyl ether (CPME) |
| Acetone | Acetonitrile (CAN) |
| Methyl ethyl ketone (MEK) | Water |
| Methyl isobutyl ketone (MIBK) | Dichloromethane (DCM) |
| Ethyl Acetate (EtOAc) | Toluene |
| Isopropyl Acetate (IPAc) | n-Heptane |
| Tetrahydrofuran (THF) | Cyclohexane (CYH) |
| 2-Methyltetrahydrofuran (2-MeTHF) | Methylcyclohexane (MeCYH) |
| Methyl tert-Butyl Ether (MTBE) | |

The solubility of compound 1 Form I and Form X was estimated at RT by visual observation in selected solvent systems. Approximately 5 mg solids were weighed into 8 mL glass vial, and then solvent was added stepwise until solids were dissolved completely or a total of solvent volume reached 5 mL. The results are summarized in Table 15. Form I showed high solubility (>100 mg/mL) in most tested solvents, and Form X showed decreased solubility in some solvents.

TABLE 15
Estimated Solubility of Compound 1 Forms I and X at RT

| Solubility | Solvent | |
|---|---|---|
| (mg/mL) | Form I | Form X |
| >100 | EtOH, Acetone, EtOAc, ACN, | ACN, Acetone, THF |
| | DCM, THF, | |
| | MeOH, IPA, 2-MeTHF, MIBK, | |
| | IPAc, Anisole, CPME | |
| 50~100 | N/A | 2-MeTHF |
| 20~50 | MTBE | MIBK, IPAc |
| 5~20 | N/A | EtOH |
| 1~5 | Toluene, IPE | MTBE, Toluene |
| <1 | Water, Heptane, CYH, IPE | N/A |

Values are reported as “<” if dissolution was not observed, and as “>” if dissolution occurred after addition of first aliquot.

### Characterization of Crystalline Freebase Forms

A total of nine crystalline forms were discovered, including three hydrates (Forms I, IV, IX) and two anhydrous forms (Forms VIII, X). The detailed characterization results of each form are presented below.

### Form I

Form I was first obtained via quench cooling in toluene and IPE/toluene or evaporation in MeOH/water.

### Synthesis of Compound 1 Form I

Amorphous compound 1 (50 mg) was stirred in water (0.5 mL) for 6 hrs at 25° C. The solid was collected by filtration and dried in oven at 40° C. overnight to provide Compound 1 Form I.

The sample was fine crystals under microscope. XRPD result (FIG. 2) confirmed the sample was highly crystalline. Thermal analysis showed 2.6% weight loss before 90° C., and two endothermic peaks at 27 and 98° C. (onset), due to dehydration and melting. Negligible solvent residue was detected by ¹H-NMR. Form I is a hydrate. After heating to 95° C., Form I remained unchanged indicating the heated sample absorbed moisture quickly after exposure to air.

TABLE 16
Characterization Data of Form I

| Item | Compound 1 Form I |
|---|---|
| PLM | Fine crystals |
| XRPD | High crystallinity |
| DSC Endo | 27/62° C., 48 J/g |
| Onset/Peak T, ΔH | 98/104, 13 J/g |
| TGA Wt. Loss @T | 2.6% @RT-90° C. |
| ¹H-NMR | Negligible solvent residue |

### Form II

Form II was obtained from Form I from EtOH and IPA systems. Two lots of Form II were characterized.

Form II (lot #1) was obtained via slurry of Form I in EtOH/CYH (1/4, v/v) at 50° C. for 7 days. Thermal analysis showed 1.0% weight loss at 90-130° C., and two endothermic peaks at 103 and 148° C. (onset), due to desolvation and melting. About 2.1% EtOH and 0.3% CYH were detected by ¹H-NMR. After heating to 130° C., Form II converted into Form VIII. This Form II lot is likely an EtOH solvate. Form II (Lot #2) was obtained in IPA with dissolution-precipitation process at RT. Thermal analysis showed 1.8% weight loss at 90-135° C., and two overlapped endothermic peaks at 99° C., due to desolvation. After heating to 145° C., amorphous was obtained. This Form II lot is likely an IPA solvate.

In some embodiments, Form II is an isostructural solvate which can contain different solvents in the same type of molecular network of host molecules.

### Form III

Form III was obtained from Form I in MTBE, IPE, 2-MeTHF, and THF systems. Two lots of Form III were characterized.

Form III (Lot #1) was obtained by slurry of Form I in 2-MeTHF/CYH (1/4, v/v) at RT for 7 days. Thermal analysis showed 9.2% weight loss at 50-135° C., and one broad endothermic peak at 78° C. (onset), due to desolvation. About 2.8% 2-MeTHF and 4.1% CYH were detected by ¹H-NMR. After heating to 120° C., Form III converted to amorphous. This lot of Form III might be a co-solvate of 2-MeTHF and CYH.

Form III (Lot #2) was obtained by slurry in MTBE at RT for 1 day. Thermal analysis showed 12.8% weight loss at 70-140° C., and overlapping endothermic peaks at 101° C. (onset), due to desolvation. About 14.4% MTBE was detected by ¹H-NMR. This lot of Form III was a MTBE solvate.

In some embodiments, Form III is an isostructural solvate which can contain different solvents in the same type of molecular network of host molecules.

### Form IV

Form IV was obtained by many conditions from Form I. For example, Form IV was obtained Form I by anti-solvent precipitation using in MeOH/water at RT, and chosen for characterization.

The sample was fine crystals with aggregation under microscope. Thermal analysis showed 1.4% weight loss before 100° C., and two endothermic peaks at 36 and 104° C. (onset), due to dehydration and melting. No obvious MeOH residue was detected by ¹H-NMR. Form IV is a hydrate.

### Form VI

Form VI was obtained from Form I from acetone, MIBK, EtOAc, and IPAc systems. Two lots of Form VI were characterized.

Form VI (Lot #1) was obtained from Form I by anti-solvent precipitation in EA/heptane at RT. Thermal analysis showed 1.0% weight loss at 70-120° C., and one broad endothermic peak at 97° C. (onset), due to desolvation. About 0.6% EtOAc and 4.3% heptane were detected by ¹H-NMR. After heating to 110° C., Form VI converted to amorphous. This lot Form VI might be a co-solvate of EtOAc and heptane.

Form VI (Lot #2) was obtained from Form I by anti-solvent addition in MIBK/CYH at RT. Thermal analysis showed 5.0% weight loss at 65-100° C., and one endothermic peaks at 100° C. (onset), due to desolvation. About 4.9% MIBK was detected by ¹H-NMR. This lot Form VI is a MIBK solvate.

In some embodiments, Form VI is an isostructural solvate which can contain different solvents in the same type of molecular network of host molecules.

### Form VII

Form VII was obtained Form I from acetone, MEK, 2-MeTHF, MTBE, and DCM systems. Two lots of Form VII were characterized.

Form VII (Lot #1) was obtained from Form I by slow evaporation in MTBE at RT. Thermal analysis showed 9.0% weight loss at 75-120° C. and 2.9% weight loss at 123-165° C., and two endothermic peaks at 85 and 148° C. (onset), due to desolvation and melting. About 12.8% MTBE were detected by ¹H-NMR. After heating to 110° C., amorphous with little Form VIII was obtained. This lot Form VII is a MTBE solvate.

Form VII (Lot #2) was obtained from Form I by anti-solvent precipitation in MEK/MeCYH at RT. Thermal analysis showed 5.2% weight loss at 60-100° C., and one endothermic peak at 76° C. (onset), due to desolvation. About 2.2% MEK and 3.9% MeCYH were detected by ¹H-NMR. This lot Form VII might be a co-solvate of MEK and MeCYH.

In some embodiments, Form VII is an isostructural solvate which can contain different solvents in the same type of molecular network of host molecules.

### Form VIII

Form VIII was obtained from Form I with non-aqueous solvent systems at 50 or 80° C. Form VIII was obtained from Form I by slurry in heptane at 80° C., and chosen for characterization. The sample was fine crystals with aggregation. Thermal analysis showed 0.2% weight loss before 50° C., and two endothermic peaks at 48 and 154° C. (onset), due to phase transition and melting. The phase transition signal around 50° C. was reversible. No obvious heptane residue was detected by ¹H-NMR. Form VIII is anhydrous.

### Form IX

Form IX appeared in slurry competition of Forms I and IV in MeOH/water at RT. Form IX was obtained by slurry in MeOH/water (1/4, v/v) with seed at RT, and chosen for characterization.

The sample was needle-like crystals under microscope. Thermal analysis showed 1.3% weight loss before 80° C., and two endothermic peaks at 29 and 114° C. (onset), due to dehydration and melting. No obvious MeOH residue was detected by ¹H-NMR. Form IX is a hydrate.

### Form X

### Original Synthesis of Compound 1 Form X

Compound 1 (3 mg each of Form IV and Form IX) was stirred in a mixture of water/methanol (19:1, 0.5 mL, pre-saturated with compound 1) for 3 days at room temperature. The solid was collected by filtration and dried in oven at 40° C. to provide compound 1 Form X.

### Synthesis of Compound 1 Form X (Alternative Procedure)

Amorphous compound 1 (275 g) was stirred in mixture of water/methanol (4:1, 4.1 L total) for 30 min at 20° C. Seed crystals of compound 1 Form X (1 g) was added and the mixture was stirred for another 16 hrs. The solid was collected by filtration, washed with water, and dried in oven at 40° C. to provide compound 1 Form X.

Form X was needle-like crystals with aggregation. Thermal analysis showed negligible weight loss before 150° C., and one sharp endothermic peak at 157° C. (onset), due to melting. No obvious MeOH residue was detected by ¹H-NMR. Form X is anhydrous.

### Example 4: Thermodynamic Stability Relationship Study

Slurry competition experiments were carried out between anhydrous and hydrates, to establish stability relationships. Appropriate amount of Form I was suspended in different solvents for pre-saturation at RT. Then the filtrate was added into equal amount of different forms, and the mixture was kept stirring at RT for certain time before XRPD tests. The results are summarized in Table 17. Form X is thermodynamically stable even in pure water.

TABLE 17
Results of Competitive Slurries at RT

| Input | Solvent (v/v) | Duration (d) | Output |
|---|---|---|---|
| Form VIII | Water | 1 | Form I |
| Form I + IV | Water | 3 | Form IV |
| | Water/MeOH (9/1) | 3 | Form IV + IX |
| | Water/MeOH (19/1) | 3 | Form IV + IX |
| Form IV + IX | Water | 3 | Form X + IV (trace) |
| | Water/MeOH (19/1) | 3 | Form X |
| | Water/MeOH (9/1) | 3 | Form X + IV |
| | Water/MeOH (17/3) | 3 | Form X |
| Form IV + X | Water | 2 | Form X |

### Evaluation of Form X

### Hygroscopicity

DVS was performed on Form X, to evaluate its hygroscopicity and physical stability under different humidity. DVS result showed Form X sample was non-hygroscopic with water uptake of 0.11/0.16% at 80/90% RH. The crystal form remained unchanged after DVS test.

### Solid-State Stability

Solid-state stability of Form X was conducted at 60° C. (capped) and 40° C./75% RH (open) for 7 days. The stability sample was dissolved in diluent to prepare solution at ˜1 mg/mL for HPLC purity analysis. Solid samples were analyzed by XRPD to check the crystal form. The results are summarized in Table 18. No form change or purity decrease was observed at both 60° C. (capped) and 40° C./75% RH (open) after 7 days, suggesting Form X was physically and chemically stable at tested stability conditions.

TABLE 18
Solid-state Stability Results of Form X

| Condition | Time | Purity (area %) | XRPD Result |
|---|---|---|---|
| Initial | / | 99.94 | Form X |
| 40° C./75% RH (open) | 1 week | 99.94/99.93 | Form X |
| 60° C. (capped) | 1 week | 99.93/99.94 | Form X |

### Mechanical Stability

Appropriate amount of Form X was manually ground by pestle and mortar for about 2 minutes and 5 minutes, and then analyzed by XRPD. The crystal form of Form X remained unchanged with slight crystallinity decrease after grinding, indicating it has acceptable mechanical stability.

### Solubility in Bio-Relevant Media

The solubility of Form X was measured in bio-relevant media (SGF, FaSSIF and FeSSIF) and water at 37° C. with 800 rpm for up to 24 hours. About 15 mg of Form X was weighed into sample vials and then 3 mL of three bio-relevant media and water were added to make suspensions, respectively. At 0.5, 2 and 24 hours, about 1 mL of each suspension was filtered, the filtrates were analyzed by HPLC and pH, and the filter cakes were analyzed by XRPD. Duplicate samples were prepared. The results are summarized in Table 19. Form X showed a solubility of 0.07˜0.12 mg/mL in FaSSIF, FeSSIF, SGF and water. A little higher solubility in FeSSIF was possibly due to the effect of bile salt. Form X remained unchanged after solubility test in all media.

TABLE 19
Data of Solubility Test in Bio-relevant Media

| | Solubility (mg/mL) | | | pH | XRPD |
|---|---|---|---|---|---|
| Media | 0.5 h | 2 h | 24 h | 0.5/2/24 h | 0.5/2/24 h |
| Water (pH 6.28) | 0.074 | 0.074 | 0.070 | 6.30/6.86/6.21 | Form X |
| FaSSIF (pH 6.43) | 0.069 | 0.070 | 0.074 | 6.51/6.51/6.48 | |
| FeSSIF (pH 4.94) | 0.097 | 0.097 | 0.124 | 4.89/4.92/4.98 | |
| SGF (pH 1.24) | 0.074 | 0.070 | 0.066 | 1.24/1.27/1.28 | |

### pH Solubility Profile

Different pH buffers (pH 1.0, 3.0, 5.0, 6.8 and 9.0) were prepared by method in Table 20. Then 10 mg of Form X was added into 2 mL of different buffers (pH=1.0, 3.0, 5.0, 6.8 and 9.0) to make suspensions. The suspensions were kept shaking with a speed of 1000 rpm at 25° C. for 4 and 24 hours. At each point, the suspensions were centrifuged, and the supernatant was inspected by HPLC/pH, and the wet cakes were analyzed by XRPD. Duplicate samples were prepared.

All the results are summarized in Table 21. pH solubility profile showed no pH-dependence and the solubility was 0.06˜0.08 mg/mL. Form X remained unchanged after pH solubility test.

TABLE 20
Preparation of Different pH Buffer

| pH Buffer | | Experimental Procedure |
|---|---|---|
| pH 1.0 | 0.1N HCl | / |
| pH 3.0 | 25 mM citrate, | Anhydrous citric acid (0.480 g), NaOH |
| | I = 0.1M | (0.2M, 5.856 mL) and NaCl (0.526 g) |
| | | was dissolved in water, the total |
| | | volume of water is 100 mL. |
| pH 5.0 | 25 mM citrate, | Anhydrous citric acid (0.480 g), NaOH |
| | I = 0.1M | (0.2M, 23.818 mL) and NaCl (0.178 g) |
| | | was dissolved in water, the total |
| | | volume of water is 100 mL. |
| pH 6.8 | 25 mM Phosphate, | Monohydrate NaH₂PO₄ (0.345 g), NaOH |
| | I = 0.1M | (0.2M, 5.904 mL) and NaCl (0.335 g) |
| | | was dissolved in water, the total |
| | | volume of water is 100 mL. |
| pH 9.0 | 25 mM DEA, | Diethanolamine (0.2629 g), HCl (0.1M, |
| | I = 0.1M | 12.375 mL) and NaCl (0.580 g) was |
| | | dissolved in water, the total volume |
| | | of water is 100 mL. |

TABLE 21
pH Solubility Results of Form X

| | | | | Solubility | | |
|---|---|---|---|---|---|---|
| | pH of Filtrate | | | (1T/2T, mg/mL) | | XRPD |
| pH | Initial | 4 h | 24 h | 4 h | 24 h | 4/24 h |
| pH 1.0 | 1.04 | 1.28 | 1.21 | 0.074 | 0.075 | Unchanged |
| pH 3.0 buffer | 3.02 | 3.04 | 3.11 | 0.060 | 0.073 | |
| pH 5.0 buffer | 4.96 | 4.88 | 4.98 | 0.061 | 0.060 | |
| pH 6.8 buffer | 6.93 | 6.77 | 6.79 | 0.060 | 0.060 | |
| pH 9.0 buffer | 9.08 | 8.87 | 9.03 | 0.060 | 0.062 | |

### Example 5: Slurry Conversion

### Form I as Starting Material

Appropriate amount of Form I was added into different solvents to make suspensions, which were kept stirring at RT and 50° C. for 3 and 7 days, and at 80° C. for 3 days. Solid samples were collected by centrifugation and analyzed by XRPD. The results are summarized in Table 22 to Table 24. Forms I-IV, VII and VIII were obtained by slurry experiments of Form I.

TABLE 22
Results of Slurry Conversion of Form I at RT

| | XRPD Result | |
|---|---|---|
| Solvent (v/v) | Day 3 | Day 7 |
| Heptane | Form I | Form I |
| MeCYH | Form I | Form I |
| CYH | Form I | Form I |
| MTBE | Form III | Form III |
| IPE | Similar to Form III | Similar to Form III |
| Water | Form I | Form I |
| IPE/water (200/1) | Form IV | Form IV |
| Acetone/water (1/4) | Form VII | Form VII |
| ACN/water (1/9) | Form IV | Form IV |
| Toluene/heptane (1/4) | Form IV | Form IV |
| IPAc/heptane (1/4) | Form IV | Form IV |
| 2-MeTHF/CYH (1/4) | Similar to Form III | Similar to Form III |
| EtOH/CYH (1/4) | Similar to Form II | Similar to Form II |
| Toluene | Gel-like on the wall | Form IV (6 d) |
| | | Gel-like on the wall |

TABLE 23
Results of Slurry Conversion of Form I at 50° C.

XRPD Result

| Solvent (v/v) | Day 3 | Day 7 |
|---|---|---|
| Heptane | Form IV | Form IV |
| MeCYH | Form IV | Form IV |
| CYH | Form IV | Form IV |
| MTBE | Form III | Form III |
| IPE | Form IV | Form IV + VIII |
| Water | Form IV | Form IV |
| IPE/water (200/1) | Form IV | Form IV |
| Acetone/water (1/4) | Form IV | Form IV |
| ACN/water (1/9) | Form IV | Form IV |
| Toluene/heptane (1/4) | Form IV | Form IV |
| IPAc/heptane (1/4) | Form IV + VIII | Form IV + VIII |
| 2-MeTHF/CYH (1/4) | Form VII | Form III |
| EtOH/CYH (1/4) | Similar to Form II | Similar to Form II |
| Toluene | Clear | Clear |

TABLE 24
Results of Slurry Conversion of Form I at 80° C.

| Solvent | XRPD Result-Day 3 |
|---|---|
| Water | Form IV |
| Heptane | Form VIII |
| MeCYH | Form VIII |
| CYH | Form VIII |

### Form X as Starting Material

Appropriate amount of Form X was added into different solvents to make suspensions, which were kept stirring at RT and 50° C. for 3 and 7 days, and at 80° C. for 3 days. Solid samples were collected by centrifugation and analyzed by XRPD. Forms III and X were obtained were obtained by slurry experiments of Form X. The results are summarized in Table 25 to Table 27.

TABLE 25
Results of Slurry Conversion of Form X at RT

XRPD Result

| Solvent (v/v) | Day 3 | Day 7 |
|---|---|---|
| MTBE | Form III | Form III |
| IPE | Form X | Form X |
| Toluene | Form X | Form X |
| IPAc | Form X | Form X |
| EtOH | Clear | Clear |
| IPA | Form X | Form X |
| Water | Form X | Form X |
| MIBK | Form X | Form X |
| MeOH/water (1/4) | Form X | Form X |
| Acetone/water (1/3) | Form X | Form X |

TABLE 26
Results of Slurry Conversion of Form X at 50° C.

XRPD Result

| Solvent (v/v) | Day 3 | Day 7 |
|---|---|---|
| MTBE | Form X | Form III |
| IPE | Form X | Form X |
| Toluene | Form X | Form X |
| IPA | Form X | Form X |
| Water | Form X | Form X |
| MeOH/Water (1/4) | Form X | Form X |
| Acetone/water (1/3) | Form X | Form X |

TABLE 27
Results of Slurry Conversion of Form X at 80° C.

| Solvent (v/v) | XRPD Result-Day 3 |
|---|---|
| CYH | Form X |
| MeCYH | Form X |
| Heptane | Form X |
| Water | Form X |
| IPA/Water (1/3) | Form X |
| IPA/Water (1/2) | Clear |
| IPAc/Heptane (1/2) | Form X |

### Example 6: Evaporation

1. Evaporation was performed in 14 selected solvents according to the solubility data. About 15 mg of starting material was dissolved in selected solvents to get a clear solution. Then the filtrate in a clean vial was covered with pin-hole film or foil and placed at RT for slow evaporation until solid precipitation. The results are summarized in Table 28. Forms I and VII were obtained in evaporation experiments, and a new pattern was observed in IPE.

DSC result showed the new pattern sample had one broad endothermic peak at 69° C., possibly due to desolvation. No further characterization was performed due to limited amount.

TABLE 28
Results of Slow Evaporation

| Solvent (v/v) | XRPD Result |
|---|---|
| MTBE | Similar to Form VII |
| MTBE/toluene (1/1) | Oil |
| Toluene | Oil (light yellow) |
| MeOH/water (1/1) | Form I |
| Acetone/water (1/1) | Gel-like |
| ACN/water (1/1) | Mostly amorphous |
| EtOH/MeCYH (1/1) | Oil |
| THF/MeCYH (1/1) | Oil |
| DCM/heptane (1/1) | Amorphous |
| EA/heptane (2/1) | Oil |
| 2-MeTHF/CYH (3/1) | Oil |
| IPAc/CYH (1/1) | Oil |
| Toluene/IPE (1/1) | Gel (brown) |
| IPE | New pattern |

### Example 7: Cooling

Quench cooling was performed in eight selected solvents. About 30 mg of starting material starting material was dissolved in selected solvent with sonication or stirring at 50° C. After hot filtration, the filtrate was cooled to RT directly. Any solid obtained was characterized by XRPD. The results are summarized in Table 29. Forms I, III and IV were obtained in quench cooling experiments.

TABLE 29
Results of Quench Cooling

| Solvent (v/v) | Result |
|---|---|
| MTBE | Form III |
| IPE | Amorphous |
| Toluene | Form I |
| IPE/toluene (1/1) | Form I |
| Acetone/water (3/10) | Form IV + I |
| IPA/water (1/4) | Form IV + I |
| EA/Heptane (20/13) | Form VI |

### Example 8: Anti-Solvent Precipitation

Anti-solvent precipitation was performed by adding anti-solvent dropwise to the prepared drug solution at RT. Appropriate amount of starting material was weighed into glass vials and then selected solvent was added to make nearly saturated solution. After filtration, anti-solvent was added into the filtrate gradually until solids precipitated out or 10V anti-solvent was added at RT. If precipitation occurred, solids were isolated by centrifugation and characterized accordingly. The results are summarized in Table 30. Forms II, III, IV, VI, and VII were obtained in anti-solvent precipitation experiments.

TABLE 30
Results of Anti-solvent Precipitation

| Solvent | Anti-solvent | Observation | V₁/V₂ | XRPD Result |
|---|---|---|---|---|
| MeOH | Water | Precipitation occurred | 1/2 | Form IV |
| EtOH | | immediately and became oily, | 1/2 | Form IV |
| Acetone | | Precipitation occurred after | 1/2 | Form VI |
| | | slurry for 1 h | 1/2 | |
| THF | | Precipitation occurred | 1/2 | Oil |
| ACN | | immediately and became oily | | Oil |
| EtOH | Heptane | Precipitation occurred | 1/2 | Form II |
| Acetone | | immediately and became | 1/2 | Oil |
| THF | | oily; | 1/1 | Similar to Form III |
| EA | | 013A9 precipitated out after | 1/1 | Form VI |
| DCM | | slurry for 2 h | 1/1 | Form VII + pks |
| MEK | MeCYH | Precipitation occurred after | 3/5 | Similar to Form VII |
| | | slurry for 1 min | | |
| 2-MeTHF | N/A | Dissolution-precipitation | 1/0 | Similar to Form III |
| | | occurred | | |
| IPA | N/A | Dissolution-precipitation | 1/0 | Form II |
| | | occurred | | |
| MIBK | CYH | Precipitation occurred after | 1/1 | Form VI |
| | | slurry for 1 min | | Form VII (15d) |
| IPAc | N/A | Dissolution-precipitation | 1/0 | Form VI |
| | | occurred | | |

V₁/V₂ is volume ratio of solvent to anti-solvent.

### Example 9: Solution Vapor Diffusion

Solution vapor diffusion was performed with heptane or MeCYH as anti-solvent. About 25 mg of starting material was dissolved in selected solvents to get a clear solution. The solutions were filtered into a clean vial and then placed in a 20-mL glass vial with 3 mL anti-solvent at RT, to allow vapor diffusion into the solution. Any solids obtained were characterized accordingly. The results are summarized in Table 31. Forms II and VII were obtained in solution vapor diffusion experiments.

TABLE 31
Results of Solution Vapor Diffusion

| Solvent | Anti-solvent | Result |
|---|---|---|
| IPA | Heptane | Similar to Form II |
| MEK | | Form VII |
| IPAc | | Oil |
| 2-MeTHF | | Form VII |
| MIBK | MeCYH | Oil |
| Anisole | | Oil |
| CPME | | Oil |

### Example 10: Salt Screening

Compound 1 has one very weak basic site with calculated pKₐ of 0.43. A salt screening was conducted with 6 pharmaceutically acceptable strong acids. About 25 mg of compound 1 was weighed into a 1.5-mL glass vial, then 1.1 eq. of selected acid was weighted into the above glass vial. Liquid strong acid was pre-diluted in corresponding solvent. After addition of 0.5 mL solvent, the mixture was stirred at RT for 24 h. 0.5 mL more solvent was added to dilute several viscous systems at 4 h (highlighted as * in the summary table). If no solid was obtained, anti-solvent of heptane was added into the filtrates gradually at RT to induce precipitation (highlighted as ** in the summary table). The suspensions were filtered and the solids were vacuum dried at 40° C. for 4 h.

Salt screening results are summarized in Table 32 and Table 33. ¹H-NMR results showed additional chemical shifts at around 8.5 ppm for salt samples with new XRPD patterns, suggesting potential chemical degradation. HPLC results (Table 33) confirmed significant purity decrease by>15%. The analysis results indicated that compound 1 was chemically unstable under strong acidic conditions.

TABLE 32
Summary of Salt Screening

| No. | Acid (eq.) | IPA | THF | MTBE |
|---|---|---|---|---|
| 0 | None | FB Form II ** | Oil ** | FB Form III * |
| 1 | HCl | Oil ** | Oil ** | New Pattern 3 |
| 2 | HBr | Oil ** | Oil ** | Oil * |
| 3 | Naphthalene-1,5- | Oil ** | Oil ** | New Pattern 3 |
| | disulfonic acid | | | |
| 4 | H₂SO₄ | Oil ** | Oil ** | New Pattern 3 |
| 5 | Ethane-1,2- | New Pattern 4 ** | Oil ** | FB Form III |
| | disulfonic acid | | | |
| 6 | p-Toluenesulfonic | Oil ** | Oil ** | Oil * |
| | acid | | | |

TABLE 33
Purity Results of Salt Samples

| Sample | Purity (area %) |
|---|---|
| Form I, starting material | 99.29 |
| New pattern 3, HCl salt | 76.10 |
| New pattern 3, naphthalene-1,5-disulfonate | 65.14 |
| New pattern 3, sulfate | 80.83 |
| New pattern 4, edisylate | 57.89 |

### Analysis Methods

### PLM

Light microscopy analysis was performed using an ECLIPSE LV100POL (Nikon, JPN) microscope. Each sample was placed on a glass slide with a drop of immersion oil and covered with a glass slip. The sample was observed using a 4-20×objective with polarized light.

### XRPD

XRPD diffractograms were collected with an X-ray diffractometer. The sample was prepared on a zero-background silicon wafer by gently pressing onto the flat surface. The parameters of XRPD diffraction are given in the table below.

Parameters for XRPD Testing

| | |
|---|---|
| Instrument | PANalytical, Empyrean |
| Radiation | Cu Kα (λ = 1.5418 Å) |
| Detector | PIXcel^{1D} |
| Scan angle | 3-40° (2θ) |
| Scan step | 0.013° (2θ) |
| Scan speed | 20.4 s/step |
| Tube voltage/current | 45 kV/40 mA |
| Divergence slit | ⅛° |
| Rotation | On |
| Sample holder | Zero-background sample pan |

### TGA

TGA analysis was performed using a TA Instrument. About 1-5 mg of a sample was loaded onto a pre-tared aluminum pan and heated with the parameters in the table below. The data was analyzed using TRIOS.

Parameters for TGA Testing

| Instrument | TA, Discovery TGA 55 | |
|---|---|---|
| Sample pan | Aluminum, open | |
| Temperature range | RT-300° | C. |
| Heating rate | 10° | C./min |

| | |
|---|---|
| Purge gas | N₂ |
| Flow rate | Balance chamber: 40 mL/min |
| | Sample chamber: 60 mL/min |

### DSC

DSC analysis was performed with a TA Instrument. About 1-3 mg of a sample was placed into an aluminum pan with pin-hole and heated with the parameters in the table below. The data was analyzed using TRIOS.

Parameters for DSC Analysis

| Instrument | TA, Discovery DSC 250 | |
|---|---|---|
| Sample pan | Aluminum, pin-holed | |
| Temperature range | 25-300° | C. |
| Heating rate | 10° | C./min |
| Purge gas | N₂ | |
| Flow rate | 50 | mL/min |

### DVS

Moisture sorption/desorption data were collected on a DVS instrument. Appropriate amount of sample was placed into a tared sample chamber and automatically weighed. The sample was analyzed with the setting parameters in the table below.

Parameters for DVS Analysis of Anhydrate

| Instrument | SMS, DVS Intrinsic | |
|---|---|---|
| dm/dt | 0.002%/min | |
| Sample size | 31 | mg |
| Measurement temperature | 25° | C. |
| Cycle | Full cycle | |
| Minimum dm/dt stability duration | 30 | min |
| Maximum dm/dt equilibrium time | 120 | min |
| Save data rate | 5 | s |
| Gas and Total flow rate | N₂, 200 sccm | |
| Post experiment total flow | 200 | sccm |

| | |
|---|---|
| RH step size | 10% RH |
| Method | Adsorption: 0, 10, 20, 30, 40, |
| | 50, 60, 70, 80, 90 |
| | Desorption: 80, 70, 60, 50, 40, |
| | 30, 20, 10, 0 |

### ¹H-NMR

¹H-NMR spectra were collected on a Bruker 400 MHz instrument. Unless specified, samples were prepared in DMSO-d6 or MeOH-d4 solvent and measured with the parameters in the table below. The data was analyzed using MestReNova.

Parameters for ¹H-NMR Analysis

| Instrument | Bruker | |
|---|---|---|
| Frequency | 400 | MHz |
| Scan times | 4 | |
| Temperature | 295 | K |
| Relaxation delay | 1 | s |

### HPLC

HPLC analysis was performed with an Agilent HPLC 1260 series instrument. HPLC methods for solubility and purity testing is presented in the tables below.

UPLC Method for Solubility Testing

| | |
|---|---|
| Instrument | Waters UPLC ACQUITY H-Class plus |
| Column | ACQUITY UPLC, BEH C18, 1.7 μm, 2.1 mm × 100 mm |
| Mobile phase | A: 0.05% TFA in H₂O; B: 0.05% TFA in ACN |
| Column Temp. | 40° C. |
| Gradient | 0.0/5%, 4.0/30%, 6/40%, 10/95%, 11/95%, 11.1/5%, |
| (T/B %) | 14/5% |
| Diluent | MeOH/H₂O (1/1, v/v) |
| Detector | PDA, 230 nm |
| Injection | 1 μL |
| volume | |
| Flow | 0.45 mL/min |

HPLC Method for Purity Testing

| | |
|---|---|
| Instrument | Agilent 1260 HPLC series |
| Column | XBridge shield RP 18 4.6 × 150 mm, 3.5 μm |
| Mobile phase | A: 0.05% TFA in H₂O; B: 0.05% TFA in ACN |
| Column Temp. | 40° C. |
| Gradient (T/B %) | 0.0/5%, 10.0/30%, 18/40%, 27/95%, 35/95%, 35.1/5% |
| Diluent | MeOH/H₂O (1/1, v/v) |
| Detector | DAD, 230 nm |
| Injection volume | 2 μL |
| Flow | 1 mL/min |

1. A crystalline form of (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1): 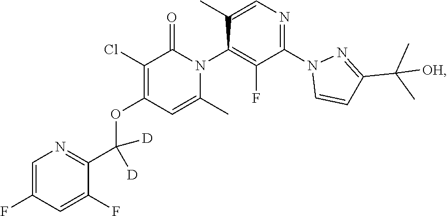
or a pharmaceutically acceptable salt or solvate thereof.

2. A crystalline form of freebase (M)-3-chloro-4-((3,5-difluoropyridin-2-yl)methoxy-d2)-3′-fluoro-2′-(3-(2-hydroxypropan-2-yl)-1H-pyrazol-1-yl)-5′,6-dimethyl-2H-[1,4′-bipyridin]-2-one (compound 1): 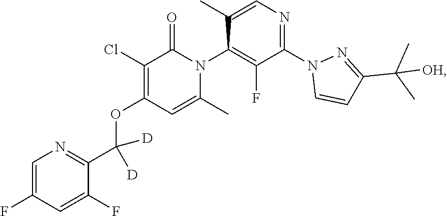
or a pharmaceutically acceptable solvate thereof.

3. (canceled)

4. The crystalline form of claim 1, wherein the crystalline form is selected from the group consisting of Form I of compound 1, Form II of compound 1, Form III of compound 1, Form IV of compound 1, Form VI of compound 1, Form VII of compound 1, Form VIII of compound 1, Form IX of compound 1, and Form X of compound 1, or any combinations thereof. cm 5-7. (canceled)

8. The crystalline form of claim 1, wherein the crystalline compound 1 is Form X characterized as having at least one of the following properties:
(a) an X-ray powder diffraction (XRPD) pattern substantially the same as shown in FIG. 6;
(b) an X-ray powder diffraction (XRPD) pattern with characteristic peaks at 9.17±0.2° 2θ, 16.29±0.2° 2θ, 21.67±0.2° 2θ, and 23.72±0.2° 2θ.
(c) a DSC thermogram with an endotherm having an onset temperature at about 157° C. and a peak temperature at about 158° C.; or
(d) combinations thereof.

9-10. (canceled)

11. The crystalline form of claim 8, wherein the X-ray powder diffraction pattern further comprises peaks at 13.44±0.2° 2θ, 14.95±0.2° 2θ, and 25.72±0.2° 2θ.

12-14. (canceled)

15. The crystalline form of claim 8, wherein the X-ray powder diffraction pattern further comprises at least one peak selected from 14.44±0.2° 2θ, 17.15±0.2° 2θ, and 18.74±0.2° 2θ.

16-20. (canceled)

21. The crystalline form of claim 1, wherein the crystalline compound 1 is Form I characterized as having at least one of the following properties:
(a) an X-ray powder diffraction (XRPD) pattern substantially the same as shown in FIG. 2;
(b) an X-ray powder diffraction (XRPD) pattern with characteristic peaks at 12.02±0.2° 2θ, 13.13±0.2° 2θ, 16.56±0.2° 2θ, 19.20±0.2° 2θ, 26.00±0.2° 2θ, and 28.00±0.2° 2θ;
(c) a DSC thermogram with an endotherm having an onset temperature at about 27° C. and a peak temperature at about 62° C.;
(d) a DSC thermogram with an endotherm having an onset temperature at about 98° C. and a peak temperature at about 104° C.;
(e) a thermogravimetric analysis (TGA) thermogram comprising a loss in mass of about 2.6% over a temperature range of about 25° C. to about 90° C.; or
(f) combinations thereof.

22-23. (canceled)

24. The crystalline form of claim 21, wherein the X-ray powder diffraction pattern further comprises at least one peak selected from 11.30±0.2° 2θ, 13.89±0.2° 2θ, 20.45±0.2° 2θ, and 26.39±0.2° 2θ.

25. (canceled)

26. The crystalline form of claim 21, wherein the X-ray powder diffraction pattern further comprises at least one peak selected from 9.21±0.2° 2θ, 9.57±0.2° 2θ, and 21.81±0.2° 2θ.

27-31. (canceled)

32. The crystalline form of claim 1, wherein the crystalline compound 1 is Form IV characterized as having at least one of the following properties:
(a) an X-ray powder diffraction (XRPD) pattern substantially the same as shown in FIG. 3;
(b) an X-ray powder diffraction (XRPD) pattern with characteristic peaks at 11.27±0.2° 2θ, 12.24±0.2° 2θ, 14.12±0.2° 2θ, 19.03±0.2° 2θ, 20.09±0.2° 2θ, 20.77±0.2° 2θ, 21.33±0.2° 2θ, and 26.37±0.2° 2θ.
(c) a DSC thermogram with an endotherm having an onset temperature at about 36° C. and a peak temperature at about 52° C.;
(d) a DSC thermogram with an endotherm having an onset temperature at about 103.5° C. and a peak temperature at about 109° C.;
(e) a thermogravimetric analysis (TGA) thermogram comprising a loss in mass of about 1.5% over a temperature range of about 33° C. to about 100° C.; or
(f) combinations thereof.

33-43. (canceled)

35. The crystalline form of claim 32, wherein the X-ray powder diffraction pattern further comprises at least one peak selected from 15.35±0.2° 2θ, 19.52±0.2° 2θ, 19.78±0.2° 2θ, 23.59±0.2° 2θ, 23.86±0.2° 2θ, and 27.88±0.2° 2θ.

36. (canceled)

37. The crystalline form of claim 32, wherein the X-ray powder diffraction pattern further comprises at least one peak selected from 15.51±0.2° 2θ, 15.79±0.2° 2θ, and 27.32±0.2° 2θ.

38-42. (canceled)

43. The crystalline form of claim 1, wherein the crystalline compound 1 is Form IV characterized as having at least one of the following properties:
(a) an X-ray powder diffraction (XRPD) pattern substantially the same as shown in FIG. 4;
(b) an X-ray powder diffraction (XRPD) pattern with characteristic peaks at 9.23±0.2° 2θ, 12.29±0.2° 2θ, 13.18±0.2° 2θ, 14.83±0.2° 2θ, 16.01±0.2° 2θ, 16.76±0.2° 2θ, 18.48±0.2° 2θ, and 26.84±0.2° 2θ.
(c) a DSC thermogram with an endotherm having an onset temperature at about 48° C. and a peak temperature at about 49° C.;
(d) a DSC thermogram with an endotherm having an onset temperature at about 154° C. and a peak temperature at about 155° C.;
(e) a thermogravimetric analysis (TGA) thermogram comprising a loss in mass of about 0.23% over a temperature range of about 30° C. to about 50° C.; or
(f) combinations thereof.

44-45. (canceled)

46. The crystalline form of claim 43, wherein the X-ray powder diffraction pattern further comprises at least one peak selected from 10.43±0.2° 2θ, 12.94±0.2° 2θ, 17.99±0.2° 2θ, 19.57±0.2° 2θ, 21.80±0.2° 2θ, and 27.16±0.2° 2θ.

47. (canceled)

48. The crystalline form of claim 43, wherein the X-ray powder diffraction pattern further comprises at least one peak selected from 13.74±0.2° 2θ, 19.14±0.2° 2θ, and 19.96±0.2° 2θ.

49-53. (canceled)

54. The crystalline form of claim 1, wherein the crystalline compound 1 is Form IX characterized as having at least one of the following properties:
(a) an X-ray powder diffraction (XRPD) pattern substantially the same as shown in FIG. 5;
(b) an X-ray powder diffraction (XRPD) pattern with characteristic peaks at 7.59±0.2° 2θ, 13.18±0.2° 2θ, 13.95±0.2° 2θ, 15.48±0.2° 2θ, 18.78±0.2° 2θ, 20.87±0.2° 2θ, 21.62±0.2° 2θ, 23.37±0.2° 2θ, and 28.07±0.2° 2θ.
(c) a DSC thermogram with an endotherm having an onset temperature at about 29° C. and a peak temperature at about 55.5° C.;
(d) a DSC thermogram with an endotherm having an onset temperature at about 113.5° C. and a peak temperature at about 118° C.;
(e) a thermogravimetric analysis (TGA) thermogram comprising a loss in mass of about 1.39% over a temperature range of about 27° C. to about 80° C.; or
(f) combinations thereof.

55-56. (canceled)

57. The crystalline form of claim 54, wherein the X-ray powder diffraction pattern further comprises at least one peak selected from 17.65±0.2° 2θ, 19.19±0.2° 2θ, 20.14±0.2° 2θ, 23.54±0.2° 2θ, 26.65±0.2° 2θ, and 30.1±0.2° 2θ.

58. (canceled)

59. The crystalline form of claim 54, wherein the X-ray powder diffraction pattern further comprises at least one peak selected from 11.09±0.2° 2θ, 27.49±0.2° 2θ, and 30.99±0.2° 2θ.

60-64. (canceled)

65. A pharmaceutical composition comprising a therapeutically effective amount of a crystalline form of claim 1 and a pharmaceutically acceptable excipient.

66. A method for treating a condition comprising administering to a subject in need thereof a therapeutically effective amount of a crystalline form of claim 1, wherein the condition is selected from the group consisting of an autoimmune disorder, a chronic inflammatory disorder, an acute inflammatory disorder, an auto-inflammatory disorder, a fibrotic disorder, a metabolic disorder, a neoplastic disorder, and a cardiovascular or a cerebrovascular disorder.

67. A method of treating a p38 MAP kinase-mediated disease in a subject in need thereof comprising administering to the subject a therapeutically effective amount of a crystalline form of claim 1.

68. A method of treating a MK2-mediated disease in a subject in need thereof comprising administering to the subject a therapeutically effective amount of a crystalline form of claim 1.
